(19) United States
(12) Patent Application Publication
(10) Pub. No.: US 2025/0248752 A1
(43) Pub. Date: Aug. 7, 2025

(54) INTEGRATED MODULATION OF CELLULAR METASTASIS

(71) Applicant: EMERALD GATE CHARITABLE TRUST, Jackson, WY (US)

(72) Inventors: Peiying Yang, Sugar Land, TX (US); Lorenzo Cohen, Houston, TX (US); Jason Yotopoulos, San Juan, PR (US)

(73) Assignee: EMERALD GATE CHARITABLE TRUST, Jackson, WY (US)

(21) Appl. No.: 19/043,157

(22) Filed: Jan. 31, 2025

Related U.S. Application Data
(60) Provisional application No. 63/548,571, filed on Feb. 1, 2024.

(51) Int. Cl. A61B 18/00

(52) U.S. Cl. CPC A61B 18/00; A61B 2018/00839

(57) ABSTRACT

Provided herein are systems and methods for decreasing metastatic potential of cells and/or adjusting biological functioning of an entity by effecting a change in at least one biophysical (e.g. bioelectrical) feature, at least one structural feature, and at least one physiological feature of cells. The systems and methods can include administration of biofield therapy, and in some embodiments alter expression of genes associated with the changes in bioelectrical, structural, and/or physiological features.

### BACKGROUND OF THE INVENTION

This invention relates to integrated effects, assessments, and mechanisms, and particularly bioelectric-, energy- and/or information-based effects and mechanisms, for modulating structure and function of cells to address cancerous states and/or functionality, and applications thereof.

Cancer remains a major cause of death, second only to heart disease overall and now the number one cause of death for those under 85 years old, and accounting for almost one in every six deaths in the US. Despite decades of research, five-year survival rates for lung, liver, esophageal, and pancreatic cancer remain below 25%. Pancreatic cancer is currently the third leading cause of cancer-related death in the US and patient prospects are particularly dismal, with less than 6 months average survival duration from diagnosis.

Cancer cells generally have increased rates of growth relative to normal cells and may have an increased potential for metastasis, which contributes to the spread of cancer within a patient. Cancer can be understood as a loss of organization capacity of the environment over behavior of isolated cells (Wang I C, et al., Molecular and Cellular Biology. 2005; 25(24):10875-10894; Myatt S S, et al., Nature Reviews Cancer. 2007; 7(11):847-859; Anders L, et al., Cancer Cell. 2011; 20(5):620-634) and/or a dysregulation of cellular pathways, cell communication, and cell state. For example, some cancer cell types have a depolarized cell voltage potential. Some cancer cell types, including pancreatic cancer, may have elevated expression of certain proteins that drive the metastatic processes.

Most current approved methods for the treatment of cancer are toxic to the patient and generally do not restore non-cancerous states and/or functionality to cancer cells or preserve homeostasis of potentially cancerous cells. Current approved methods for the treatment of cancer target only one or two abnormal proteins or pathways and are limited in biological scope and may lead to treatment resistance.

### SUMMARY OF THE INVENTION

Provided herein are systems and methods for adjusting biological functioning of cells by effecting, directly or indirectly, each of a biophysical change, a structural change, and a physiological change in some or all of the cells. The cells can be in vitro or in vivo. The systems and methods can be used to treat cancerous cellular activity (cancer) in organisms and/or to evaluate treatments and/or therapists.

In one embodiment, the systems and methods induce a biophysical change, e.g., a change in a bioelectrical feature, of cells (e.g., a first portion of cells). A biophysical change, e.g., a change in a bioelectrical feature, can effect a structural and/or a physiological change in the same cells (the first portion of cells) or in other cells (a second portion of cells). In one embodiment, the first portion of cells includes immune cells. In one embodiment, the second portion of cells includes cancerous or tumor cells. Generally, each of the biophysical change, the structural change, and the physiological change can effect changes in one or more aspects of one or both of the other categories, e.g., in each other. Each of the biophysical change, the structural change, and the physiological change can be correlated with one or both of the other. In some embodiments, the systems and methods further comprise altering of expression of genes associated with the change in the bioelectrical feature, the change in the structural feature, and/or the change in the physiological feature. The altering of expression of genes may comprise an epigenetic effect, a change in transcriptomic and/or proteomic signature for the bioelectrical feature, the structural feature, and/or the physiological feature

In certain embodiments, the systems and methods reduce metastatic potential of cells (including without limitation reducing an incidence and/or likelihood of metastasis, wherein metastasis includes and/or can be characterized by, without limitation, invasion and/or migration of cells) and/or reducing growth or size of tumors. In certain embodiments, one, two or each of the biophysical change, the structural change, and the physiological change is/are correlated with at least one, two, three, four, five, or all of: limiting progression through the cell cycle or cell cycle “arrest” (e.g., cells remain longer in the G0/G1 and/or G2/M phases of the cell cycle) and/or expression of cell cycle regulation proteins; decreased cell-free DNA or intravasation; decreased growth and/or metabolism of the cells; deceased migration of the cells; suppressed stemness; and decreased invasion of the cells.

Effecting a biophysical change or a change in a biophysical aspect can include at least one, two, three, or all of: altering intracellular ion levels of the cells (e.g., altering, e.g., reducing, intracellular calcium ion levels of the cells); altering cell membrane potential of the cells (e.g., reducing cell membrane potential of the cells and/or hyperpolarizing the cells); altering membrane potential of the mitochondria (e.g., reducing mitochondrial membrane potential); and altering of expression of ion channel genes (including but not limited to genes for proteins of or associated with a voltage-gated ion channel, a sodium channel, a potassium channel, and/or a calcium-regulated channel, e.g., Nav1.5,Kv1.3 and/or KCa3.1).

Effecting a structural change or a change in a structural aspect can include at least one, two, three, four, or all of: changing a structural feature of the mitochondria (e.g., size, swelling, degree of organization of cristae); changing cytoskeleton organization of the cells (e.g., decreasing cytoskeleton structure, regulating activity of actin, including F-actin and/or beta actin, and/or tubulin); altering membrane ion channels of the cells (e.g., voltage-gated ion (e.g., calcium and/or potassium) channels in the cellular membrane, e.g., altering membrane ion proteins such as CFTR, GABARP, and/or Ryanodine receptor 3, e.g, altering (e.g., reducing) a cholinergic receptor anion channel protein, e.g., CFTR, altering (e.g., reducing) a GABA receptor protein, e.g., GABRP, and/or altering (e.g., decreasing) a calcium-releasing protein, e.g., Ryanodine receptor 3) and/or altering the expression of genes encoding one or more membrane ion proteins (e.g., CFTR, GABARP and/or RYR3); altering of expression of cytoskeletal genes (e.g., activating cytoskeleton-related genes, e.g., genes for actin, pectin, spectrin, TRIOBP, kinases, and/or PDZ); and altering of expression of genes involved in communication with the cytoskeleton (e.g., altering communication factor to cytoskeleton (PDZ).

Effecting a physiological change or a change in a physiological aspect can include one, two, three, four, five, six, seven, or all of: altering one or more EMT marker proteins in the cells (e.g., increasing epithelial marker E-cadherin and/or reducing mesenchymal marker N-cadherin and/or Twist 1); regulating (e.g., decreasing levels or reducing activity of) FOXM1 in the cells; altering energy metabolism in mitochondria of the cells (e.g., altering mitochondrial function, expression of protein kinase and/or ATP-binding genes, and/or glucose and glutamine metabolism); altering of expression of one or more EMT genes (e.g., decreasing expression of N-cadherin, decreasing expression of CD44, and/or increasing expression of E-cadherin); altering of expression of genes for one or more FOXM1-associated proteins (e.g., decreasing expression of pAKT, ALKBH5, cyclin B1, and/or CDK1; and/or increasing expression of P21); altering of expression of genes for one ore more serine/threonine kinases; altering of expression of genes for cell junction; and altering of expression of genes for extracellular matrix.

In some embodiments, the systems and methods further include effecting an additional change in a biophysical, structural, and/or physiological aspect. Effecting an additional change can include one, two, three, four, five or all of: regulating hypoxia and/or apoxia of the cells (e.g., decreasing protein expression of one or more hypoxia markers, HIF-1, and/or PDK1); altering of expression of genes associated with Ras pathway, extracellular matrix, serine/threonine kinase, and/or cell junction; altering cell signaling (e.g., alteration of microtubules and/or cytoskeletal markers); regulating PI3K/mTOR pathway in the cells (e.g., reducing levels of pAkt and/or the ratio of pAkt to total Akt); and altering levels of immune cells (e.g., B cells and/or T cells, e.g., reducing B cells and/or increasing T cells); when cells are component(s) of an organism experiencing cancer, decreasing nodules (e.g., reducing the number or visibility of nodules) and/or mets, e.g., decreasing liver nodules and/or mets to the liver; and, when cells are components of an organism having a tumor, changing the tumor microenvironment (TME) (e.g., reducing the percentage of B lymphocytes, increasing the percentage of cytotoxic T-cells, and/or reducing M2 tumor associated macrophages).

In some embodiments, the systems and methods include administering a biofield therapy, e.g., Bengston Energy Healing Method. The biofield therapy may induce or effect a biophysical change (e.g., a change in a bioelectrical feature), a structural change, and/or a physiological change, either directly or indirectly. In some embodiments, administering the biofield therapy is correlated with at least one, at least two, or all of: the biophysical change, the structural change, and the physiological change. In some embodiments that include administering a biofield therapy, e.g., to an organism, the biophysical change, the structural change, and/or the physiological change are correlated with decreased migration and/or invasion (e.g., of the cells and/or decreased tumor size or burden. The decreased migration and/or invasion of the cells can be achieved without toxic effects to the cells or a host of the cells. In some embodiments, the biofield therapy or a portion thereof is administered for about 15 minutes or about 30 minutes in a day. In some embodiments, the biofield therapy or a portion thereof is administered at least two or at least three days in a week. The biofield therapy can be administered locally or at a distance, in whole or in part. In some embodiments, the biofield therapy is administered by more than one therapist. A portion (including part or all) of the biofield therapy can be administered by a group of therapists.

The administration (e.g., length of time of administration and/or frequency of administration) and/or administrator of the biofield therapy can be determined according to an indicator of efficacy of the person performing the biofield therapy and/or the manner of performance of the biofield therapy. An indicator of efficacy of a biofield therapist can be based on a biophysical, structural, or physiological change, including the combinations and examples of effects as identified herein (e.g., above and in the claims as filed), in one or more entities treated. An indicator of efficacy of a biofield therapist can be based on or reflect measurements of the electrical activity of the brain of the therapist (e.g., an EEG). An indicator of efficacy of a biofield therapist can be based on or reflect the degree of correlation of changes in electrical activity of the brain of the therapist (e.g., an EEG) with any of the effects and/or integrations of effects described herein, including the combinations and examples of effects as identified herein (e.g., above and in the claims as filed). In one embodiment, calcium signaling and/or beta-actin of cells being treated are correlated with changes in an EEG of the therapist.

The systems and methods can include administering a biofield therapy and a chemical agent and/or radiotherapy. In such embodiments, administering a biofield therapy can ameliorate deleterious effects of the administration of the chemical agent and/or the radiotherapy. In such embodiments, administering a biofield therapy can increase the efficacy of treatment with the chemical agent and/or the radiotherapy.

Also provided herein are systems and methods for improving cell function, e.g., decreasing growth and/or metastatic potential of cells and/or cancer cells, by at least one, at least two, at least three, at least four, at least five, at least six, at least seven, at least eight, or all of: altering membrane potential (e.g., membrane potential is reduced and/or cells become hyperpolarized); changing cytoskeleton organization (e.g., decreasing cytoskeleton structure, regulating activity of actin and/or tubulin); regulating energy metabolism (e.g., altering mitochondrial function, expression of protein kinase and/or ATP-binding genes, and/or glucose and glutamine metabolism); altering morphology of mitochondria (e.g., size, swelling, degree of organization of cristae); limiting progression through the cell cycle (e.g., cells remain longer in the G0/G1 and/or G2/M phases of the cell cycle); regulating hypoxia and/or apoxia (e.g., decreasing protein expression of one or more hypoxia markers, HIF-1, and/or PDK1); regulating (e.g., decreasing levels or reducing activity of) FOXM1 and/or PI3K/mTOR pathways; regulating epigenetics processes (e.g., causing a shift in expression of genes in the cells including genes associated with Ras pathway, extracellular matrix, cell junction and cytoskeleton); and changing EMT marker expression of the cancer cells (e.g., increasing epithelial marker E-cadherin and/or reducing mesenchymal marker N-cadherin and/or Twist 1). The methods can include, and the effects may occur as the result of, administration of biofield therapy.

In certain embodiments, decreasing metastatic potential includes one, two, three, four, or all of: decreasing growth and/or metabolism of cells; decreasing size of a tumor comprising cells; reducing the likelihood of occurrence of metastasis; reducing migration or other metastatic behaviors of cells or their progeny; and reducing penetration or other metastatic capabilities of cells or their progeny.

In certain embodiments, the systems and methods comprise two or all of: altering bioelectricity (e.g., altering membrane potential of the cells or mitochondria in the cells, e.g., reducing membrane potential, and/or hyperpolarizing the cells); changing cytoskeleton organization in the cells (e.g., decreasing cytoskeleton structure, regulating activity of actin, including beta-actin and/or F-actin, and/or tubulin); and regulating (e.g., decreasing levels or reducing activity of) FOXM1 in the cells. For example, the systems and methods comprise regulating FOXM1 in the cells and changing cytoskeleton organization in the cells. In one embodiment, regulation of FOXM1 is achieved without toxic effects to the cells and/or a host of the cells. In one embodiment, the method includes administration of biofield therapy and FOXM1 in the cells is reduced. In one embodiment, a reduction of FOXM1 in the cells is correlated with a reduction in migration and/or invasiveness of the cells.

The cells of the systems and methods described herein can comprise cells in or from an organoid, e.g., a patient-derived and/or a pancreatic cancer organoid. The cells can comprise pancreatic ductal adenocarcinoma (PDAC) cells. The cells can include cells from a cell line, e.g., one or more of PANC-1, Panc02, MiaPaca, Capan-2, KPCY, COLO357, and L3.7 cell lines. In certain embodiments, the cells are human cells. In certain embodiments, the cells are murine cells. In certain embodiments, the cells are component(s) (exist as part of) an organism, e.g., a human or mouse.

The systems and methods provided herein also include methods for determining efficacy of a therapy, including efficacy of a therapy and/or an administrator of or device for administration of therapy, e.g., a therapy administration (e.g. a dosage or treatment regimen), a biofield therapy technique, a therapist, or a device for administering biofield therapy or its equivalents, for example, according to an indicator of efficacy. The indicator of efficacy can comprise and/or be based upon the effects and integrations of effects described herein, including without limitation the combinations and examples of effects as identified herein (e.g., above and in the claims as filed). For example, the indicator of efficacy can comprise and/or be based upon a measure indicating decreased or downregulation of FOXM1, for example, relative to a standard or a prior measurement. The indicator of efficacy can comprise and/or be based upon a measure indicating at least one, two, three, four, five, six, seven, or all of altered membrane voltage potential, changed cytoskeleton organization, regulation of energy metabolism, limited progression through the cell cycle, regulated hypoxia and/or apoxia, regulated PI3K/mTOR pathway, regulating epigenetics processes, and changed EMT marker expression of the cancer cells. The indicator of efficacy can comprise and/or be based upon measures of at least one, two, three, four, five, six, seven, eight, nine, ten, eleven, twelve or more of: membrane ion channels, cell membrane voltage potential, membrane potential of the mitochondria, expression of voltage-gated ion channel genes, structure of the mitochondria, cytoskeleton organization of the cells, intravasation of the cells, expression of cytoskeletal genes, EMT markers, FOXM1, energy metabolism in mitochondria of the cells, expression of one or more EMT genes, and expression of one or more FOXM1-associated genes, including as exemplified above and herein.

The systems and methodologies described herein can be used in the treatment of and/or to devise or assess treatments (e.g., evaluate and/or compare possible effectiveness of treatments) of cells in tumors and/or cells that may metastasize, including but not limited to pancreatic cancer cells. The systems and methodologies can be used in the treatment of and/or to devise or assess treatments (e.g., evaluate and/or compare possible effectiveness of treatments) of, for example, cancers and/or cancer cells or cell-lines. In certain embodiments, the cancers or cell-lines have high FOXM1 expression, e.g., BRCA, CESC, LUAD, STAD and THCA tumor tissues. The use of the systems and methodologies in the treatment of and/or to devise or assess treatments can comprise and/or be based upon any of the effects and/or integrations of effects described herein, including without limitation the combinations and examples of effects as identified herein (e.g., above and in the claims as filed).

### BRIEF DESCRIPTION OF DRAWINGS

FIG. 1. Factors that may be involved in or affected by mechanisms for cellular homeostasis and/or maintenance or recovery of non-cancerous cell states, including factors that may be characterized as structural, physiological and/or biophysical.

FIG. 2. An exemplary comparison of the influence of each of three forms of treatment (pharmacological, radiation, and biofield) on the structural features (e.g., cytoskeleton), physiological features (e.g., protein pathway and epigenetic regulation) and biophysical features (e.g., membrane potential) of cells. Biofield therapy influences each of these three aspects of cells in an integrated manner that rebalances cell structure and function, whereas pharmacological and radiation therapy may target one or more aspects to the detriment of others.

FIG. 3. A summary of mechanisms of action of treatment with BT on cellular structure, physiology and biophysics, and consequent effects on certain properties and indicators of cancer, including electrical and molecular mechanisms of action and interrelations among them.

FIG. 4. (A) Effect of biofield therapy (BT) on the growth of pancreatic ductal adenocarcinoma cells, including human PANC-1 (a and b) and COLO357 cells (c), mouse Panc02 cells, and immortalized human pancreatic ductal epithelial cells (HPDE-1/E6E7; d) treated by biofield Therapist 1 and PANC-1 (f), COLO357 (g), L3.7 (h), and HPDE cells (i) treated by biofield Therapist 2, compared with sham control (SC) and incubator control (IC). (B) Effect of BT on the growth of human patient-derived pancreatic cancer organoids (PDOs, AM67): (a) The average number of PDOs AM67 11 days after BT (15 minutes and 30 minutes) or SC; (b) The size of the PDOs AM67 after BT treatment (15 minutes) or SC. Data are presented as mean±SD (*p<0.05; **p<0.01; ***p<0.001; ****p<0.0001; n>4).

FIG. 5. Effect of biofield therapy (BT), at 1 hour after 15 minutes of treatment, on the growth of PANC-1 cells (A-C) in five different sets of experiments and Panc02 cells (D-F) in three different sets of experiments, compared with sham control (SC) and incubator control (IC). Data are presented as mean±SD (*p<0.05; **p<0.01; ***p<0.001; ****p<0.0001; n>4).

FIG. 6. Effect of biofield therapy (BT), compared with sham control (SC) and incubator control (IC), on the growth of MiaPaCa-2 (A) and KPCY cells (B). Data are presented as mean±SD (*p<0.05; **p<0.01; ***p<0.001; n>4).

FIG. 7. Effect of biofield therapy (BT), compared with sham control (SC) and incubator control (IC), on the growth of PANC-1 cells treated by Therapist 3. Data are presented as mean±SD (*p<0.05; ***p<0.001; n>4).

FIG. 8. Transmission electron micrographs showing morphologic changes in PANC-1 cells in the incubator control group (A and D), sham control group (B and E), and biofield therapy group (C and F) after 15 minutes of exposure to treatment (samples were collected immediately after treatment). Note the normal appearance mitochondria (red arrows) in the incubator control and sham control groups (D and E) and swollen mitochondria in the biofield therapy group (yellow arrow, F). Images A-C were taken at 5000×; images D-F were taken at 25,000×.

FIG. 9. Cell cycle analysis of PANC-1 cells. (A-C) PANC-1 cells were plated in 6-well plates overnight and then exposed to biofield therapy (BT), sham control (SC), or incubator control (IC) for 15 minutes. Cells were collected immediately after the treatment. Cell cycle analysis was carried out with propidium iodide staining and flow cytometry. (A) Histogram of cell cycle populations of PANC-1 cells in the IC, SC, and BT groups after 15-minute exposures. (B and C) Quantitative analysis of PANC-1 cells in different cell cycle phases after 15-minute BT, SC, or IC in two separate experiments. (D) PANC-1 cells were plated in 6-well plates for 48 hours prior to BT (15 minutes). (E and F) L3.7 cells and COLO357 cells were plated overnight and then exposed to BT for 30 minutes. Data are presented as mean±SD (*p<0.05; **p<0.01; ***p<0.001; ****p<0.0001; n=3).

FIG. 10. (A and B) Effect of biofield therapy (BT), sham control (SC), or incubator control (IC) on the cell cycle of PANC-1 cells in another two separate studies. (C and D) Cell cycle of PANC-1 cells after 15-minute exposure to BT provided by Therapist 2 (C) and Therapist 3 (D). Data are presented as mean±SD (*p<0.05; **p<0.01; ***p<0.001; ***p<0.0001; n>4).

FIG. 11. Cell membrane voltage potential of pancreatic ductal adenocarcinoma cells. (A) Representative fluorescence imaging of DiBAC4 and Hoechst in PANC-1 cells under IC, SC, and BT (15 minutes) conditions. (B) Quantitative analysis of cell voltage in PANC-1 cells after 15-minute BT. (C) Quantitative analysis of cell voltage after 15-minute BT for two consecutive days. (D) Cell voltage of MiaPaCa-2 cells measured at 1 hour after BT. (E) Cell voltage of KPCY cells measured at 1 hour after BT. Data are presented as mean±SD (*p<0.05; **p<0.01; ***p<0.001; n>4).

FIG. 12. Cell membrane voltage potential of (A) MiaPaCa-2 and (B) KPCY cells measured immediately after biofield therapy (BT), sham control (SC), or incubator control (IC). Data are presented as mean±SD (**p<0.01; ***p<0.001; n>6).

FIG. 13. Biofield therapy (BT) markedly inhibited invasiveness and migration of PDAC cells compared with sham control (SC) and incubator control (IC). (A) Invasiveness of PDAC cells: PANC-1 (a), L3.7 (b), COLO357 (c), KPCY (d), and migration of PANC-1 (e) and L3.7 (f) cells. (B) Migration of the MiaPaCa-2 cells via scratch assay, with data shown for 48 hour study. (C) Invasiveness of PANC-1 (a), COLO357 (b), and L3.7 (c) cells and migration of PANC-1 (d) and L3.7 (e) cells treated by Biofield Therapist 2. Data are presented as mean±SD (*p<0.05; ***p<0.001; ****p<0.0001).

FIG. 14. Biofield therapy (BT) significantly inhibited the invasiveness of PANC-1 cells (A-D) and L3.7 cells (E) after they were treated by Therapist 1 for 15 or 30 minutes, compared with sham control (SC). (F) Invasiveness of PANC-1 cells incubated in the SC or BT incubator. Data are presented as mean±SD (**p<0.01; ***p<0.001; ****p<0.0001).

FIG. 15. Expression of proteins that regulate the cell cycle and cell signaling in PANC-1 cells exposed to biofield therapy (BT) for 15 minutes, compared with sham control (SC) and incubator control (IC). (A) Heatmap of cell cycle-regulating proteins and cell signaling proteins in PANC-1 cells, analyzed by reverse phase protein array (RPPA). The proteins included in this heatmap were statistically significant among different treatment groups according to Wilcoxon rank-sum analysis, with p values lower than the Benjamini-Hochberg thresholds for false discovery rate (<0.05). (B) Expression of cell cycle-regulating proteins and epigenetic modification proteins, such as CDK1, cyclin B1, FOXM1, and ALKBH5, in PANC-1 cells exposed to BT compared with SC, as examined by RPPA (n=7). (C and D). Western blot analysis of FOXM 1 (C) and p21 (D) in PANC-1 cells exposed to BT for 15 minutes. Data are presented as mean±SD (*p<0.05; ***p<0.001; n=7).

FIG. 16. Expression of proteins that regulate the cell cycle and cell signaling in PANC-1 cells exposed to biofield therapy (BT) for 30 minutes, compared with sham control (SC). (A) Heatmap of cell cycle-regulating proteins and cell signaling proteins in PANC-1 cells, as examined by reverse phase protein array (RPPA). The proteins included in this heatmap were statistically significant among different treatment groups according to Wilcoxon rank-sum analysis, with p values lower than the Benjamini-Hochberg thresholds for false discovery rate (<0.05). (B) Expression of cell cycle-regulating proteins and epigenetic modification proteins such as cyclin B1, FOXM1, and TRIM24 in PANC-1 cells exposed to BT, compared with SC, as examined by RPPA (n=7).

FIG. 17. Expression of FOXM1 (A) and p21 (B) proteins in L3.7 cells exposed to biofield therapy (BT), compared with sham control (SC) and incubator control (IC), examined by Jess. Data are presented as mean±SD (*p<0.05; ***p<0.001).

FIG. 18. Effect of biofield therapy (BT), compared with sham control (SC) and incubator control (IC), on epithelial-mesenchymal transition in PANC-1 cells. Western blot analysis of E-cadherin (A), N-cadherin (B), and CD44 (C) was conducted in PANC-1 cells immediately after 15-minute BT. Data are presented as mean±SD (*p<0.05; **p<0.01; ****p<0.0001).

FIG. 19. The percentage of FOXM1 inhibition from BT by BT's percentage inhibition of the invasiveness of PDAC cells, and the respective correlation.

FIG. 20. Modification of FOXM1 in PANC-1 cells altered the cell cycle of the PANC-1 cells and biofield therapy (BT) elicited anti-invasiveness compared with sham control (SC) in PDAC cells. (A) Western blot analysis of FOXM1 in wildtype PANC-1 cells, FOXM1 stable-knockdown PANC-1 cells, and FOXM1-overexpressing (OE) PANC-1 cells. (B) Cell cycle occurrence of FOXM1 knockout PANC-1 cells for SC, IC and BT compared with parental (“WT”) PANC-1 cells. (C) Invasiveness of parental PANC-1 cells and FOXM1-knockdown (KD) PANC-1 cells exposed to BT. (D) Invasiveness of wild-type (WT) L3.7 cells and FOXM1-knockout L3.7 cells exposed to BT. (E) Invasiveness of WT PANC-1 cells and FOXM1 knockout PANC-1 cells exposed to BT. (F) Invasiveness of WT COLO357 cells and FOXM1-overexpressing COLO357 cells exposed to BT. (G) Invasiveness of COLO357cells and FOXM1 conditional-overexpressing COLO357 cells exposed to BT. (Data are presented as mean±SD (*p<0.05; **p<0.01; ***p<0.001; ****p<0.0001).

FIG. 21. Biofield therapy (BT) inhibited the growth of primary pancreatic tumors and significantly reduced liver metastasis (i.e., metastasis of cells to the liver) compared with sham control (SC) and colony control (CC) in a human PANC-1 cell mouse orthotopic model. (A) Terminal tumor weight of PANC-1 tumors after exposure to BT, control, or gemcitabine for 3 weeks and observation for an additional 7 weeks. (B) Percentage of the PANC-1 tumor-bearing mice with liver metastasis at the termination of the study. (C) Average number of liver nodules in PANC-1 tumor-bearing mice with liver metastasis. (D) Representative pictures of liver tissues collected from CC, SC, and BT mice. Red arrows indicate liver nodules. (E) Representative hematoxylin and eosin-stained images of mouse liver tissues in the SC and BT mice bearing PANC-1 orthotopic tumors. (F) Percentage of the PANC-1 tumor-bearing mice with liver metastasis at the termination of the repeated study. (G) Average liver tumor burden in PANC-1 tumor-bearing mice with liver metastasis in the repeated study. Data are presented mean+SE (*p<0.05; n>12 mice per group).

FIG. 22. FOXM1 protein expression in L3.7 wild-type and L3.7 CRISPR-Cas9 FOXM1 knockdown clones, examined by Jess.

FIG. 23. Effect of FOXM1 overexpression on biofield therapy (BT) elicited anti-invasiveness in PANC-1 cells. (A) Invasiveness of control PANC-1 and FOXM1 stable overexpressing PANC-1 cells exposed to sham control (SC) or BT. (B) Invasiveness of control PANC-1 and Tet-inducible FOXM1 overexpressing PANC-1 cells exposed to SC or BT. Datae are presented as mean±SD. **p<0.01; ***p<0.001; ****p<0.0001).

FIG. 24. The Effect of biofield therapy (BT) on marker of epithelial-mesenchymal transition in PANC-1 cells and intravasation of PANC-1 tumor bearing mice. (A) Gene expression of Twist1 in PANC-1 cells after being treated with BT for 15 min. (B) Cell free DNA in plasma of PANC-1 tumor bearing mice from SC and BT group. Data are presented as mean±SD. *p<0.05; **p<0.01; ***p<0.001; ****p<0.0001.

FIGS. 25A and 25B. Transmission electron micrographs showing morphologic changes in PANC-1 cells in the IC group (a and d), sham control group (b and e), and biofield therapy group (c and f) after 15 (A) and 30 minutes (B) of exposure to treatment (samples were collected immediately after treatment). Note the normal appearance mitochondria (red arrows) in the incubator control and sham control groups (d and e) and swollen mitochondria in the biofield therapy group (yellow arrow, f). Images a-c were taken at 5000×; images d-f were taken at 25,000×.

FIG. 26. The role of FOXM1 in BT elicited anti-invasiveness of PDAC cells. Western blot analysis of FOXM1 in PANC-1 cells (WT), FOXM1 stable-knockdown PANC-1 cells (KD), or FOXM1-overexpressing (OE) PANC-1 cells. Data are presented as mean±SD (**p<0.01; ***p<0.001; ****p<0.0001).

FIG. 27. The effect of BT on voltage gated ion potassium channels in PANC-1 and cell voltages of Panc-1 cells and FOXM1 KO PANC-1 cells. (A). Expression of voltage gated potassium channels (VGKC) and voltage gated sodium channel genes in PANC-1 cells treated with BT for 15 min and collected immediately after the treatment. Expression of SCN5A (Nav1.5, B), KCNA3 (Kv1.3, C), and KCNN4 (KCa3.1, D) genes in 15- and 30-minutes treated PANC-1 cells measured by realtime qPCR. (E) Cell membrane voltage potential was measured by DiABC4 staining in the PANC-1 and FOXM1 KO PANC-1 cells. Data are presented as mean±SD. *p<0.05, **p<0.01, ***p<0.001, ****p<0.0001.

FIG. 28. Biofield therapy (BT) significantly reduced liver metastasis compared with sham control (SC) and colony control (CC) in a human PANC-1 cell mouse orthotopic model and KPCY 6419 syngeneic mouse model. (A) Terminal tumor weight of PANC-1 tumors after exposure to BT, control, or gemcitabine for 3 weeks and observation for an additional 7 weeks. (B) Percentage of the PANC-1 tumor-bearing mice with liver metastasis at the termination of the study. (C) Average liver tumor burden in PANC-1 tumor-bearing mice with liver metastasis. (D) Representative pictures of liver tissues collected from CC, SC, and BT mice. Red arrows indicate liver nodules. (E) Percentage of visible liver metastasis in KPCY 6419 mouse model after exposure to CC, SC, BT and gemcitabine. (F) Liver metastasis in KPCY 6419 mouse model quantified by imaging the YFP+ cells in the liver tissues. (G) Representative images of liver tissues collected from WT, SC, and BT mice. Blue arrows indicate liver nodules. Data are presented mean±SE (*p<0.05; n>12 mice per group).

FIG. 29. Immune profile of KPCY tumor after being treated with BT. A) Percentage of CD19+ B cells among total of parental cells; B) Percentage of CD4+ T cells among of total of parental cells; C) Percentage of CD8+ T-cells over CD45+ T cells; and E) Percentage of CD4+/FOXP3+ T-reg cells among the CD45+ T cells. **p<0.01 versus SC control.

FIG. 30. The effect of faraday cage on the cell cycle and cell invasion of the PDAC cells treated with BT. A) Experimental setting of the faraday cage study with cells either placed on the bench (right) or inside the faraday cage box (left). B) Cell cycle of human PDAC PANC-1 cells placed in the incubator (IC), at the bench (a) or inside the faraday cage treated with sham control (SC) or BT (b). The cells for the cell cycle analysis were collected immediately after 15 min treatment. C) The invasion of PANC-1 cells placed on the bench or inside the faraday cage. C) The invasion of human pancreatic cancer L3.7 cells placed on the bench or inside the faraday cage. The invasion assay was carried out at 48 hrs after 15 min treatment. Data are presented as mean±SD. *p<0.05, **p<0.01, ***p<0.001, ****p<0.0001.

FIG. 31. The effect of aluminum foil on the cell cycle and cell invasion of the PDAC cells treated with BT. A) Experimental setting of the aluminum foil study with cells either placed inside blue box without aluminum cover (left) or inside the box covered with aluminum foil (right). B) Cell cycle of PDAC PANC-1 cells placed in the incubator (IC), inside the box at the bench without covering with aluminum foil (a) or inside the box covered with aluminum foil treated with sham control (SC) or biofield therapy (BT, b). The cells for the cell cycle analysis were collected immediately after 15 min treatment. C) The invasion of PANC-1 cells placed in the box without aluminum foil on the bench or inside the box covering with aluminum foil. D) The invasion of human pancreatic cancer L3.7 cells placed inside the box without aluminum foil on the bench or inside the box covered with aluminum foil. The invasion assay was carried out at 48 hrs after 15 min treatment. Data are presented as mean±SD. *p<0.05, **p<0.01, ****p<0.0001.

FIG. 32. The effect of distance BT treatment on the invasiveness of the Panc-1 (A) and L3.7 cells (B). PANC-1 and L3.7 cells were treated by therapist 2 in the lab office (a), her apartment in Houston (b), and her home in California (c) for 15 min. Therapist was able to see the plates with cells via zoom. Immediately after treatment, cells were transferred to the incubator and continue incubated for 48 hrs prior to the cell invasion assay. Data are presented as mean±SD. ***p<0.001, ****p<0.0001.

FIG. 33. Experimental setup of entanglement study. BT therapist providing treatment to human pancreatic cancer cells. Cells designated for the measurement of tubulin are in the incubator equipped with the CytoSmart Lux microscope on the left. To the right of the BT therapist, the EVOS M7000 microscope captures images of Ca2+ or β-actin changes. Positioned behind the BT therapist is the EEG amplifier. Cells were enclosed and temperature regulated the CytoSmart microscope, so the proximity of the BT therapist is unlikely to influence outcomes and he remained blinded to cell type. The BT therapist's hands sometimes rested on the table and sometimes were held up. When resting on the table, the BT therapist's hand never touched the cell measurement devices.

FIG. 34. Cell measurements (tubulin, Ca2+, and β-actin) for the BT and sham-treatment control conditions (A) and significance of Granger causality between brain EEG signals in different frequency bands (theta, alpha, beta, gamma) and cell data (tubulin and Ca2+) (B). Values for each assay represent changes from the baseline obtained one minute before the initiation of treatment (A). The logarithmic scale indicates significance after FDR correction for multiple comparisons (electrodes in red are below 0.05 and significant, B).

FIG. 35. The invasiveness of PANC-1 cells (A) and liver metastasis of PANC-1 mouse orthotopic model (B) after being treated by the second biofield therapist. (A) The invasion of PANC-1 (a), COLO357 (b), and L3.7 (c) cells and migration of PANC-1 (d) and L3.7 (e) cells. (B). Percentage of liver nodules in the mice bearing PANC-1 tumor (a); Percent of liver mets (i.e., instances of metastasis or metastatic cancer) in the mice carrying different size of tumors (b); and Average of tumor weight of PANC-1 tumor without liver mets in SC and BT group. Data are presented as mean±SD. **p<0.01; ***p<0.001; ****p<0.0001.

FIG. 36. ATACseq analysis of the chromatin modification in PANC-1 cells after being treated for 15 min.

FIG. 37. FIG. 16. Mitochondrial membrane potential of PANC-1 cells after BT treatment for 15 min (A) or 30 min (B). Mitochondrial membrane potential was measured by JC-1 dye. The reduced ratio of red over green staining of JC-1 day suggested depolarized mitochondria.

FIG. 38. Transmission electron micrographs showing morphologic changes in L3.7 cells in the IC group (A and D), SC group (B and E), and BT group (c and f) after 15 min of exposure to treatment (samples were collected immediately after treatment). Note the presence of cytoskeleton elements (red arrows) in the IC and SC groups (D and E) and limited appearance of similar cytoskeleton structure in the BT group (F). Images A-C were taken at 5000×; images D-F were taken at 25,000×.

FIG. 39. The effect of BT on the invasiveness of PANC-1 cells after being treated by one therapy (J) or group therapists (Group) or the BT treated medium in the distance treatment setting. Data are presented as mean±SD (*p<0.05; **p<0.01; ****p<0.0001).

### DETAILED DESCRIPTION OF THE INVENTION

This patent describes systems and methodologies for healthy and/or homeostatic functionality of individual or particular biological entities, e.g., organisms (e.g., mice, humans), including groups and components thereof (e.g., cells, tissues, organs, organelles, organoids), such as those experiencing potentially deleterious cellular states or function, e.g., cancer and/or metastasis of cells. The systems and methods can include interactions and/or integrated effects or adjustments, for example, effects and/or changes within or of the entity, e.g., to entity state or function, and/or exterior to or not of the entity, e.g., to its microenvironment, which may result in a cascade of biological effects.

The systems and methodologies can effect, e.g., regulate, change or adjust, one or more aspects of the biophysics and/or bioelectricity of the entity, e.g., a patient, tissue, and/or cell-line, where the entity is experiencing or may experience attributes of dysfunction, poor health, and/or dysregulation, e.g., cancerous state(s) or activity(ies) of cells or attributes or indicators of cancer. The systems and methodologies can also or alternatively effect or result in regulation or adjustment of the structure and/or physiology of the entity, e.g., the patient, tissue and/or cell-line, where the entity is experiencing or may experience attributes of dysfunction, poor health, and/or dysregulation, e.g., cancerous state(s) or activity(ies) of cells or attributes or indicators of cancer. The systems and methods may change system-level patterns of biological functioning, for example, producing epigenetic shifts (as indicated, for example, by RNA methylation and/or histone modification), modifying protein and/or gene expression levels, and/or reprogramming cell signaling among entities.

The systems and methodologies described herein provide benefits for biological systems as a whole, e.g., tissues, organs, groups of cells or individuals, and rely on integrated effects as opposed, for example, to methods that may target or benefit a specific molecular site, reaction, or pathway or sets thereof. The systems and methodologies described herein may provide for sustained or regulated functioning and/or homeostasis of such systems. The systems and methodologies contemplate that forces, factors, and/or conditions common and/or external to such biological systems (e.g., a population of cells), including environment (e.g., medium), field (e.g., electromagnetic) and/or neighbor (e.g., signaling and network) effects, may influence and/or be integral to their functioning. The biological systems may comprise a plurality of biological entities, and the systems and methodologies may influence or effect one or more biological entities directly, e.g., by resulting in changes within or of the entity, or indirectly, e.g., by resulting in changes external to the biological entity or entities (e.g., in another entity and/or the environment of the entity, for example, the medium for culturing cells).

The systems and methodologies described herein can be used in the treatment of, or to devise and/or assess treatments for (e.g., evaluate and/or compare possible effectiveness of treatments or administrators of treatments, including without limitation evaluating or comparing individual therapists or groups of therapists, pharmaceutical, radiological treatments, and treatment devices) entities experiencing dysfunction, poor health, and/or dysregulation, e.g., organisms and/or cells experiencing cancerous-type growth and/or metastasis (e.g., cancer patients or cancer cells or cell lines). In one embodiment, the cells are human or murine cells, e.g., human or murine cancer cells. The systems and methodologies can be used in the treatment of or to devise and/or assess treatments (e.g., evaluate and/or compare possible effectiveness of treatments) for cancer patients and/or cancer cells or cell-lines, for example, pancreatic cancer patients and/or cells or cell-lines such as pancreatic ductal adenocarcinoma (PDAC) cells, including but not limited to cells from human cell lines, such as PANC-1, MiaPaca, COLO357, L3.7, and Capan2 cell lines, and cells from murine cell lines, such as Panc02 and KPCY. The systems and methodologies can promote healthy function and/or well-being, for example, of organelles, organoids, cells, tissues, and/or organisms, including groups thereof, in particular, mice and humans, by, for example, reducing growth and/or metastasis of cells, e.g., cancer cells or cells in or near a tumor.

Tumors are often considered to be local until metastasis occurs. However, increasing evidence points to the preexisting influence of multiple distal and proximal systems in the tumor microenvironment that affect tumor initiation and progression, including genome-wide epigenetic change preceding tumor formation and inflammatory responses to irritants, as discussed by Hill, W. et al., Nature 616:159-167 (2023), which is incorporated herein by reference. Cancer can be seen as cellular defections from the correct target morphology-an inability of the normal field of patterning information to orchestrate individual cells' activities toward the evolutionary success of the body as a whole. Put more simply, cancer may result from degraded cellular information exchange and bioelectrical decoherence.

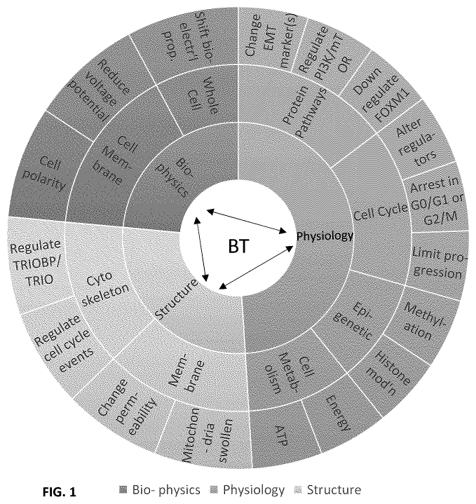

As shown in FIG. 1, factors that may be involved in the methods and systems described herein include, without limitation, factors characterized herein according to their biophysics, structure, and/or physiology. For example, membranes have both structural and biophysical aspects. A change in a structural aspect can be associated with, e.g., cause, a change in a biophysical aspect, and/or vice versa. For example, an increase in membrane permeability may result in a reduction in voltage potential. The biophysical realm, including (but not limited to) factors identified in FIG. 1 in the category of “biophysics,” appears to be a systemic, preexisting, dynamic influence on tumor formation. Factors characterized as physiological are also known to influence tumor formation, and they may include structural and/or biophysical aspects. A change in a physiological aspect can be associated with, e.g., cause, a change in a structural aspect, and/or vice versa. A change in a physiological aspect can be associated with, e.g., cause, a change in a biophysical aspect, and/or vice versa.

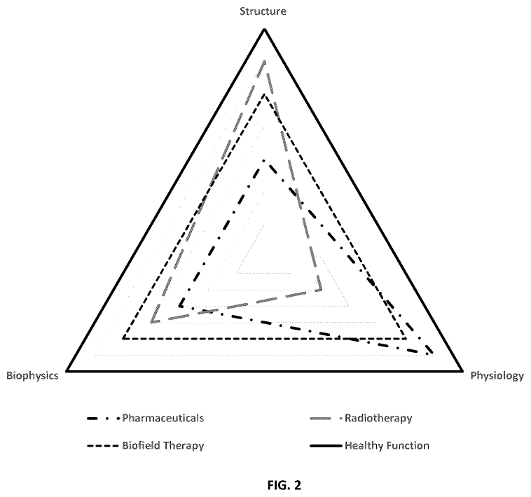

The systems and methodologies described herein can result in integrated and/or mutually beneficial effects on the biophysics, structure, and/or physiology of cells, including for example effects on the microenvironment, structure, bioelectricity and/or gene expression of cells. For example, as exemplified in FIG. 2, a biofield therapy may have beneficial effects on structural, biophysical, and physiological aspects of cells. In contrast, radiotherapy may improve the structural and biophysical properties of an organ (e.g., a pancreas) by ablation and death of cancerous cells (e.g., a tumor), but has deleterious effects on nearby healthy tissue. Also in contrast, pharmacological treatments may improve physiological functionality, but this may occur without positive or beneficial effects on cellular structure or biophysics.

In certain embodiments, the systems and methodologies effect (e.g., result in, directly or indirectly), multiple changes in factors contributing to and/or involved in (directly or indirectly) cellular homeostasis and/or maintenance or recovery of non-cancerous cell states, such as those shown, for example, in FIG. 1. The factors may be aspects of, e.g., components or properties of, cell state and/or function. The systems and methodologies may have epigenetic effects, such as changes in expression of genes and/or the transcriptome. The systems and methodologies can influence expression of protein for FOXM1 or affiliated proteins, for membrane ion channel genes, for cytoskeleton proteins, and/or for proteins involved in cell migration and invasion as well as intravasation.

In certain embodiments, the systems and methodologies described herein can effect changes in one, two, three, four, five or all of: cellular energy (e.g., mitochondrial state and/or function; ATP levels), cellular physiology (e.g., transcription regulators, FOXM1 and TRIM24, various protein kinases including the kinases associated with both glucose and glutamine energy metabolism, PLK1, and/or Ras associated pathways); cell cycle (e.g., the distribution among or rate of progression through cell cycle stages, p21, CDKs, cyclins); cell structure (e.g., cell junction and/or cytoskeleton organization); cell growth and/or metabolism (e.g., epidermal growth factor, EGF); cellular biophysics (e.g., membrane voltage potential and/or polarization of cells); the extracellular matrix (e.g., cell signaling), intracellular communication network (PDZ), and other aspects of cellular or molecular biology). One or more changes in factors that may be involved in cellular homeostasis and/or maintenance or recovery of non-cancerous cell states—e.g., cellular energy, physiology, cycle, structure, biophysics or bioelectricity, and the extracellular matrix—can result from an effected change, for example, in cellular energy, physiology, cycle, structure, biophysics, bioelectricity, and/or the extracellular matrix.

The systems and methodologies described herein may influence, e.g., result in beneficial changes in, cell physiology (e.g., regulation of FOXM1, regulation of the PI3K/mTOR pathway, changed EMT marker expression, delayed progression through the cell cycle and mobility), cell structure (e.g., regulating cytoskeleton organization, e.g., by regulating F-Actin, TRIO and TRIOBP as well as mitochondria function and structure), and/or cell biophysics (e.g., regulating membrane voltage potential, preventing or reversing depolarization of cells, and other shifts in bioelectrical properties of cells). The systems and methodologies described herein may influence, e.g., result in changes in, physiology of an organoid, organ, tissue, or organism. For example, the systems and methodologies can influence function of the autonomic nervous system (parasympathetic and sympathetic nervous system), immune system, brain, and/or heart (e.g., as measured, for example, by heart rate, heart rate variability, high frequency heart rate variability, ECG, brain waves, EEG, electrodermal activity, pupil dilation, etc.). The systems and methodologies may influence, e.g., result in changes in, physiology and/or cell structure as a result of changes in cell biophysics. The systems and methodologies may influence, e.g., result in changes in, cell biophysics as a result of changes in physiology and/or cell structure. The changes in cell physiology, structure, and/or biophysics can result in decreased growth and/or metastasis of cells, e.g., cancer cells.

Definitions are provided as follows:

A “living entity” or “biological entity” as used herein refers broadly to any individual that is alive and any group or component thereof, including for example cells, single and multi-celled organisms (e.g., animals, mammals, mice, humans), organoids, organelles, embryos, tissues, organs, tumors, growths existing in any form, including in vitro and in vivo.

The term “growth” as used herein refers broadly to individual and/or population growth, for example, an increase in mass of an individual cell or tissue and/or increased viability, proliferation, and/or reproduction of cells.

The terms “regulate” and/or “regulation” as used herein refer to adjustment, maintenance or control of a process or state, including but not limited to prevention or reduction of excursions from a desirable state.

The term “homeostasis” as used herein refers to any self-regulating process by which a biological system tends to maintain stability, e.g., a tendency toward a relatively stable state or process.

The terms “metastasis” and “metastatic potential” as used herein refer to the actual or potential development of the ability of cancerous cells to separate, migrate and/or invade (e.g., other tissues or organs), which can, for example, serve to establish secondary malignant growths at a distance from a primary site of cancer.

The terms “energy therapy” and “energy healing” as used herein refer to any therapy that changes the energy balance or state of a living entity, including for example changes in the bioelectrical, biophysical and/or metabolic balance or state. Energy therapy/healing includes (but is not limited to) techniques that involve so-called “channeling” of energy, e.g., healing energy, through a practitioner into an entity, which has been used to treat a wide variety of ailments and health problems, either alone or in combination with other medical treatments, for example, administration of pharmaceuticals and/or radiation. In general, energy therapy restores or creates a beneficial state and/or more healthy functioning of the entity of the therapy.

The term “biofield therapy” or “BT” as used herein is a form of energy therapy and refers broadly to any and all therapeutic approaches that involve the use of a body's (or bodies') energy field(s) (biofield) for therapeutic benefit and encompasses devices, e.g., devices that mimic fields and/or information emitted by or exchanged with people (e.g., healers and/or healees) when administering or participating in biofield therapy. Biofield therapies are used today in the Veterans Administration and across dozens of major hospitals with tens of thousands of practitioners in the US alone, and has been demonstrated to result in changes in the biophysical state and biochemical signaling of recipient cells.

As used herein, and unless otherwise specified, “to treat” and “treatment” is not limited to treatment of a disease or condition. For the sake of clarity, “to treat” or “treatment” includes but is not limited to use of the described effects and mechanisms to eliminate or reduce the effects of a disease or condition, to restore healthy and/or homeostatic functioning, for general health and well-being, and/or for purposes of prophylaxis or prevention of disease, and encompasses use of the described effects and mechanisms with or without diagnosis of a disease state or condition.

### Indicators of Efficacy

The systems and methodologies describe herein can provide an indicator of efficacy of a treatment, including for example and without limitation, a form or manner of treatment, an administrator or type of administrator of treatment, a biofield therapy, a pharmacological treatment, a radiotherapy treatment, or any combination thereof. The indicator of efficacy can be used, for example, as a diagnostic for the effects or effectiveness of a biofield therapy or biofield therapist or group of therapists. The indicator of efficacy can be used to determine whether an entity, e.g., an entity characterized by a cancer, e.g., pancreatic cancer and/or a cancer characterized by over-expression of FOXM1, is likely to be therapeutically involved in or responsive to a treatment, e.g., a biofield therapy and/or administrator of biofield therapy.

The indicator can comprise or be based upon, for example, one or more measurements indicative of change or absence of change in physiology of an administrator of treatment, e.g., a BT practitioner. For example, the indicator can be based on one or more measurements of the autonomic nervous system (parasympathetic and sympathetic nervous system), brain, and/or heart (e.g., heart rate, heart rate variability, high frequency heart rate variability, ECG, brain wave frequency or spectrum, EEG, electrodermal activity, pupil dilation), for example, as described in Cohen, L., et al., Examining the effects of biofield therapy through simultaneous assessment of electrophysiological and cellular outcomes, Nature Scientific Reports 14:29221 (2024) (https://doi.org/10.1038/s41598-024-79617-3) (“Cohen et al. 2024”), which is incorporated by reference herein in its entirety.

The indicator can comprise or be based upon, for example, one or more measurements indicative of change or absence of change in the recipient of a treatment, and may include one or more factors contributing to and/or involved in cellular homeostasis and/or maintenance or recovery of non-cancerous cell states, such as those shown, for example, in FIG. 1. For example, the indicator can comprise or be based upon measurements of physiology, biological function, physical structure, and/or biophysical state of an entity of treatment, including, for example, those described in Cohen et al. 2024. The indicator can be selected or determined based on an initial state of or measurement for an entity of treatment.

The indicator can be based upon one or more correlation between the physiology of an administrator, as noted above, or the recipient of a treatment, as noted above.

In one embodiment, the indicator of efficacy comprises or is based at least in part upon measurement of one, two, three, four, five, six, seven, eight, nine, ten or more proteins, for example, one or more measurements of: calcium, FOXM1, pAKT, ALKBH5, cyclin B1, CDK1, PI3K/mTOR, P21, TRIM24, EMT marker expression, E-cadherin, N-cadherin, Twist, and/or CD44. In one embodiment, the indicator of efficacy comprises or is based upon at least one or at least two measurements of FOXM1. For example, the indicator of efficacy can comprise or be based upon measurement of FOXM1 and selected based on a pre-treatment or initial measurement of FOXM1 indicative of over-expression of FOXM1 and reduction of FOXM1 after treatment. Any metric or measurement of FOXM1 expression can be used as an indicator of efficacy.

In one embodiment, the indicator of efficacy comprises or is based at least in part upon measurement of cell cycle, for example, number or proportion of cells in a cell cycle phase, e.g., in G0/G1 and/or G2/M. In a preferred embodiment, the indicator of efficacy comprises or is based upon at least one or at least two measurements of the proportion of cells in G0 and/or G1. In one embodiment, the indicator of efficacy comprises or is based at least in part upon measurement of cellular structure, for example, state of the cytoskeleton (e.g., tubulin), cytoskeleton protein, mitochondria, and/or F-actin, TRIO, TRIOBP. In one embodiment, the indicator of efficacy comprises or is based at least upon at least one or at least two measurements of mitochondrial state, for example, morphology (e.g., shape, swelling), order, membrane (e.g., voltage potential, polarization), energy (e.g., ATP, usage). In one embodiment, the indicator of efficacy comprises or is based at least in part upon at least one or at least two measurements of TRIOBP.

In one embodiment, the indicator of efficacy comprises or is based at least in part upon measurement of biophysical or bioelectrical state, for example, voltage potential or polarization of a membrane, e.g., cellular, nuclear, and/or mitochondrial membrane. In a preferred embodiment, the indicator of efficacy comprises or is based upon at least one or at least two measurements of cell membrane voltage potential.

The indicator of efficacy can comprise or be based at least in part upon one or more measurements of two, three, four, five, six, seven, eight, nine, ten or more aspects of an entity, e.g., protein levels or expression, cell cycle, cellular structure, and/or biophysical state, for example, as identified in the Figures and Examples provided herein.

A measurement can be made or indicative of status after treatment. Two measurements can include, for example, a first measurement made or indicative of status before treatment and a second measurement made or indicative of status after treatment. In one embodiment, the first and second measurements are measurements of the same aspect, feature, metric, or factor, e.g., the same protein, same cell cycle metric, same cellular structure measurement, and/or the same biophysical state.

### Biofield Therapy

Biofield treatment methods and/or devices have lesser toxic or deleterious effects than radiotherapy and/or pharmacological treatments. They may therefore be more acceptable and/or tolerable forms of treatment than radiotherapy and/or pharmacological treatments. They may further benefit from faster times to approval, given their lower toxicity and lesser safety concerns.

The systems and methodologies described herein may integrate and/or relate scientific aspects from fields of study of physics including biophysics and/or bioelectricity, consciousness including healing, biology including quantum biology and pharmacology, and medicine/health including conventional and nonconventional treatments. The systems and methodologies may occur as the result of interaction and/or exchange of conscious human intention with energy (e.g., electrical, light, biophoton, and/or magnetic fields), information, and/or biology. The effect and/or mechanisms may occur as the result of a field or fields, for example, information and/or energy fields, including (but not limited to) in chemical and/or physical environments. In one embodiment, the field or fields includes a dynamic, spatial field that contains information. A field may be a massless field, possibly electromagnetic but not necessarily electromagnetic, that surrounds, permeates and/or influences biological entities. A field may be self-organizing and/or may comprise or provide a template or blueprint. A field may influence a biological entity and/or features or aspects of the biological entity (e.g., a tumor, autonomic nervous system (parasympathetic and sympathetic nervous system), brain, brain function, heart, heart rate, metastatic processes, metabolism, gene and protein function, and more) towards increased order and/or homeostasis. Energy and/or information may involve or be conveyed by one or more of: quantum entanglement; extracellular chemical diffusion or propagation; frequency, resonance, and/or vibration (e.g., longitudinal waves); electromagnetism (EMF); and sound, light, and/or dark matter. A field, energy and/or information may be, for example, referred to as healing energy, subtle energy, elan vital, prana, qi, chi, vital force, and/or biofields.

The changes may occur from the actions or intentions of a person, e.g., a BT therapist or group of therapists, or a patient. The changes may occur from the operation of a device, e.g., a field or information generator. The field or information generator may be, for example, a device that generates or emits electromagnetic energy, light, and/or sound. The actions, intentions, and/or operation of the device may facilitate exchange or flow of energy or information among or between entities, for example, one or more organs, organelles, organoids (e.g., pancreatic cancer organoids), cells, tissues, and/or organisms, in particular, mice and/or humans, where such entities are, for example, in diads, groups, or collectives. The actions, intentions, and/or operation of the device may influence or manipulate fields, e.g., energy or information fields. The actions, intentions, and/or operation of the device may be proximal to the entity. The actions, intentions, and/or operation of the device may be precedent to the effect.

The BT effects and mechanisms can be evidenced by correlation between the actions, intentions, and/or operation of the practitioner or device and observed changes, outcomes, or states of one or more entities, as described, for example, in Cohen et al. 2024. The entity can be any biological entity, e.g., an organism (e.g., a mouse, human), and/or can comprise cells, organs, organelles, organoids, tissue, tumor, abnormal growths, and the like, or groups thereof. In one embodiment, a method of treatment includes the actions, intentions, and/or operation of the device that correlates with the observed changes, outcomes, or states of the entity. The method can be transpersonal, for example, between a therapist or group of therapists and an entity. In one embodiment, the method is a transpersonal consciousness-mediated therapeutic modality. In one embodiment, the methods consist of administering a form of energy or biofield therapy, for example, Bengston healing, Healing Touch, Reiki, Therapeutic Touch, or External Qigong. In one embodiment, the methods comprise administration a form of energy or biofield therapy, for example, Bengston healing, Healing Touch, Reiki, Therapeutic Touch or External Qigong.

In one aspect, the effects of a person administering energy therapy or a device used to administer energy therapy can be assessed, quantified, evaluated or otherwise indicated based on one or more indicators of efficacy. For example, prior and subsequent to the administration of a biofield treatment, levels of FOXM1 in the cells of an entity experiencing a state of cancer are measured. If levels of FOXM1 prior to treatment are indicative of overexpression of FOXM1, one or more measurements of the expression of FOXM1 post-treatment can be used as an indicator of efficacy. For example, if post-treatment measurements of FOXM1 are significantly lower than pre-treatment measurements, the treatment (e.g., the person or device administering the treatment) may be deemed efficacious.

### Integration of Effects

In general, changes in structural, biophysical, and physiological aspects of cells may have integrated effects on cellular health and, in particular, cancerous function and/or state of cells.

The physiological realm, including (but not limited to) factors identified in FIG. 2 in the category of “physiology,” is generally recognized as an important context for understanding cancer. For example, EMT markers are known to be indicative of cancer and cancer metastasis. The structural realm, including (but not limited to) factors identified in FIG. 1 in the category of “structure,” is known to be involved in regulation of the cell cycle, cell growth and cell metastasis, a recognized feature of cancer. The biophysical realm, including (but not limited to) factors identified in FIG. 1 in the category of “bio-physics,” includes feature of cell polarization. Cancerous cells appear to be typically depolarized.

Associated changes in structural, biophysical, and physiological aspects of cells, and systems and methods that employ such associated changes, may have particular benefits for the treatment of cancer, e.g., pancreatic cancer. Cancer may be a reversible physiologic state of a multicellular dynamical system. In this regard, consideration of multiple aspects may be integral to understanding carcinogenic mechanisms. Cancer may be the result of a dysfunction in the cellular and tissue microenvironment resulting from the combined effects of structural, biophysical, and physiological factors. We consider several such combinations as follows.

### Cell Voltage and Cancer.

Cancer cells are known to possess distinct bioelectrical properties. Endogenous spatial-temporal differences in resting cell membrane potential and bioelectric gradients are an important layer of the dysregulation of cell-cell interactions that leads to cancer (Chernet B, Levin M., J Clin Exp Oncol. 2013; Suppl 1). Cell membrane voltage potential (Vm), the voltage across the plasma membrane that arises because of the presence of different ion channels/transporters with specific ion selectivity and permeability, is implicated in cancer cell proliferation and metastasis. In addition, abnormal depolarization of resting membrane voltage potential (Vm) has been considered as a convenient marker for neoplasia and activates a metastatic phenotype in genetically normal cells in vivo. Vm change triggers metastatic behaviors at considerable distance, mediated by transcriptional and epigenetic effects of electrically-modulated flows of serotonin and butyrate. See Yang M, Brackenbury W J. Front Physiol. 2013; 4:185; Chernet B T, Levin M., Dis Model Mech. May 2013; 6(3):595-607; Levin M., Mol Biol Cell. Dec. 1 2014; 25(24):3835-50.

Cell membrane potential is mediated through different concentration of ions and ion channels that play an important role in oncogenesis (Becchetti A., Am J Physiol Cell Physiol. August 2011; 301(2):C255-65). Electrophysiological analyses in many cancer cell types have revealed a depolarized Vm that favors cell proliferation and migration. The range of the Vm in the proliferating cells including tumor and electrically excited normal cells is above −50 mV, whereas terminally differentiated cells, such as neurons and smooth muscles, have Vm around −90 mV, suggesting the Vm is functionally instructive for cell development. Human breast cancer MCF-7 cells have Vm ranging between −9 and −30 mV depending on the phase of cell cycle, which is still higher than that of immortalized MCF-10A cells (−40 to −58 mV). However, transient hyperpolarization promotes the migration and metastasis of breast cancer, primarily mediated by voltage gated potassium channel. See Yang M, et al., Front Physiol. 2013; 4:185; Levin M., Trends Cell Biol. June 2007; 17(6):261-70; Yang M, et al. Breast Cancer Res Treat July 2012; 134(2):603-15; Marino A A, et al. Tumour Biol. 1994; 15(3):147-52; Fraser S P, et al. Clinical cancer research. Aug. 1 2005; 11(15):5381-9; Payne S L, et al. EBioMedicine. January 2022; 75:103767; Hofschroer V, et al. Front Pharmacol. 2020; 11:586599.

### Ion Channel and Metastasis.

Ion channels are a major class of membrane proteins that can sense and modify properties of the tumor microenvironment and transduce signaling cascades triggered by its constituents either by alteration of conductivity of the cell membrane or non-conductive mechanisms. They are expressed in every cell and exert cell-specific functions and housekeeping functions including generating and regulating the membrane potential which, in turn, is a prerequisite for many processes such as Ca2+ signaling, mitochondrial function, and interaction among tumor and stromal cells within tumor microenviroment (Djamgoz M B, et al. Philos Trans R Soc Lond B Biol Sci. Mar. 19 2014; 369(1638):20130092). Among various ion channels, voltage gated sodium channel (Nav) and potassium channel (Kv) are known cell membrane voltage regulators and are involved in cancer cell proliferation and metastasis (Djamgoz MBA, et al. Cancers (Basel). Oct. 28 2019; 11(11); Zuniga L, et al., Onco Targets Ther. 2022; 15:783-797). Potassium channel activation can cause hyperpolarization, whereas opening of sodium or chloride channels can cause depolarization. Nav1.5 has been reported to be associated with increased metastatic potential of breast cancer cells51. Kv channel contains 12 subfamily of 40 genes and plays a major role in repolarizing the resting cell membrane potential in excited cells (Wulff H, et al. Nature reviews Drug discovery. December 2009; 8(12):982-1001; Feske S, et al. Annu Rev Immunol. 2015; 33:291-353). In nonexcitable proliferating cells, Kv channels play an important role in cell proliferation, Ca2+ signaling, migration, and cell volume regulation (Chesnokov M S, et al., Front Oncol. 2021; 11:696532.). Kv1.3 channel is overexpressed in several PDAC cell lines, and its overexpression is associated with increased proliferation and cancer cell survival.

Targeting PDAC cells with a Kv1.3 inhibitor (clofazimine) induces apoptosis and slows down the growth of orthotopically transplanted PDAC cells (Lobikin M, et al., Phys Biol. 2012; 9(6):065002). Kv1.3 is also expressed in the mitochondria (mitoKV1.3), where it regulates apoptosis in PDAC cells (Leanza L, et al., Cancer cell. Apr. 10 2017; 31(4):516-531 e10). Additionally, KCa3.1, a calcium activated potassium channel, is functionally expressed in pancreatic ducts and are part of the transepithelial ion and fluid transport machinery (Hayashi M, et al. Am J Physiol Cell Physiol. Jul. 15 2012; 303(2):C151-9; Wang J, et al, Am J Physiol Cell Physiol. Apr. 1 2013; 304(7):C673-84). KCa3.1, as the first K+ channels, is highly overexpressed in primary pancreatic cancer samples and is functional in pancreatic cancer cell lines (Zaccagnino A, et al., Eur Biophys J. October 2016; 45(7):749-763; Jager H, et al., Mol Pharmacol. March 2004; 65(3):630-8; Jiang S, et al., Biochem Biophys Res Commun. Dec. 9 2017; 494(1-2):113-119; Jiang S H, et al., Gut. November 2019; 68(11):1994-2006). Higher KCa3.1 channel expression is associated with poor patient survival in PDAC and other cancer types (Faouzi M, et al., Oncotarget. Jun. 14 2016; 7(24):36419-36435; Bulk E, et al., Int J Cancer. Sep. 15 2015; 137(6):1306-17). KCa3.1-mediated K+ efflux is necessary for volume dynamics during the cell cycle63 and migration. Thus, KCa3.1 can promote tumor progression by modulating cell proliferation, migration, and invasion. Finally, similar to Kv1.3, KCa3.1 channels are also expressed in the inner membrane of mitochondria (IMM) in normal and PDAC cells64 and regulate metabolic activity of mitochondria, potentially by modulating mitochondrial membrane potentials.

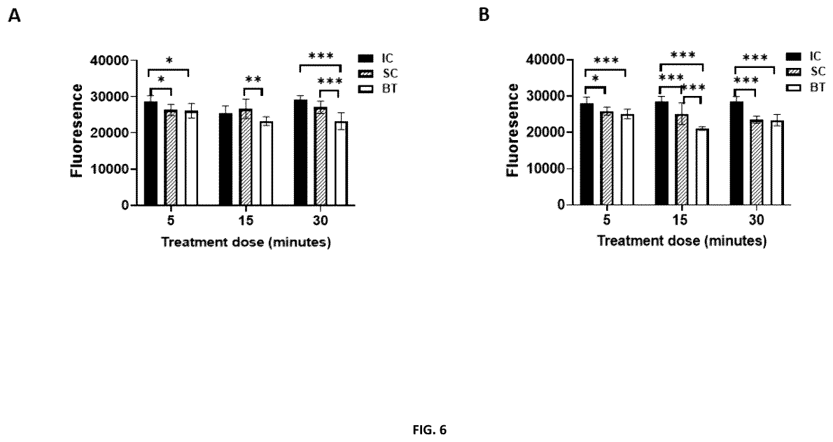

As described below in the Examples, BT treatment significantly reduced the invasion of Panc-1, L3.7, COLO357 (human), and KPC (murine) cells and modulated the expression of Na+ and K+ channel genes (FIG. 6). Our data indicated that the expression of Nav1.5 and Kv1.3 genes were modulated by BT treatment in a temporal manner whereas KCa3.1 gene expression was downregulated in BT treated samples, which could be important for BT-induced anti-metastatic activity.

### Cell Volumes Regulation, Morphology and Metastasis.

Cancer metastasis, the result of movement of cancer cells from the primary site to a distant site or organ, is a highly coordinated multistep process that involves the stroma, blood vessels, and the cytoskeleton. Successful metastasis depends on invasion, migration, extravasation, anoikis, and angiogenesis. Invasion is a localized step that occurs in the tumor-host interface, where the tumor and stromal cells exchange enzymes and cytokines that modulate local extra cellular membrane (ECM) and stimulate cell migration (de Visser K E, Joyce J A. Cancer cell. Mar. 13 2023; 41(3):374-403).

One of the major events for the metastasis is reorganization of cytoskeleton and morphology to allow the cells to successfully execute the invasion and extravasation steps. Modification of cell morphology, i.e., cell shrinkage in glioma tumor, enhances the migration and extravasation process of glioma cells (Sontheimer H. Exp Biol Med (Maywood). July 2008; 233(7):779-91). Cell shrinkage requires cytoplastic water content to be reduced which is likely mediated by the electrochemical driving force for K+ and Cl−. Hypertonic cell shrinkage can potentially inhibit local volume gain which facilitates cell migration67. The efflux of Cl− and K+ through ion channels or transporters provides the driving force for the water to leave the cells, which has been proposed to happen in the migrating cells.

It is well established that the cell migration is regulated by water and ions (Schwab A, et al., Physiol Rev. October 2012; 92(4):1865-913). Ca2+-sensitive K+ channels, especially KCa3.1, are activated locally at the rear of migrating MDCK-F cells, which may reflect its contribution to the decreased cell volume in the rear retraction cell migration process (Schwab A, et al., Pflugers Arch. September 1995; 430(5):802-7). The alterations in cell volume can be seen by a deformation of the cytoskeleton. The cytoskeleton could play a regulatory role by altering the availability of signal transducers or by being an integral part of the signal transduction system. A decrease in extracellular osmolality (hypotonic) in G6 glial cells leads to the shift of actin towards it polymerized F-actin form (Persinger M A, et al., Int J Yoga. July 2012; 5(2):140-5). Bioelectric changes mediated through ion channels leads to alteration of osmolality, resulting in cytoskeleton changes and influencing the migration potential of the cells.

In addition, cytoskeletal marker shifts suggest that microtubules may represent biophysical signaling systems. These ferromagnetic lattices align in parallel to strong magnetic fields, accounted for by single unpaired electrons per tubulin and they are involved in intracellular transport and chromosome separation in mitosis and meiosis. Microtubules are also non-linear oscillating structures generating an electromagnetic field in living cells. Structural and functional aspects of microtubules, e.g., their role in cell division and/or quantum vibrations, may influence cellular communication. Microtubules may be related to cancer as impairment of quantum coherence and/or entanglement among microtubule-based mitotic spindles and centrioles can result in abnormal distribution of chromosomes, abnormal differentiation, and uncontrolled growth. Beta Actin (cytoskeletal) fibers also generate spatially distributed endogenous bioelectric fields, another example of a structural aspect influencing a biophysical aspect. Cytoskeleton activity is implicated in the healing treatments, as is activity in Viafluo (tubulin, cytoskeletal) and Fluo4 (voltage).

### Mitochondria, Metabolism, and Metastasis.

Mitochondria play an essential role in fueling cells by generating energy in the form of ATP and regulate intracellular energy metabolism by oxidative phosphorylation (OXPHOS). Mitochondrial ion transport mechanisms also play a crucial role in regulating mitochondrial bioenergetics through changes in matrix Ca2+ levels that, in turn, affect mitochondrial respiration and ATP generation under physiological conditions (Kaufman R J, et al., Biochim Biophys Acta. October 2014; 1843(10):2233-9).

Under physiological conditions, ion transport (i.e., Na+, K+, Mg2+, H+, and Ca2+) through the IMM regulates mitochondrial matrix volume. The mitochondrial K+/H+ exchanger (KHE) is an IMM protein that plays a critical role in regulating the transport of K+ and may thus regulate mitochondrial volume and metabolism. K+ and Ca2+ have been identified as the primary drivers of mitochondria matrix volume changes. The main mechanism of mitochondrial swelling involves the opening of non-selective channels, known as permeability transition pores (PTPs) in low-conductance (physiological) and high-conductance (pathological) modes in the inner mitochondrial membrane. Depolarization of mitochondrial potential can contribute to mitochondrial swelling. An extensive increase of the matrix volume or matrix swelling compromises mitochondrial metabolism and function and can lead to cell death. See Douglas M G, et al., J Biol Chem. Sep. 10 1974; 249(17):5464-71; Halestrap A P, et al., Biochem J. Jun. 15 1986; 236(3):779-87; Chapa-Dubocq X R, et al., Antioxidants (Basel). Jul. 28 2023; 12(8); Kwong J Q, et al., Cell metabolism. Feb. 3 2015; 21(2):206-214.

### Immunity and Cancer.

The tumor microenvironment (TME) contributes to tumor initiation, progression, and response to therapy. Immune therapy, e.g., using immune checkpoint inhibitors for treatment of various malignancies, implicates the immune system as an important factor in cancer and cancer treatment (Shalapour S, et al., The Journal of clinical investigation. September 2015; 125(9):3347-55). Crosstalk between cancer cells and immune cells is one of the major hallmarks of cancer (Hanahan D, et al., Cell. Mar. 4 2011; 144(5):646-74).

Owing to their tremendous diversity and plasticity, immune cells exert multifaceted effects in tumor-bearing hosts, with different immune cells playing different roles. The composition and function of immune cells in tumors also greatly varies between and within the tumor types. Immunosuppressive TME that prevents effector T cells infiltration, activation, and elimination of tumor cells is considered a major barrier for the success of cancer treatments, including immune therapy, especially in PDAC (Dudley M E, et al., Journal of clinical oncology. Jun. 10 2013; 31(17):2152-9; Schumacher T N, et al., Cancer cell. Jan. 12 2015; 27(1):12-4). Based on the relative proportion of CD3+ and CD8+ cells relative to all cells in the tumor, PDAC is often ranked among the “coldest” human tumors (Galon J, Bruni D. Nature reviews Drug discovery. March 2019; 18(3):197-218; Maleki Vareki S. J Immunother Cancer. Dec. 27 2018; 6(1):157).

Targeting CTL-4 may enhance antitumor immunity and lead to tumor regression in multiple cancers including PDAC (Babamohamadi M, et al., Cell Death Dis. Jan. 8 2024; 15(1):17; Lisi L, et al., Pharmacol Res. January 2022; 175:105997; Ni R, et al., Biomed Pharmacother. October 2024; 179:117430). Additionally, inhibition of B-cell infiltration into the tumor by blocking chemokine CXCL13, inhibition of B-cell activity using a Bruton tyrosine kinase (BTK) inhibitor, or depletion of B cells using a specific mAb can significantly reduce tumor progression (Chen S S, et al., Leukemia. April 2016; 30(4):833-43). PDAC cell lines, derived from primary pancreatic carcinomas of transgenic KrasG12D;Pdx Cre mice harboring p16 Ink4 a or Trp53 deletion demonstrate that BTK regulates B-cell and macrophage-mediated T-cell suppression in PDAC development (Ireland L, et al., Cancer research. Dec. 1 2016; 76(23):6851-6863), suggesting tumor infiltrating B-cells are also important for PDAC.

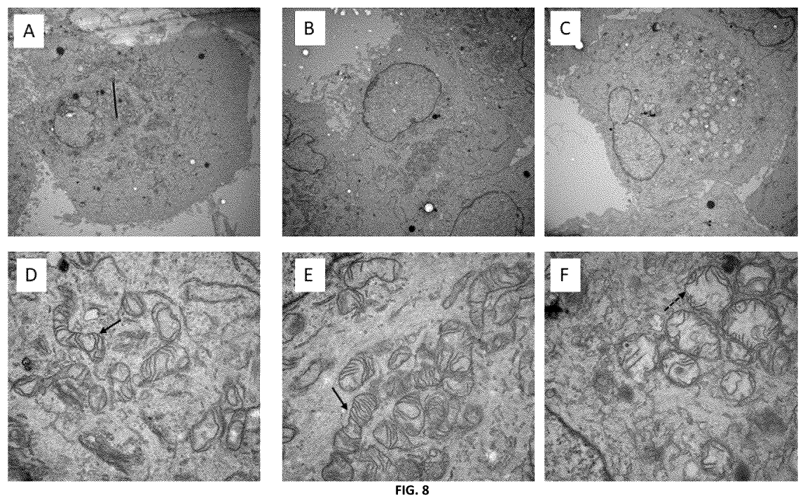

As provided in the Examples, our data showed that BT treatment significantly decreased B-cells and increased the CD8+-T cells (FIG. 8), indicating that BT modulated the immune TME and may contribute to the anti-metastatic potential of BT in PDAC.

### Metabolism in Cancer and Immune Cells.

Cancer cells are metabolically distinct from resting cells or tissues (Warburg Effect). Even in the presence of oxygen, cancer cells reprogram their glucose metabolism and their energy production by limiting their energy metabolism largely to glycolysis (Liberti M V, et al., Trends in biochemical sciences. March 2016; 41(3):211-218). Glycolytic fueling has been shown to be associated with activated oncogenes, such as RAS and MYC, and mutant tumor suppressors (TP53) (Jones R G, et al., Genes & development. Mar. 1 2009; 23(5):537-48; Wise D R, et al., Proc Natl Acad Sci USA. Dec. 2, 2008; 105(48):18782-7.), which then promote cell proliferation, avoidance of cytostatic controls, and reduction of apoptosis. Reprogrammed energy metabolism is recognized as an emerging cancer hallmark (Hanahan D, et al., Cell. Mar. 4 2011; 144(5):646-74).

The rewired cellular metabolism is not only a hallmark of tumors, but a feature of normal proliferating immune cells, such as T-cells (Andrejeva G, et al., Cell metabolism. Jul. 5 2017; 26(1):49-70). For example, activated T cells and leukemic T cells had a significantly higher rate of glucose metabolism (Kishton R J, et al., Cell metabolism. Apr. 12 2016; 23(4):649-62). T cell tumor trafficking and T cell cytotoxicity are impaired in glycolytic tumor and glycolysis inhibition enhanced antitumor activity of tumor-reactive T-cells (Cascone T, et al., Cell metabolism. May 1 2018; 27(5):977-987.e4), suggesting metabolic programing of tumors could potentially affect the function of immune cells.

### Brief Summaries from Examples

As further demonstrated and explained in the example(s) provided herein, systems and methods having the physiological, structural, and biophysical effects and mechanisms identified herein (e.g., biofield therapy) can inhibit primary tumor growth and reduce metastatic potential of cancerous cells.

As described in Example 1, BT has antitumor and anti-metastatic effects in multiple pancreatic cancer cell lines and two PDAC models.

FOXM1 is one of the mechanisms responsible for BT-induced anti-metastatic activity. The methods can reduce FOXM1, for example, reducing its expression in PANC-1 and L3.7 cells. The methods can modify metabolic activity, growth, and invasiveness of PDAC cells through downregulation of FOXM1. Methods that down-regulate FOXM1 can reduce the invasiveness of cancerous cells, e.g., PDAC cells. The down-regulation of

FOXM1 can be correlated with the reduction in invasiveness of cancerous cells, e.g., PDAC cells. The effect of BT on FOXM1 is highly positively correlated with the effect of BT on invasiveness. This means the greater the effect of BT in reducing FOXM1 the greater the reduction in invasiveness, e.g., relative to a lesser reduction of FOXM1 by BT.

The methods can change cell membrane potential. BT alters cell membrane voltage potentials and modulates the ultrastructure of the mitochondria. Additionally, BT treatment significantly alters gene expression of voltage gated Na+ and K+ channels, which are main regulators for the cell membrane voltage potentials. Biofield therapy can inhibit the growth and/or invasiveness of cancer cells, e.g., pancreatic cancer cells, and inhibition of growth and/or invasiveness can be mediated in part through modification of the cell cycle, alternations in cell voltage potentials, down regulation of PI3K/Akt pathways, and/or downregulation (reduction) of FOXM1.

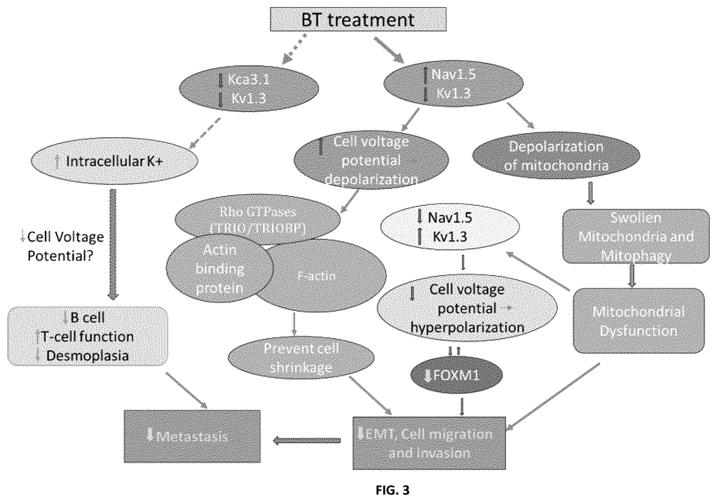

As described in Example 2, we assessed various steps of the metastatic process that BT acts upon using various in vitro cells and in vivo PDAC models. We identified a variety of potentially interacting aspects of bioelectricity and cell behavior, as shown in FIG. 3, including cell and mitochondrial membrane potential, mitochondria volume, voltage gated ion channels, FOXM1 signaling, and cytoskeleton changes (such as those identified in FIG. 1). We also identified effects of BT on the TME.

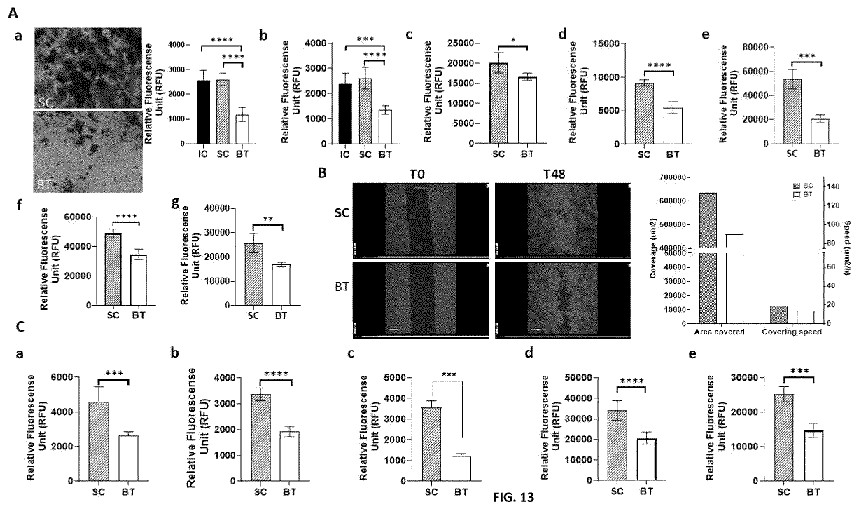

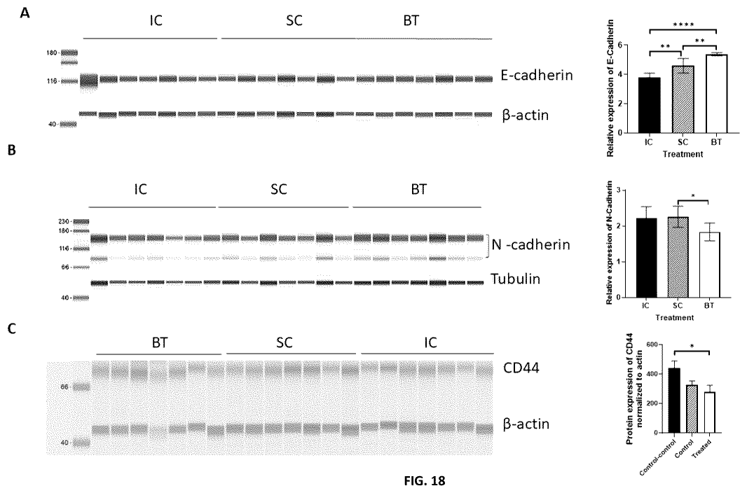

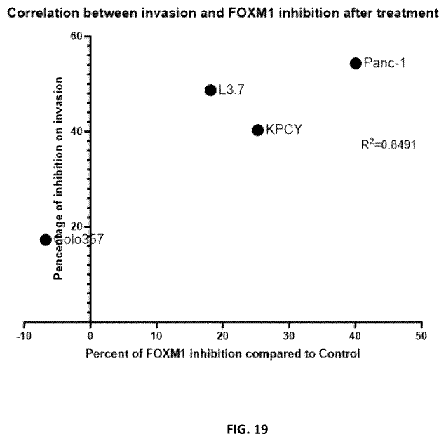

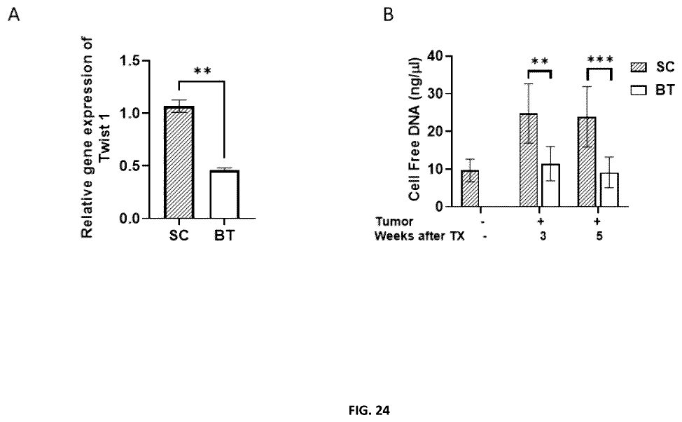

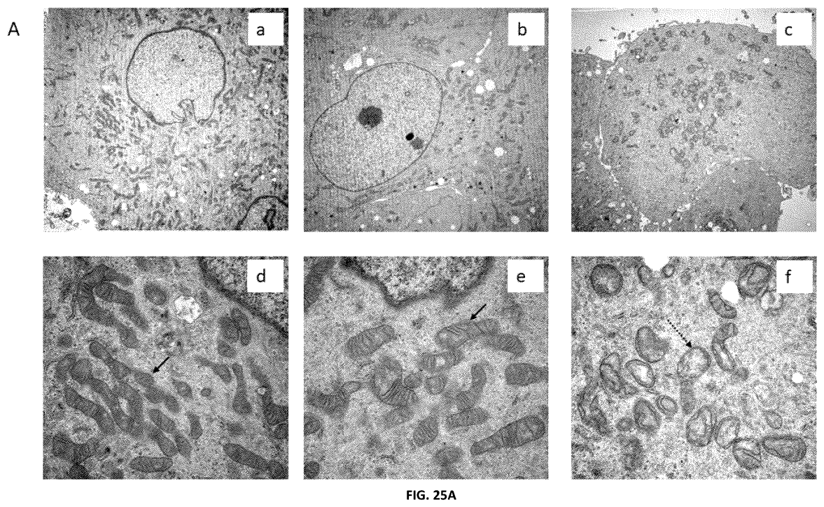

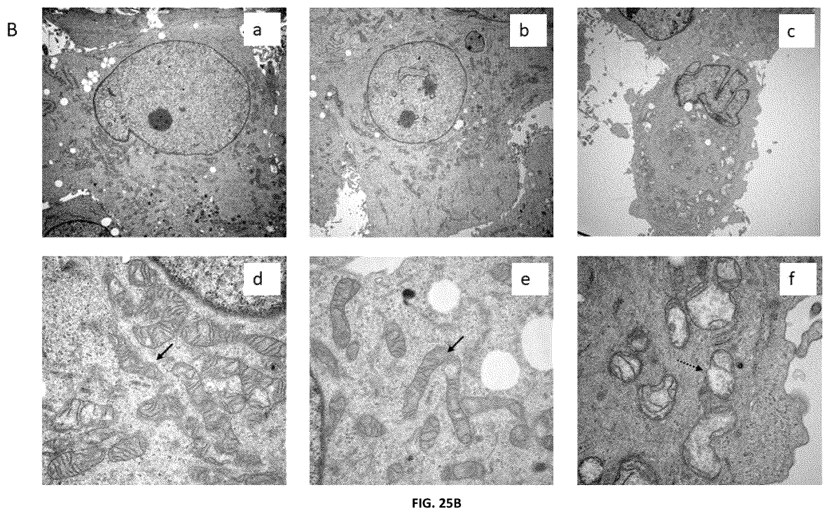

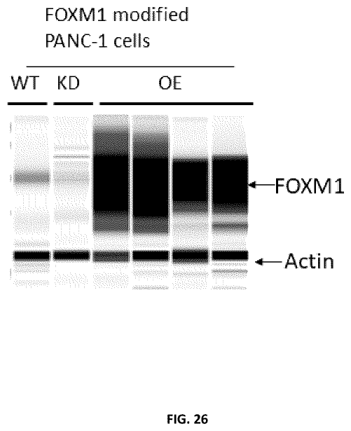

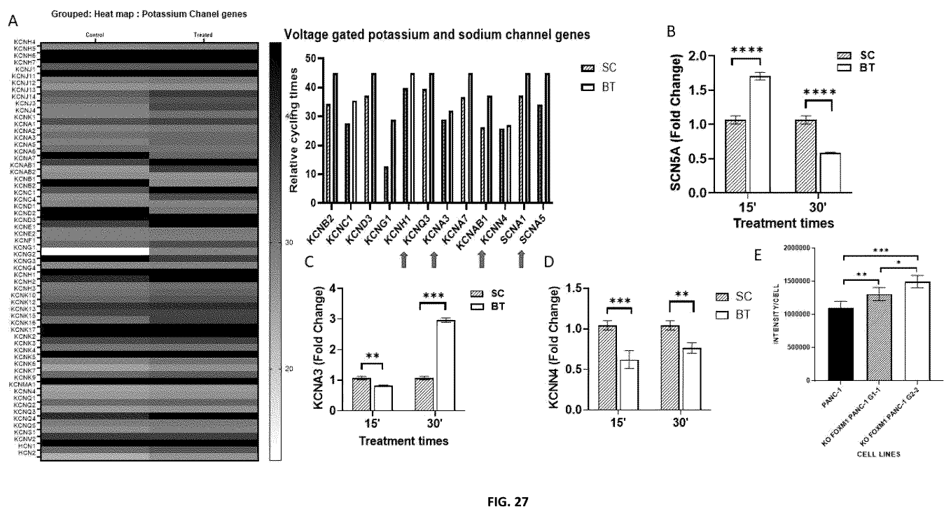

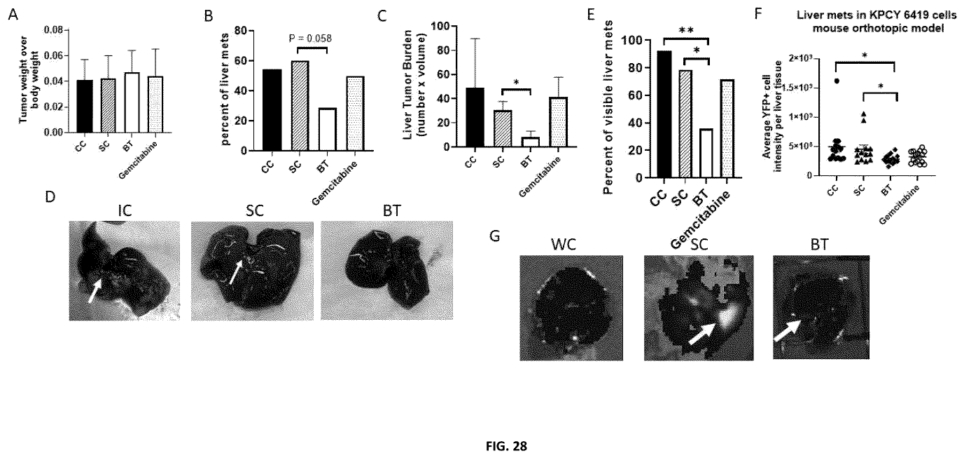

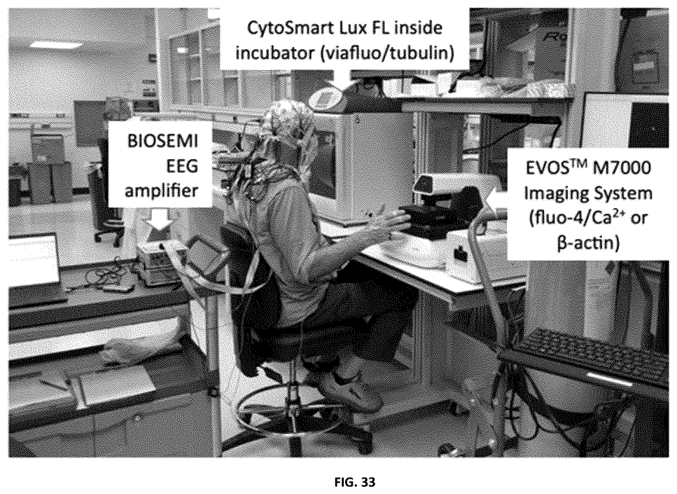

BT treatment significantly reduced the invasion and migration of various human and mouse PDAC cells in a number of repeated studies (FIG. 13). BT impacted EMT and intravasation processes (FIGS. 18, 24), at least partially mediated through FOXM1 cell signaling pathway (FIGS. 19, 20, 26). BT treatment also led to pronounced mitochondrial ultrastructural changes characterized by mitochondria swelling and disorganized cristae in PANC-1 cells (FIGS. 8, 25A, 25B). Furthermore, the gene expression of voltage gated ion channels, including Nav1.5, Kv1.3, and KCa3.1 were significantly changed in BT treated PANC-1 cells compared to that of the sham control (SC) group (FIG. 27) and the cell membrane voltage potential appears to be linked to FOXM1 gene. Similarly, the intracellular calcium was significantly affected by BT (FIG. 33). The PANC-1 tumor bearing mice treated by BT had significantly less liver metastasis compared to the SC and colony-control (CC) mice in a repeated study (FIG. 28) and the antimetastatic potential of BT was further confirmed in KPCY mouse orthotopic model.

These studies indicate a therapeutic role for BT in treatment of cancer, especially in pancreatic cancer.

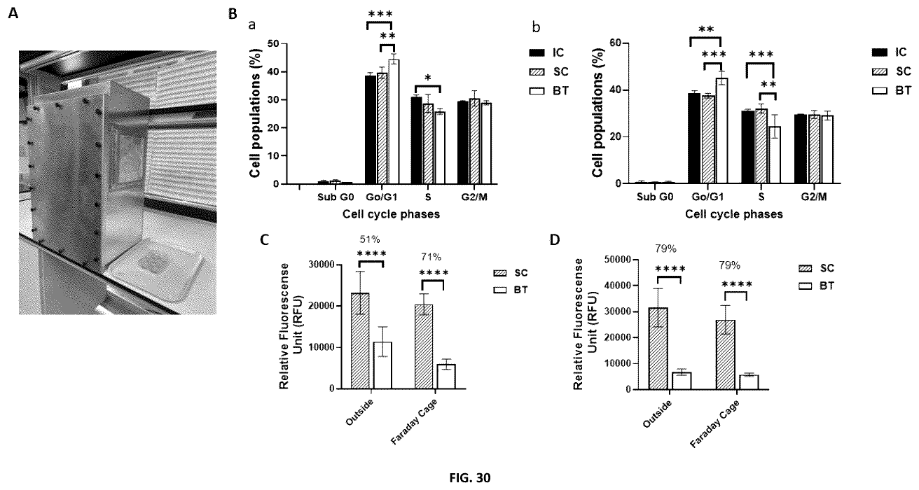

We also examined whether the EMF was a plausible mechanism of BT treatment using well accepted EMF shield device, a Faraday cage. BT induced G1 phase arrest and anti-invasiveness were similar in the cells placed outside or inside the faraday cage, suggesting EMF is not directly involved in BT treatment to the PDAC cells (FIG. 30). This indicates a non-EMF mechanism for BT's action.

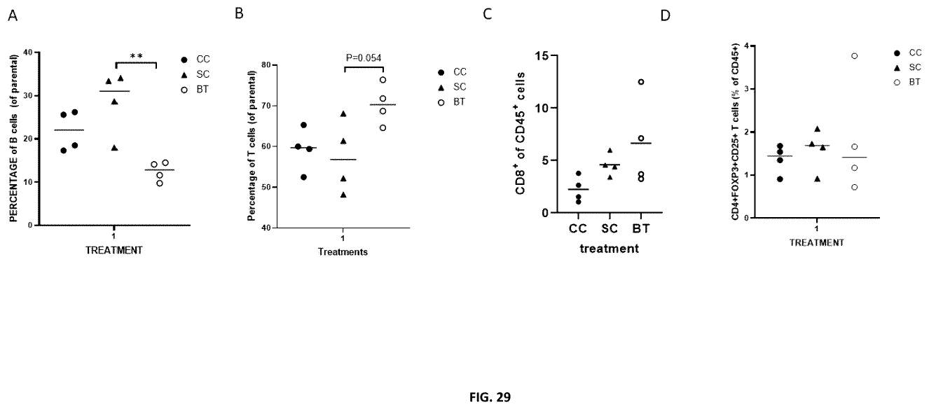

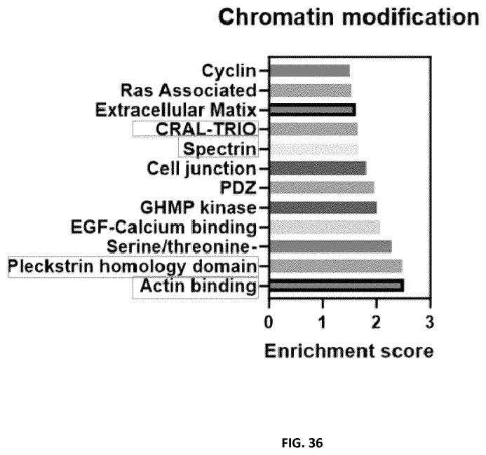

In summary, BT exerts antitumor activity, especially anti-invasion and metastasis in both in vitro pancreatic cancer cells and their relevant orthotopic models. BT led to changes in bioelectricity, cell membrane and mitochondrial membrane potential as well as cytoskeleton changes, with epigenetic data showing 7 out of top 10 cluster genes altered by BT (including Actin, plecktrin, spectrin, TRIO and PDZ) being associated with the cytoskeleton and/or intracellular communication network involving cytoskeleton (FIG. 36). In vitro data also showed that BT inhibited cell migration and invasion of PDAC cells, at least partially mediated by FOXM1 gene. In vivo data from additional animal models also demonstrated that BT has the potential to reduce the liver metastasis of the pancreatic cancer. Finally, we found that BT can alter the immune TME (FIG. 29).

### Example 1

Research was performed to evaluate the potential role of BT in anti-invasiveness in human pancreatic ductal adenocarcinoma (PDAC) cells and animal models. It was found that PANC-1 cells treated with BT (15 min) displayed 55% less invading cells than that of untreated incubator control and sham control cells (P<0.001). Similar results were observed in human PDAC L3.7 cells and mouse pancreatic cancer KPCY cells. Additionally, migration capacity was tested using the transwell migration and scratch assays. Consistent with the result of cell invasion assays, BT treatment also led to significant reduction of migration of PANC-1 and L3.7 cells by 34% and 41%, respectively, compared to sham controls.

Two additional therapists independently replicated the anti-invasiveness and anti-migratory effects of BT on these PDAC cells. This suggests the replicability of these findings across studies and therapists. Further, in PANC-1 cells exposed to BT, expression of E-cadherin was significantly upregulated, while N-cadherin, a marker of mesenchymal cells, was significantly decreased compared to the sham control group. Changes in other regulators associated with migration and invasion such as MMP2, MMP9, VEGF, and uPA were also examined, but BT treatment only had a moderate effect on these markers, indicating a slowdown in the intravasation process (i.e., EMT transition), of PANC-1 cells.

In a repeated PANC-1 mouse orthotopic model, it was found that only 28% of BT treated mice had visible liver nodules whereas liver metastasis was observed in 55% and 60% of mice in untreated colony control and sham control groups, respectively. Furthermore, BT treatment significantly reduced liver tumor burden by 73% compared to that of the sham control group (p<0.05), which was similar to that observed in the previous study. Similar to the prior PANC-1 mouse model, BT treatment had only a moderate impact on the growth of primary pancreatic tumor. Together, these findings suggest that exposure to BT reduced cellular invasion and migration processes and profoundly reduced metastasis of pancreatic tumor in an animal model.

### Biofield Therapy Suppresses Pancreatic Cancer Cell Growth and Metastases in Part Through the FOXM1 Signaling Pathway.

Pancreatic ductal adenocarcinoma (PDAC) is one of the most aggressive cancers and currently the third leading cause of cancer-related death in the United States¹. PDAC is expected to surpass colorectal cancer and to become the second leading cause of cancer-related death by 2030. Despite improvements in surgical techniques and systemic and targeted therapies, patients with PDAC still have a dismal prognosis and experience significant cytotoxicity from conventional chemoradiotherapy², with a 5-year overall survival rate of less than 13% and average survival duration from diagnosis of less than 6 months, which is the lowest of any epithelial cancer³. Pancreatic cancer is only modestly affected by most existing treatments, including immunotherapy, largely due to the presence of an excessive desmoplastic stroma, which restricts penetration of drugs, and an immune-suppressive tumor microenvironment^{4,5}. Cancer-associated mortality in PDAC is primarily caused by early metastasis and therapeutic resistance or recurrence, and earlier diagnosis and more effective treatment of PDAC are current and important unmet medical needs. Thus, tremendous efforts have been made to identify novel and effective alternative approaches with reduced cytotoxicity.

Biofield therapies (BTs) may provide useful additions to conventional treatment approaches for PDAC. The National Center for Complementary and Integrative Health classifies BTs as “energy therapies”. Preclinical studies have shown some evidence that BT modifies cellular function and tumor growth (Gronowicz G, et al., Evid Based Complement Alternat Med. 2015; 2015:926565). However, whether BT can affect the development and progression of pancreatic cancer and its potential mechanisms has not been studied.

Focus is increasing on the idea of cancer as a progressive loss of the organizational capacity of the environment, rather than the heterogeneous behavior of isolated cells (Bissell M J, et al., Nature reviews Cancer. 2001; 1(1):46-54; Ingber D E. Semin Cancer Biol. 2008; 18(5):356-364). In complement to the large body of work on chemical and biomechanical factors that mediate cancer suppression and transformation, it is now also appreciated that endogenous spatiotemporal differences in resting cell membrane potential and bioelectric gradients are an important layer of the dysregulation of cell-cell interactions that leads to cancer. Emerging data suggest that the cell membrane voltage potential (Vₘ), the voltage across the plasma membrane that arises because of the presence of different ion channels/transporters with specific ion selectivity and permeability, plays an important role in cancer cell proliferation and metastasis. See Chernet B, et al., J Clin Exp Oncol. 2013; Suppl 1; Yang M, et al., Front Physiol. 2013; 4:185; Chernet B T, Levin M. Dis Model Mech. 2013; 6(3):595-607.

We conducted a series of studies to explore the effects of BT in different in vitro and in vivo cancer models of PDAC (preregistered at https://osf.io/qtbvf/wiki/home/). In addition to replicating the experiments with cancers from other organ sites, we conducted an in-depth evaluation to determine the effects of BT on: 1) proliferation and invasion of PDAC cells, human PDAC-derived organoids, and their relevant molecular mechanisms; 2) visible cellular morphology change; 3) resting Vₘ of cells as a mechanistic link between BT and cancer phenotype; and 4) an orthotopic pancreatic cancer mouse model. We also sought to replicate key in vitro studies using three biofield therapists.

### Materials and Methods.

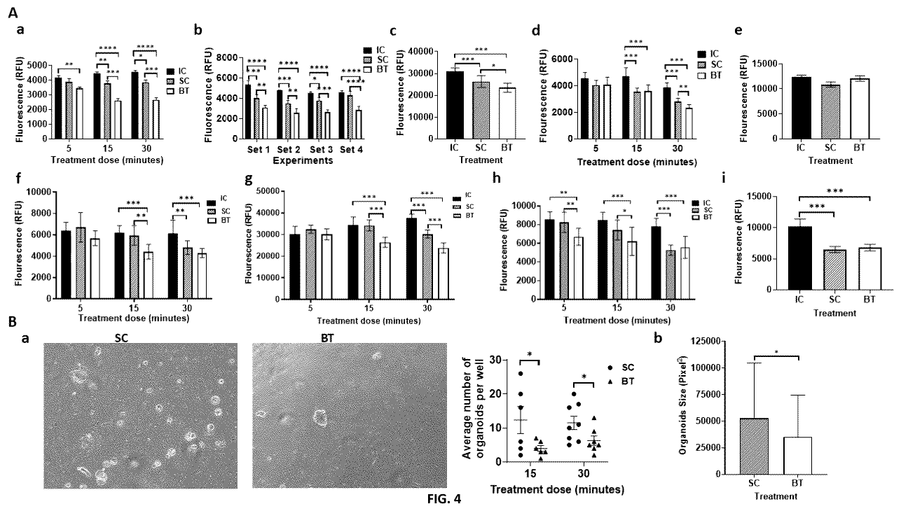

The methodologies and materials as set forth in Example 3, with additional details provided here, were used to conduct the studies and to obtain the following insights and results, as further detailed in FIGS. 4-22.

### Cell Lines and Patient-Derived Organoids (PDO).

Normal human pancreatic ductal epithelial cells HPDE-1/E6E7, and mouse pancreatic cancer Panc02 cells were used as described previously (Huang C, et al., Cancer research. 2012; 72(3):655-665). Panc02 cells were routinely cultured as described in Example 3 for PANC-1 and other cells. HPDE-1/E6E7 cells were cultured in Gibco Keratinocyte SFM (Cat #17-005-042) with supplements of bovine pituitary extract (25 mg/500 ml) and EGF 2.5 ug/500 ml (Invitrogen). All experiments were performed with mycoplasma-free cells.

Pancreatic cancer PDOs (AM67) were procured from Dr. Maitra at The University of Texas MD Anderson Cancer Center. The organoids were dislodged using 2 mg/mL dispase in phosphate-buffered saline (PBS) for 15 minutes, followed by washing with PBS at 1400 rotations per minute for 2 minutes and dissociation with Triple E for 5 minutes at 37° C. TryplE was quenched with Wash Media, cells were centrifuged again for 5 minutes at 125×g, and dissociated cells were counted and resuspended in 350-700 μL (depending on cell pellet size) of Geltrex LDEV-Free, hESC-Qualified, Reduced Growth Factor Basement Membrane Matrix (Gibco) and plated as domes on a Nunclon Delta Surface 12-well plate (ThermoFisher). Matrigel was allowed to solidify for up to 45 minutes at 37° C., at which point approximately 1 mL of PaTOM media68 supplemented with Y-27632 dihydrochloride (Tocris) was added to each well of the 12-well plate, to ensure full submergence of organoid domes. Organoids were grown for a minimum of 7 days after seeding before being passaged for the first time.

Laboratory members and the pathologist were blinded to group assignment when conducting the assays or evaluating the animals.

### Cell Proliferation and Growth In Vitro.

Human or mouse pancreatic cancer cells (1×10⁴) were seeded in 96-well plates in triplicate. After incubation for 16-24 hours, cells were exposed to BT. Immediately after the treatment, cell growth was assessed by a PrestoBlue assay (Yang P, et al. Scientific reports. 2019; 9(1):6428).

To assess the growth of PDOs, acid solubilized Rat tail collagen I (3 mg/ml, Corning, USA) was used. It was first neutralized with 375 μl of 10X DMEM (with Phenol red) and 1N NaOH (100 μl). Neutralized Collagen I (25 μl) was used to make underlays in 12 wells plate (NUNC, Life Technology) and was polymerized on ice for two hours until the underlays turned cloudy and were transferred to 37°° C. at least 30′-1 h before cell plating. About 500 single pancreatic organoid cells were mixed with cold neutralized collagen (Padmanaban V, et al., Nat Protoc. 2020; 15(8):2413-2442) and were plated gently over the underlays on a 37° C. heating plate. Plates were incubated at 37° C. in presence of 5% CO2 1 h for gelation before adding organoid culture medium. They were then treated by BT. Tumor organoid cells were continued growing in the incubator for 3-6 days and the growth of AM67 was monitored with EVOS M7000 Imaging System (Thermo Fisher Scientific) and quantified by ImageJ.

### Cell Voltage Characterization.

Briefly, cells (5-10×10³) were plated the day prior to treatment. After the treatment, plates were cultured for 5 days in the incubator at 37° C. in 5% CO₂ and 95% humidified air. DiBAC4(3), a slow-response potential-sensitive probe, was used to stain and measure the depolarization of the cells. The working DiBAC4(3) solution (1 mg/mL) was prepared by mixing the stock solution with Dulbecco modified Eagle medium-free fetal bovine serum at a ratio of 1:4000. Cells were washed with PBS and DiBAC4(3) working solution together with 1 drop of NucBlue Live Ready Probes Reagent and incubated for 15 minutes at 37° C. Green fluorescent protein and DAPI images were captured by the EVOS M5000 Imaging System (AMF5000) or EVOS M7000 Imaging System (AMF7000). Fluorescence intensity was quantified by ImageJ for the images captured by EVOS M5000 or by Cellslate software for the images captured by EVOS M7000.

### Reverse Phase Protein Array (RPPA).

Immediately after being exposed to BT for 15 or 30 minutes, PANC-1 cells were lysed in the lysis buffer provided by the Functional Proteomics Core Facility at MD Anderson. Cell lysates were then subjected to Reverse Phase Protein Array (RPPA) analysis by the Functional Proteomics Core Facility at MD Anderson as described previously (Yang P, et al. Scientific reports. 2019; 9(1):6428).

### Animal Models of Pancreatic Cancer.

Procedures: Two separate animal studies were conducted. As described in Example 2, mice with tumor size 1-10 mm² were grouped into four groups. For study #1, n=11-16 mice per group; for study #2, n=25-30 mice per group.

### Transmission Electron Microscopy.

Immediately after biofield therapy (BT), PANC-1 cells were washed with phosphate-buffered saline (without Ca⁺⁺/Mg⁺⁺) prior to treatment with EM fixative containing 3% glutaraldehyde and 2% paraformaldehyde in 0.1M cacodylate buffer (pH 7.3). The cells were then washed in 0.1M sodium cacodylate buffer and treated with 0.1% Millipore-filtered cacodylate buffered tannic acid, post-fixed with 1% buffered osmium and stained with 0.1% Millipore-filtered uranyl acetate, prior to dehydration with increasing concentrations of ethanol followed by infiltration and embedding in LX-112 medium. The samples were then polymerized in a 60° C. oven for approximately 3 days. Ultrathin sections were cut using a Leica Ultracut microtome (Leica, Deerfield, IL) and stained with uranyl acetate and lead citrate in a Leica EM Stainer. The samples were examined in a JEM 1010 transmission electron microscope (JEOL USA, Inc., Peabody, MA) using an accelerating voltage of 80 kV. Digital images were obtained using an AMT imaging system (Advanced Microscopy Techniques Corp, Danvers, MA).

For the scratch assay, the Culture-Insert 2 Well in μ-Dish 35 mm (Ibidi, Fitchburg, WI) was used. The dish has culture-inserts to create a reproducible gap size. MiaPaca-2 cells (6×10⁵) were seeded and cultured in each of the two culture-insert wells for 24 hours. The insert was then gently removed by sterile tweezers and an aliquot of 1 mL of culture medium was added to the culture dishes. The cell culture dishes were placed in two separate CytoSmart FL microscopes (Axion Biosystem, Atlanta, GA) inside two incubators, and the cell culture dishes were exposed to sham control or BT. Images were captured every 30 minutes for 48 hours. The images were analyzed by CytoSmart scratch assay online program.

### Cell Cycle Analysis.

For cell cycle analysis, cells (2.5×10⁵) cultured in 6-well plates were exposed to BT or sham control for 15 minutes. Cells were immediately trypsinized and centrifuged. The cell pellets were suspended, washed with 1X PBS, and fixed in 70% ethanol at 4° C. overnight. The fixed cells were further washed with 1X PBS and suspended in PBTB staining solution containing PBS, propidium iodide (5 μg/mL), and DNase-free Rnase A (10 μg/mL). Cells were incubated in the dark at room temperature for 15 minutes and put on ice until data were acquired using a BD FACS Caliber flow cytometer (BD Biosciences, San Jose, CA). The percentage of cells in each phase of the cell cycle was determined from the DNA histogram content.

### Immunoblotting.

Immunoblotting was performed using an automated Western blotting system: the Jess Simple Western system (ProteinSimple, San Jose CA, USA) automatically separates and immunodetects proteins by size through its capillaries. To quantify the protein expression of interest in the treated cells, we followed the standard method for the 12-230 kDa Jess separation module (SM-W004). The final protein concentration of tissue lysate (0.4 to 1 mg/mL) was obtained by mixing measured cell lysate (0.6 to 2 mg/mL) with 0.1X sample buffer and Fluorescent 5X Master mix (ProteinSimple). This mixture was denatured at 95° C. for 5 minutes. Primary antibodies of interest are indicated in the Results section, and β-actin or tubulin were prepared by mixing antibody diluent at a ratio of 1:10 to 1:50. Secondary antibodies were prepared by combining chemiluminescence antibody and fluorescence antibody. After that, cell lysate samples were loaded into the Ladder plate (12-230 kDa), followed by loading of antibody diluent, primary antibody, secondary antibody, a luminol-s and peroxide mixture (for chemiluminescence detection), and wash buffer according to the manufacturer instructions. Digital images of chemiluminescence and fluorescence for the proteins of interest were captured with Compass Simple Western software (version 4.1.0, Protein Simple), and the area was used for quantification of the proteins.

### Results.

### BT Decreased Cell Growth of Human and Mouse Pancreatic Cancer Cells and PDOs in Cell Culture.

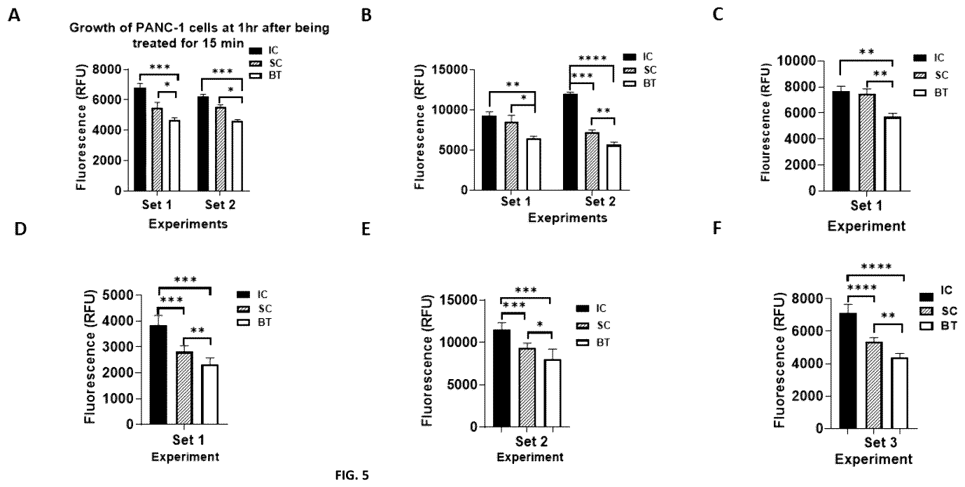

We first examined the proliferation of human (PANC-1 and MiaPaCa-2) and mouse (Panc02 and KPCY) pancreatic cancer cells using PrestoBlue assay after 5, 15, or 30 minutes of BT treatment. As illustrated in FIG. 4Aa, PANC-1 cells exposed to BT for 15 minutes and 30 minutes had moderately but significantly reduced cell growth at 1 hour after BT compared with the IC and SC groups. Cell growth was also reduced in the SC group (i.e., the cells placed on the counter as the BT group), but the reduction was greater for BT compared with both other groups. Given that 5-minute BT did not lead to significant changes in PANC-1 cell growth and the effects of 15-minute and 30-minute BT on the growth of PANC-1 cells were similar, we repeated the experiments using the 15-minute dose in more than 12 different experiments (4 replicates shown in FIG. 4Ab; 5 replicates shown in FIG. 5A-C; all experiments conducted on separate days). Similar activity was also observed in human pancreatic cancer COLO357 cells (FIG. 4Ac) and MiaPaCa-2 cells (FIG. 6A), as well as in mouse pancreatic cancer Panc02 cells, after exposure to BT. In contrast to PANC-1 cells, BT at the 30-minute dose seemed to show the greatest decrease in cell growth relative to that of the IC and SC groups in Panc02 cells (FIG. 4Ad). We then treated the Panc02 cells with BT for 30 minutes in three separate experiments and observed that BT consistently and significantly inhibited the growth of Panc02 cells compared with the IC and SC groups (FIG. 5D-F). BT also significantly reduced the growth of KPCY (mouse pancreatic cancer) cells after 15-minute exposure (FIG. 6B). To further understand whether the suppressive effect of BT on cell growth was specific to cancer cells, we exposed immortalized human pancreatic ductal epithelial cells (HDPE-1/E6E7) to BT for 15 minutes. As shown in FIG. 4Ae, HPDE-1/E6E7 cells exposed to BT had a similar growth rate to that of IC group, but slightly higher than that of the SC group, suggesting that BT has a potential to reduce the growth capacity of PDAC cells in vitro.

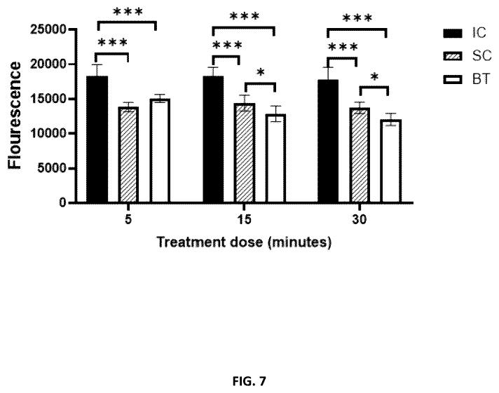

The experiments using PANC-1, COLO357, and L3.7 cells were repeated with a second biofield therapist (Therapist 2). As shown in FIG. 4Af-h, 15-minute BT led to consistently and significantly slower growth in all three cell lines relative to both IC and SC. Although the SC HPDE-1/E6E7 cells had a significantly lower growth rate than that of the IC HPDE-1/E6E7 cells, the growth rate in the BT group was similar to that of the SC group (FIG. 4Ai), which was consistent with the results observed in HPDE-1/E6E7 cells treated by the first biofield therapist. The PANC-1 cells treated by Therapist 3 for both 15 and 30 minutes had moderate, but significantly lower growth rates than those observed in the IC and SC groups (FIG. 7). Thus, the results from human PDAC cells treated by three different biofield therapists consistently showed that BT reduced cell growth in human PDAC cells. Given that PDOs represent the genetic and epigenetic landscape of each PDAC patient and are a reliable model to understand cancer biology and predict treatment, we further tested the growth of AM67 PDOs after BT. The number of PDOs in the BT group was significantly less than that of the SC group 11 days after BT (FIG. 4Ba). Similarly, BT also led to reduction in the size of the AM67 PDOs compared to that of the SC group (FIG. 4Bb), further supporting the potential role of BT in suppressing PDAC growth.

### BT Altered the Ultrastructure of PANC-1 Cells.

Cell organelles exist in highly dynamic cellular networks that communicate with each other to carry out fundamental functions. The ultrastructural changes in cellular organelles were examined immediately after BT treatment of PANC-1 cells using transmission electron microscopy (TEM). BT treatment led to pronounced mitochondrial ultrastructural changes. As shown in FIG. 8, PANC-1 cells in the IC (FIGS. 8A and 8D) and SC (FIGS. 8B and 8E) groups had elongated mitochondria with well-organized cristae. In contrast, mitochondrial cristae were disordered and mitochondria were markedly dilated and swollen in PANC-1 cells exposed to BT (FIGS. 8C and 8F), This profound and non-subjective morphology change in mitochondrial ultrastructure not only substantiates but also provides new insight into the biological action of BT on important cellular function.

### BT Blocked the Cell Cycle in the G0/G1 Phases in Human PDAC Cells.

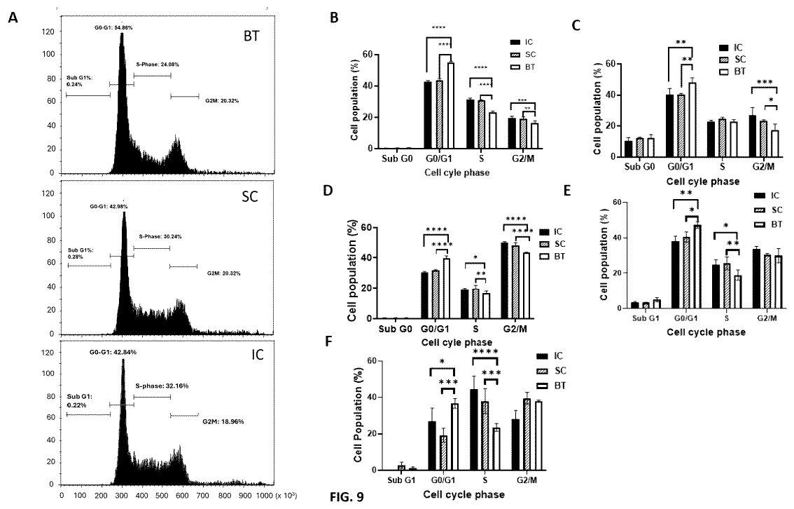

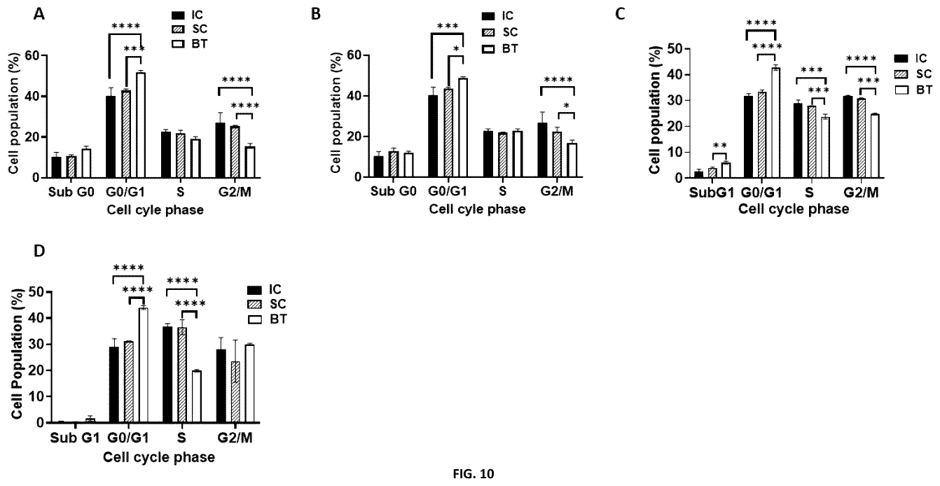

To further understand the biological changes of pancreatic cancer cells after BT, we performed cell cycle analysis of human PDAC cells. As shown in FIG. 9, BT led to significant 1.5-fold increases in G0/G1 phase cells in PANC-1 cells compared with the IC and SC groups (FIGS. 9A and 9B). We then repeated the study three more times and found the same changes in the cell cycle pattern occurring in all experiments (FIG. 9C and FIGS. 10A and 10B). To confirm that the cell cycle changes observed in PANC-1 cells after BT were independent of baseline cell growth, we then plated PANC-1 cells 2 days prior to treatment, treated them with the BT, and collected the cells immediately after treatment. As shown in FIG. 10D, even though the G0/G1 phase population of cells was lower and the G2/M phase population of cells almost double in the IC and SC groups compared with those shown in FIGS. 9B and 9C, PANC-1 cells exposed to BT were arrested in the G0/G1 cell cycle phase. Similarly, BT led to significant G0/G1 phase arrest in other human PDAC cells, including L3.7 cells (FIG. 9E) and COLO357 cells (FIG. 9F). Furthermore, PANC-1 cells treated by the other two biofield therapists for 15 minutes also displayed significantly higher G0/G1 phase cell populations than those of the SC or IC groups (FIGS. 10C and 10D).

### BT Reduced Pancreatic Cancer Cell Membrane Potential, Leading to Hyperpolarization in Multiple Pdac Cells.

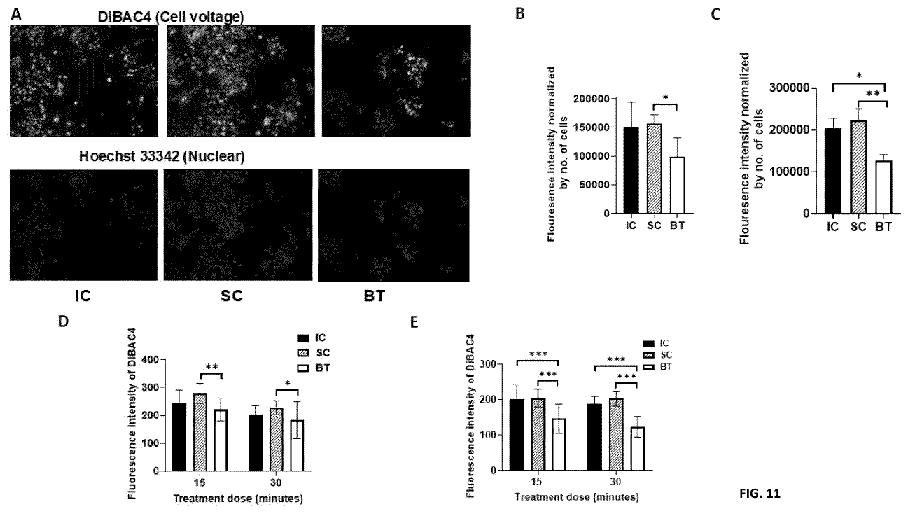

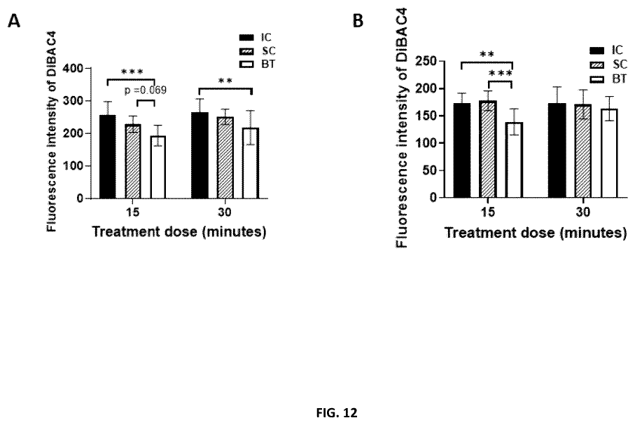

Given the known roles of Vₘ in maintaining and suppressing the cancer phenotype (Chernet B, Levin M. J Clin Exp Oncol. 2013; Suppl 1; Chernet B T, Levin M. Oncotarget. 2014; 5(10):3287-3306), we examined whether resting Vₘ could be part of the mechanism by which BT exerts its effects. As shown in FIG. 11, 5 days after exposure to BT, SC, or IC, IC and SC PANC-1 cells had similar fluorescence intensity of DiBAC4, whereas PANC-1 cells exposed to BT had significantly weaker staining relative to the other two groups (FIGS. 11A and 11B). When PANC-1 cells were exposed to BT for two consecutive days, the Vₘ was reduced by almost 50% compared with that of the other two groups (p<0.05 and p<0.01, respectively; FIG. 11C). The effects of BT on decreasing Vₘ were also detected in MiaPaCa-2 cells (FIG. 11D, FIG. 12A) and KPCY cells (FIG. 11E, FIG. 12B) immediately or 1 hour after BT (15 or 30 minutes). Together, these findings suggest that BT can alter Vₘ, which reflects a hyperpolarization of pancreatic cancer cells that could contribute to the slower growth of the cells.

### BT Inhibited Migration and Invasion in PDAC Cells.

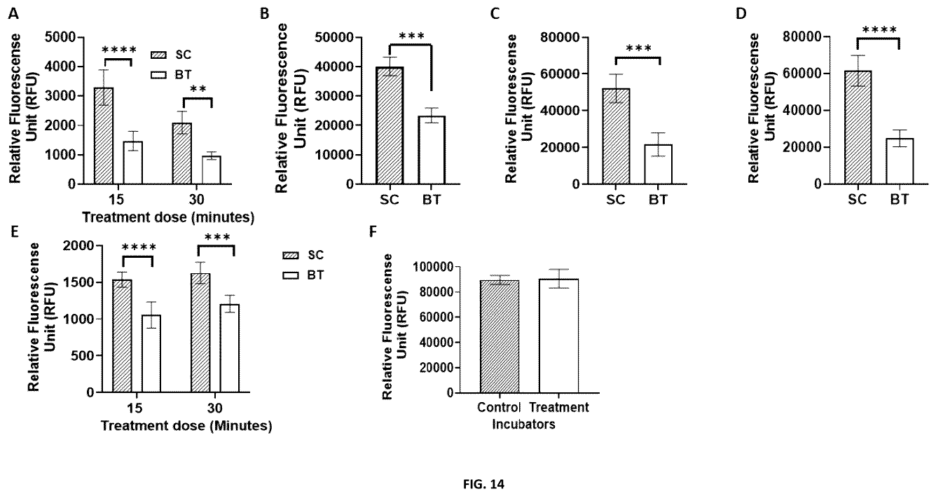

The effect of BT on the invasiveness of PDAC cells was examined. As shown in FIG. 13Aa, cells exposed to BT displayed 55% less invasiveness compared with IC and SC cells (p<0.0001), with no differences between the IC and SC groups. Given that there were no differences between IC and SC groups, we repeated the experiment four additional times on different days with the BT and SC groups, and the results consistently showed that BT led to significant suppression of the invasiveness of PANC-1 cells (FIG. 14A-D). Anti-invasiveness was further examined in human PDAC cells with relatively lower expression of FOXM1 (COLO357) and higher expression of FOXM1 (L3.7, a metastatic clone of COLO357 cells) (Huang C, et al., Clinical cancer research. 2014; 20(6):1477-1488), as well as in mouse pancreatic tumor KPCY cells. L3.7 cells exposed to BT exhibited a similar reduction in invasiveness to that observed in PANC-1 cells (FIG. 13Ab and FIG. 14E), whereas COLO357 cells exposed to BT displayed less invasiveness (22%) than that seen in PANC-1 cells (FIG. 13Ac). BT also reduced the invasiveness of KPCY cells to a similar degree to that observed in PANC-1 cells (FIG. 13Ad).

Consistent with the results of the cell invasion assays, BT also led to significant reduction of migration in PANC-1 and L3.7 cells, by 34% and 41%, respectively, compared with SC (p<0.001; FIGS. 13Ae and 13Af). The inhibitory effect of BT on migration in PDAC cells was confirmed in MiaPaca-2 cells by the scratch migration assay (FIG. 13B). The anti-invasiveness and anti-migrative effects of BT in these PDAC cells were tested with Therapist 2, and the results were similar to those observed with the first therapist (FIG. 13C), indicating replicability of these findings across studies and therapists.

To confirm that the BT-elicited anti-invasiveness in the PANC-1 cells was not caused by the difference in incubators used, the SC and BT cells were incubated in after the treatment, we examined the invasiveness of PANC-1 cells without treatment when they were incubated in the SC and BT incubators. As shown in FIG. 14F, the invasive capacity of PANC-1 cells in both incubators was similar.

### BT Modified Key Cell Signaling Proteins, Especially FOXM1.

RPPA analysis in PANC-1 cells was conducted to further understand the potential mechanisms involved in BT-elicited effects compared with the changes observed in the SC group. This was done in a blinded fashion, with Core Facility members unaware of the experimental design or groups.

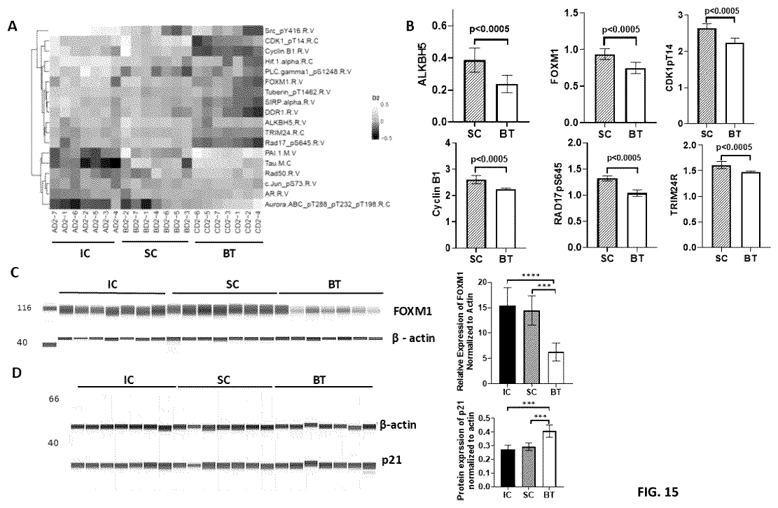

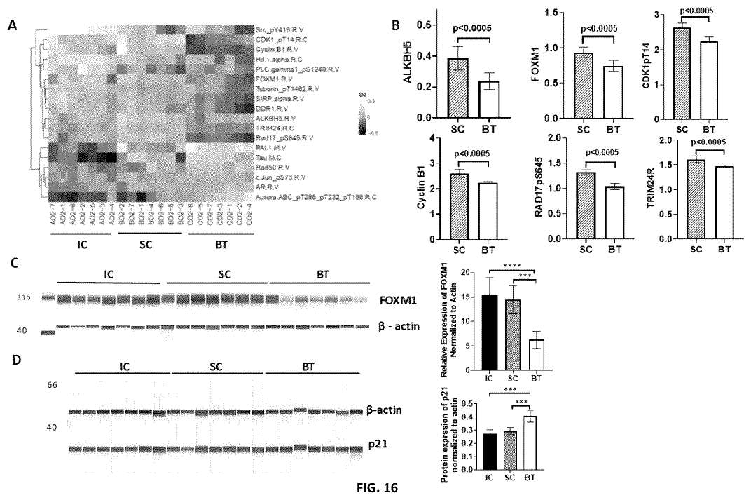

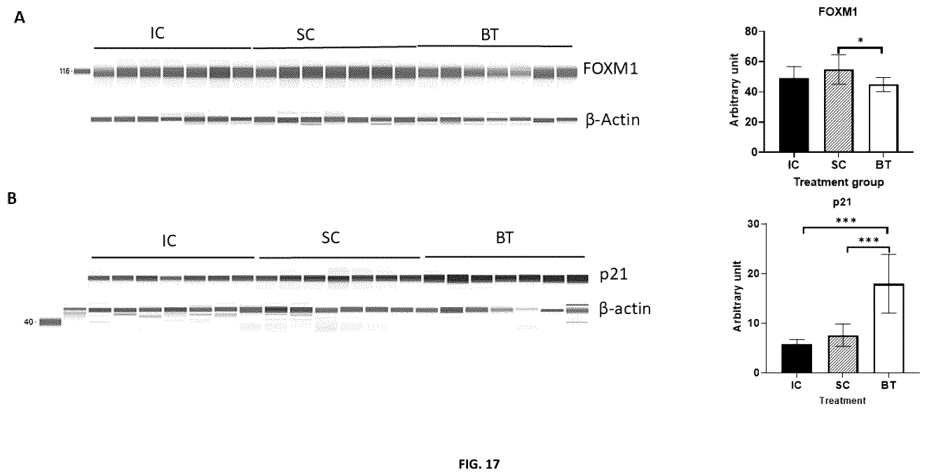

Expression of several proteins was altered by BT administered for both 15 and 30 minutes, as shown in FIGS. 15A and 15B. Although some proteins were upregulated by BT, 15-minute BT significantly downregulated ALKBH5 (a known m6 demethylase and epitranscriptional regulator for FOXM1) by 41%. FOXM1 was downregulated by 21%, and cyclin B1 and pCDK1 were each downregulated by 15% (p<0.0005 for all proteins; FIG. 15B). Similar reductions in the expression of cyclin B1 and FOXM1 were observed after 30-minute BT in PANC-1 cells, along with reductions in TRIM24, TFRC, and PAI-1M (FIG. 16). The significant downregulation of FOXM1 protein by BT (15-minute) in PANC-1 cells was further confirmed by automated Western Blot analysis (Jess; FIG. 15C). Because p21 arrested the cells in both the G1 and G2/M phases, we examined whether BT also affects p21 proteins. As shown in FIG. 15D, BT indeed upregulated p21 expression in PANC-1 cells. Similar results were observed in L3.7 cells exposed to BT (FIG. 17). These findings suggest that BT can potentially affect the cell cycle of PANC-1 cells by modulating transcription regulators of the cell cycle or cyclin kinases such as FOXM1 and p21.

### BT Altered Epithelial-Mesenchymal Transition-Associated Markers in PANC-1 Cells.

To understand how BT suppressed the invasiveness of the PDAC cells, we examined expression markers associated with epithelial-mesenchymal transition and invasion in PANC-1 cells. As shown in FIG. 18, the expression of E-cadherin, a marker of epithelial cells, in PANC-1 cells exposed to BT was significantly higher than in the IC and SC groups (FIG. 18A). In contrast, N-cadherin, a marker of mesenchymal cells, was significantly lower in the BT group than in the IC and SC groups (p<0.05; FIG. 8B).

BT also significantly reduced CD44 protein expression compared with IC (FIG. 18C), suggesting that BT slowed down the intravasation process, i.e., epithelial-mesenchymal transition of PANC-1 cells. CD44 is one of the important PDAC stemness markers.

Knockdown or overexpression of CD44 affects the PDAC stemness as indicated by spheroid formation assay which is a well recognized the method to demonstrate the stemness of the cancer cells (Yan Y, et al. Cancer Res. 2016 Apr. 15; 76(8):2419-31). BT treatment significantly down-regulated the protein expression of CD44 in PANC-1 cells, suggesting BT could potentially inhibit the stemness of the PDAC cells.

### Modification of the FOXM1 Gene Altered the Response of PANC-1 Cells to BT.

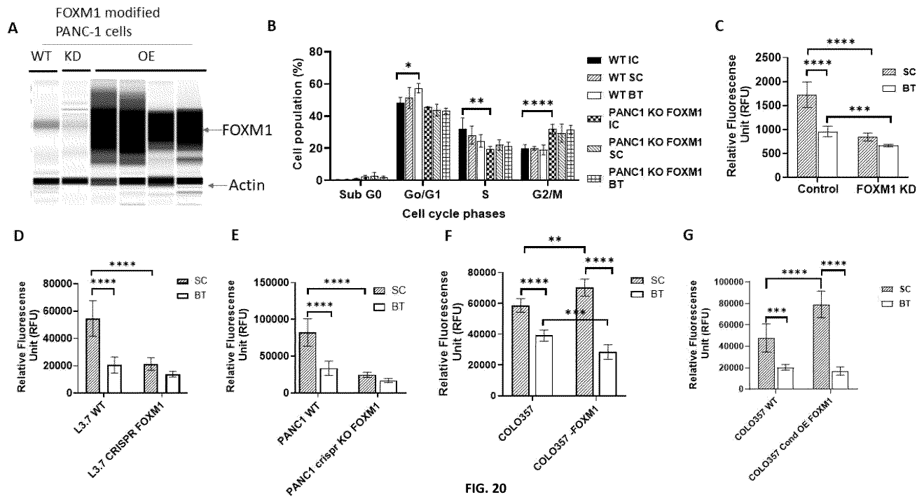

To determine whether FOXM1 is a potential molecular target responsible for BT-elicited changes, we first examined the correlation between the ability of BT to downregulate FOXM1 protein expression and the suppressive effect of BT on the invasion of PDAC cells. As shown in FIG. 19, the correlation was high (R²=0.85) suggesting that the stronger the suppressive effect of BT on the expression of FOXM1 the greater the inhibition of BT on cell invasion. BT had the greatest effect in suppressing FOXM1 in PANC-1 cells (>40%) with the highest inhibition of invasion (54%) and BT did not suppress FOXM1 in COLO357 cell which had the least inhibition of the cell invasion (17.3%). This suggests that BT-induced anti-invasiveness in PDAC cells might be at least in part mediated by its ability to down regulate FOXM1 protein. We then examined the cell cycle and invasiveness of parental PANC-1 cells and FOXM1-modified PANC-1 cells after BT or SC. In the FOXM1 stable-knockdown PANC-1 cells, the expression of FOXM1 was reduced by almost 90% compared with that of parental PANC-1 cells (FIG. 20A). When the parental PANC-1 and FOXM1 stable-knockdown PANC-1 cells were exposed to BT, the parental PANC-1 cells showed a higher population of G0/G1-phase cells compared with SC, as did the FOXM1 stable-knockdown PANC-1 cells (FIG. 20B). When the parental PANC-1 cells were exposed to BT (15 minutes), the invasiveness of these cells was significantly reduced relative to SC (p<0.001; FIG. 20C), and relative to previous experiments (FIG. 13Aa and FIG. 14A-D). FOXM1 knockdown reduced the invasiveness of the PANC-1 cells by almost 50%, which is similar to the effects of BT, and there were no differences in invasiveness between the BT and SC groups (FIG. 20C). Although BT led to a significant 62.2% reduction of invasiveness in non-modified L3.7 cells relative to SC (FIG. 20D and FIG. 13Ab, FIG. 14E), knocking out FOXM1 in L3.7 cells significantly reduced invasiveness by 61% compared with parental L3.7 cells, and there were no longer differences between the BT and SC groups (FIG. 20D). Similarly, FOXM1 Knock out (KO) PANC-1 cells displayed a significant lower number of invaded cells compared to that of parental PANC-1 cells, and again BT-induced suppression of PANC-1 cell invasiveness was minimized after knocking out of FOXM1 (20E), which was consistent with the FOXM1 KO L3.7 cell outcome (FIG. 20D). Conversely, overexpressing FOXM1 in COLO357 cells significantly increased the number of cells invaded by 19% compared with that of parental COLO357 cells. Additionally, BT led to a significant 33.6% reduction of cell invasiveness in parental COLO357 cells, whereas FOXM1-overexpressing COLO357 cells showed significantly reduced invasiveness, by about 59.5% compared with the SC group (FIG. 20F). Additionally, we investigated the anti-invasiveness of BT in COLO357 and PANC-1 cells with conditional over-expression of FOXM1 gene using Tet-on Tetracycline (or Doxycycline) inducible system. Again, conditional overexpressing FOXM1 in COLO357 cells significantly increased the number of cells invaded by 65.6% compared with that of parental COLO357 cells. BT led to a significant 57.4% reduction of cell invasiveness in parental COLO357 cells, whereas FOXM1-overexpressing COLO357 cells showed significantly reduced invasiveness, by about 78.4% compared with the SC group (p<0.0001) (FIG. 20G). These data suggest that FOXM1 might be at least partially responsible for the activity of BT as it relates to changes in the cell cycle and cell invasiveness in PDAC cells.

### BT Inhibited Liver Metastasis of PANC-1 Cells in an Orthotopic Mouse Model.

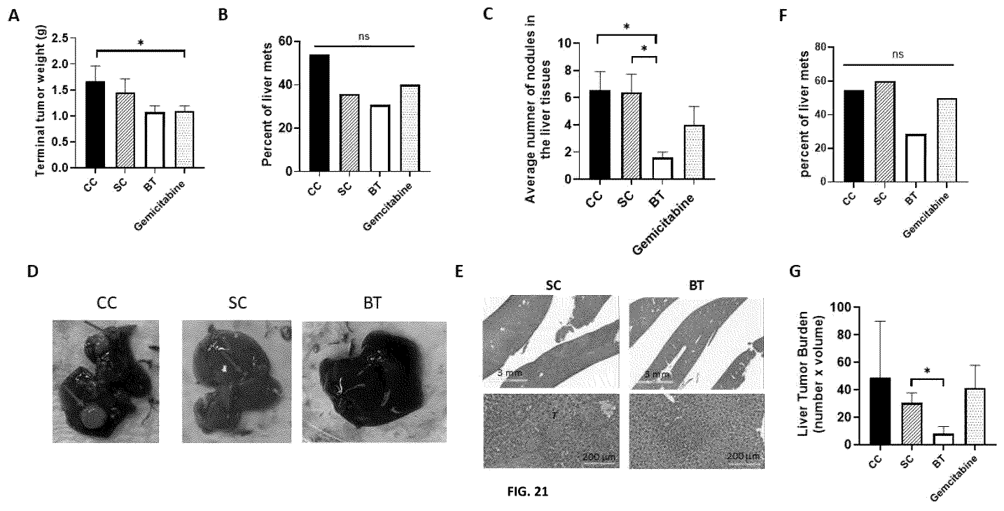

To further evaluate the antitumor effect of BT on pancreatic cancer, we conducted an antitumor efficacy study in a human PANC-1 mouse orthotopic model. As shown in FIG. 21A, the average terminal tumor weight of mice exposed to BT was 36% and 26% less than that of the CC and SC groups, respectively. However, the difference did not reach statistical significance. Gemcitabine (one-quarter maximum tolerated dose, 40 mg/kg) led to a significant 35% reduction of the growth of PANC-1 orthotopic tumors relative to the CC group (p<0.05), with no differences between the gemcitabine and BT groups (FIG. 21A). We also observed that 30% of the mice in the BT group had liver metastasis, which was 43% less than in CC group (53.8% had liver metastasis) and 17% less than in the SC group (FIG. 21B). When the number of liver nodules in the mice with liver metastasis was examined macroscopically, the average number of liver nodules of mice exposed to BT was 1.6±0.9 per liver, which was 75% less than that of the CC group (6.6±3.6) and SC group (6.4±3.0; p<0.05). Mice treated with gemcitabine also had 39% fewer liver nodules compared with the CC group, but the difference did not reach statistical significance (FIG. 21C). Although there were more and larger liver nodules in the CC mice and SC mice, a normal-looking liver was present in the BT mice (FIG. 21D).

Histopathologic examination (FIG. 21E) of the liver nodules showed that the average number of liver nodules in the CC group was 16.3, compared with 8.7 in the BT group, which was also almost 50% less than in the SC group. When the study was repeated with the same therapist and a similar PANC-1 mouse orthotopic model, 28% of BT mice had visible liver nodules, whereas liver metastasis was observed in 55% of CC mice and 60% of SC mice (FIG. 21F). Furthermore, BT significantly reduced liver tumor burden by 73% compared with SC (p<0.05; FIG. 8G), which was similar to our observations in the first study.

### Discussion.

In the current study, we provided several lines of evidence that BT has antitumor effects both in cell culture and in human pancreatic cancer PANC-1 orthotopic mouse models of pancreatic cancer. BT consistently inhibited the growth and invasiveness of various human and mouse PDAC cells and PDOs in vitro, which was accompanied by pronounced alteration of mitochondrial morphology, significant induction of cell cycle arrest, and reduction of epithelial-mesenchymal transition marker expression. These findings were replicated across multiple experiments using stringent experimental controls, including incubator/colony controls and sham controls, and included three different biofield therapists. BT also significantly reduced the cell membrane voltage in PANC-1 cells. BT moderately inhibited primary tumor growth, but significantly reduced liver metastasis of PANC-1 cells in an orthotopic mouse model in two separate studies, suggesting that BT can slow the growth of PANC-1 tumors, and, more importantly, may be efficacious against metastatic growth to the liver. The findings from the animal models were consistent with the in vitro findings, both indicating that the magnitude of the effect of BT was stronger in terms of the anti-invasiveness (see FIG. 15) than for slowing the growth of PDAC cells (see FIG. 4).

The current study used multiple experiments to explore the cellular mechanisms of BT against PDAC, and our findings showed that BT significantly reduced FOXM1 and its signaling loop-related protein expression in PANC-1 cells. Upregulation, knockdown, and knockout of FOXM1 expression significantly modified the impact of BT on the invasiveness of PDAC cells. Taken together, these in vitro and in vivo data showed for the first time that BT is capable of modifying metabolic activity, growth, and invasiveness of PDAC cells through downregulation of FOXM1, an important regulator of the cell cycle, metastasis, and stemness of pancreatic cancer, and BT could therefore be an important therapy to explore for patients with pancreatic cancer. BT does not affect the growth of normal cells, which suggests limited toxicity and good safety profile of BT.

PDAC is known to harbor a plethora of genetic and epigenetic alterations, including activation of a KRas oncogene mutation and inactivation of CDKN2A, TP53, and SMAD4 tumor suppressors (Murphy S J, et al., Gastroenterology. 2013; 145(5):1098-1109.e1091; Su G H, Kern S E. Current opinion in gastroenterology. 2000; 16(5):419-425). Thus far, targeting these alterations for therapeutic intervention has not been effective. Alternatively, researchers are identifying and targeting downstream key effectors of these oncogenic signaling pathways. The FOXM1 transcription factor has been identified as such an effector, because multiple key oncogenic pathway signals converge to activate the expression and function of FOXM1 (Huang C, et al. Biochimica et biophysica acta. 2014; 1845(2):104-116; Li L, et al., Gastroenterology. 2014; 147(2):485-497.e418; Pandit B, et al., Cell cycle (Georgetown, Tex). 2009; 8(20):3425-3427; Park H J, et al., The EMBO journal. 2009; 28(19):2908-2918; Ma R Y, et al., Journal of cell science. 2005; 118(Pt 4):795-806), a master regulator of cell cycle progression. FOXM1 protein levels also dynamically change with cell cycle progression, peaking at the S and G2/M phases (Wang X, et al., Proceedings of the National Academy of Sciences of the United States of America. 2002; 99(26):16881-16886; Wang I C, et al., Molecular and cellular biology. 2005; 25(24):10875-10894), whereas the functional activation, nuclear translocation, and stability of FOXM1 protein is subject to its protein phosphorylation, regulated by cyclin/CDK and KRAS-MAPK/AKT signaling (Ma R Y, et al., Journal of cell science. 2005; 118 (Pt 4):795-806; Myatt S S, et al., Nature reviews Cancer. 2007; 7(11):847-859; Anders L, et al., Cancer cell. 2011; 20(5):620-634). FOXM1 is a transcription factor that is overexpressed in many human cancers, including pancreatic cancer, with functions involved in promoting cell proliferation and facilitating tumor cell invasion, metastasis, and stemness (Huang C, et al., Cancer research. 2012; 72(3):655-665; Bella L, et al., Semin Cancer Biol. 2014; 29:32-39; Quan M, et al., Mol Cancer. 2013; 12:159; Kalathil D, et al., Front Oncol. 2020; 10:626836). Elevated expression of FOXM1 protein is correlated with various human malignancies, including pancreatic cancer, and FOXM1 was shown to be a major predictor of adverse outcomes among 39 human tumor types (Huang C, et al., Cancer research. 2012; 72(3):655-665; Huang C, et al., Clinical cancer research 2014; 20(6):1477-1488; Huang C, et al., Biochimica et biophysica acta. 2014; 1845(2):104-116; Gentles A J, et al., Nature medicine. 2015; 21(8):938-945; Kong X, et al., Cancer research. 2013; 73(13):3987-3996; Pilarsky C, et al., Neoplasia (New York, NY). 2004; 6(6):744-750; Wei G, et al., Aging. 2022; 14(22):9128-9148). Altogether, these data establish FOXM1 as a promising target for cancer treatment (Borhani S, et al., Expert opinion on therapeutic targets. 2020; 24(3):205-217) and FOXM1 inhibitors are being developed for clinical application (Gormally M V, et al., Nature communications. 2014; 5:5165; Shukla S, et al., Mol Cancer Ther. 2019; 18(7):1217-1229).

The current study is the first to show that BT can significantly affect protein expression of FOXM1 and its related signaling loop genes, including ALKBH5, cyclin B1, and CDK1, in PANC-1 cells, in a blinded RPPA analysis. Knockdown of FOXM1 expression had similar effects to those observed with BT on the G0/G1 phase of cells and on cell invasion, whereas overexpression of FOXM1 attenuated the activity of BT, which substantiates the critical role of FOXM1 in mediating the antitumor activity of BT. Detailed mechanisms underlying how BT regulates FOXM1 expression and function remain to be defined. FOXM1 expression and activity is tightly regulated and coordinated by transcriptional factors (activation) and epigenetic modifications, including transcriptional and translational regulation. Human AlkB homolog H5 (ALKBH5) is an important m6A demethylase that plays an important role in various biological processes, including proliferation, invasion, and metastasis, during the development of various neoplastic conditions. ALKBH5 has been reported to demethylate nascent FOXM1 transcripts and enhance FOXM1 expression in glioblastoma. Given that BT administered for only a short time (15-30 minutes) can result in significant downregulation of FOXM1 protein expression, it is highly possible that post-transcriptional modifications at the FOXM1 mRNA and/or protein level, such as ALKBH5-mediated m6A methylation of FOXM1 mRNA or pAKT-mediated phosphorylation of FOXM1 protein, may contribute to the quick change in FOXM1protein expression after BT (Chesnokov M S, et al., Front Oncol. 2021; 11:696532; Zhang S, et al., Cancer cell. 2017; 31(4):591-606 e596). Emerging evidence suggests that FOXM1 may reciprocally promote AKT activation and establish a positive feedback loop (Borhani S, et al., Expert opinion on therapeutic targets. 2020; 24(3):205-217; Chesnokov M S, et al., Front Oncol. 2021; 11:696532). Our previous study showed the pAKT can be downregulated by BT treatment (Yang P, et al., Integr Cancer Ther. 2019; 18:1534735419840797). Thus, BT might potentially affect the network of the FOXM1 and AKT pathway in PDAC cells.

It is well appreciated that cancer cells possess distinct bioelectrical properties. Abnormal depolarization of resting membrane Vₘ has been considered as a convenient marker for neoplasia and activates a metastatic phenotype in genetically normal cells in vivo (Chernet B T, et al., Dis Model Mech. 2013; 6(3):595-607; Levin M. et al., Mol Biol Cell. 2014; 25(24):3835-3850; Fraser S P, et al., Clinical cancer research. 2005; 11(15):5381-5389; Prevarskaya N, et al., Physiol Rev. 2018; 98(2):559-621). Vₘ change triggers metastatic behaviors at a considerable distance, mediated by transcriptional and epigenetic effects of electrically modulated flows of serotonin and butyrate (Chernet B T, Levin M. et al., Dis Model Mech. 2013; 6(3):595-607). Cell membrane voltage potential is mediated through different concentrations of ions and ion channels that are now recognized to play an important role in oncogenesis (Prevarskaya N, et al., Physiol Rev. 2018; 98(2):559-621). Electrophysiologic analyses in many cancer cell types have revealed a depolarized Vₘ that favors cell proliferation and migration. Hyperpolarizing CHO cells induce mitotic arrest and cell division, and hyperpolarization inhibits oncogene-induced tumorigenesis (Chernet B, Levin M. et al., J Clin Exp Oncol. 2013; Suppl 1; one; CD, Jr., J Theor Biol. 1971; 30(1):151-181). Using a well-characterized molecule, DiBAC4, for staining the cell membrane potential, we observed that BT significantly reduced the cell membrane potential in PANC-1 and MiaPaCa-2 (human) and KPCY (murine) cells. It is well established that cell migration is regulated by water and ions and Vₘ is considered an indirect factor that can affect cell migration by regulating the electrical driving force for Ca²+ via the TRP channel (Schwab A, et al., Physiol Rev. 2012; 92(4):1865-1913; Lobikin M, et al., Phys Biol. 2012; 9(6):065002). Several members of the TRP channel, such as TRPM8, are critical in the proliferation and metastasis of pancreatic cancer cells, and expression of TRPM8 is associated with poor overall survival of patients with PDAC (Yee N S, et al., Cells. 2014; 3(2):500-516; Du J D, et al., Med Sci Monit. 2018; 24:3720-3725; Liu J, et al., Pancreatology. 2018; 18(8):935-944; Mesquita G, et al., Cells. 2021; 10(5)). Thus, how BT led to the reduction of cell Vₘ, resulting in hyperpolarization of PDAC cells and contributing to the antimetastatic activity of PDAC cells, needs to be further evaluated as well as a better understanding of cell polarization and oncogenic processes.

The physical mechanisms by which BT affects cellular processes remain unknown. Plausible mechanisms include biophoton emissions and EMFs (Roll W G, et al., Int J Neurosci. 2002; 112(2):197-224; Persinger M A, et al., Int J Yoga. 2012; 5(2):140-145; Karbowski L M, et al., Neurosci Lett. 2012; 523(2):131-134; Buckner C A, et al., PLoS One. 2015; 10(4):e0124136; Hu J H, et al., Int J Radiat Biol. 2010; 86(2):79-88). The human body is well known to emit EMFs, which are intricately involved in maintaining homeostasis overall and in many specific organ systems. Electrocardiograms and electroencephalograms are used to measure the EMFs emitted by the heart and brain, respectively, and other technologies can be used to measure human EMFs, such as examination of the emission of biophotons from the body⁶⁴. EMFs can also be measured in any living organism (Zhou S A, Uesaka M. Int J Eng Sci. 2006; 44(1-2):67-92). EMFs have been shown to modulate tumor growth, angiogenesis, and tumor necrosis factor in animals (Crocetti S, et al., PLoS One. 2013; 8(9):e72944), including inhibiting EpH4-MEK-Bcl2 breast tumor growth in mice via induction of apoptosis (Tatarov I, et al., Comp Med. 2011; 61(4):339-345). EMFs have started to be used in conventional cancer treatment. For example, the US Food and Drug Administration approved the use of low-frequency alternating electric fields for the treatment of refractory glioblastoma multiforme (known as tumor treating fields) and other cancers (Stupp R, et al., JAMA. 2017; 318(23):2306-2316; Rick J, et al., J Neurooncol. 2018; 137(3):447-453).

In the current study, we explored dose-dependent changes of the cell biology of PDAC cells with respect to cell proliferation at the beginning of the study. Although we noted that 5 minutes of BT had a minimal effect on the growth of PANC-1 cells in two separate experiments using two different biofield therapists (FIG. 4A and FIG. 7), the inhibitory effect of BT on the growth of PANC-1 cells treated by two different therapists at a dose of 15 and 30 minutes appeared to be similar, and the 15-minute dose may even be stronger than the 30-minute dose. In fact, 15 minutes of BT led to stronger inhibitory activity on the cell growth of various PDAC cells, including PANC-1, L3.7, and COLO357 cells (FIGS. 4Aa, 4Af, 4Ag, and 4Ah), than did 30 minutes of BT, suggesting a nonlinear relationship between the duration of the treatment and the growth of PDAC cells. This is similar to what has been shown with the use of low-level light therapy (Huang Y Y, et al., Dose Response. 2009; 7(4):358-383). The mechanism contributing to this nonlinear dose-response relationship between BT and PDAC cells is unclear and warrants further study.

In conclusion, our data suggest that BT exerts antitumor activity against pancreatic cancer cells and PDOs in culture and their relevant orthotopic xenograft models. The in vitro studies suggest that BT modulates important cell cycle regulators such as FOXM1 and p21 and results in cell cycle arrest and cell hyperpolarization. Furthermore, we showed for the first time that BT treatment alters mitochondria ultrastructure and has the potential to reduce metastasis from pancreatic cancers.

### Example 2

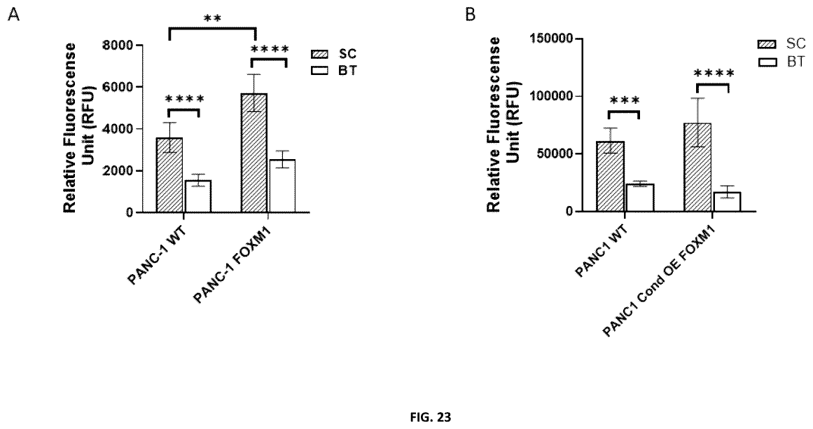

The methodologies and materials as set forth in Example 3 were used to conduct the following studies and to obtain the following insights and results, as further detailed in FIGS. 23-38. Additional methods are provided as follows.

For the KPCY model, we used pathogen-free female C57BL/6 mice (6 to 8 weeks old) with body weight 22±5 g, purchased from the Jackson Laboratory. Each mouse received an intrapancreatic injection of KPCY cells at 1×10⁵ cells per 50 μL of Hank balanced salt solution. Ten-fourteen days after injection, all animals injected with orthotopic tumor cells were imaged using a magnetic resonance scanner in the Small Animal Imaging Facility at MD Anderson Cancer Center) per standard protocol for pancreatic cancer imaging in mice.

Mice with tumor size 1-10 mm² were grouped into four groups (n=16 mice per group): 1) BT treatment group, with treatment provided 30 minutes per day, three times per week, for 3 weeks; 2) SC group, which received the same treatment schedule as the BT group but a sham therapist mimicked the movements and distance from the animals used by the biofield therapist; 3) colony control (CC) group, in which the animals stayed in the animal facility the whole time; and 4) gemcitabine-positive control group, which was treated with gemcitabine and also stayed in the animal facility the whole time. Both BT and SC groups (3-5 mice per group) were taken from the animal colony to the laboratory in their home cages, where they underwent the treatment sessions on a bench maintained in their home cages. For the gemcitabine-positive control group, gemcitabine was injected intraperitoneally at 40 mg/kg in saline three times per week and mice were maintained in the home colony. Similarly, mice from the CC group were injected with the same volume of saline intraperitoneally three times per week and maintained in the home colony.

Body weight of each mouse was measured once a week. The mice were euthanized three days after the treatment according to standard protocol. Primary tumors were collected and weighed, and final tumor weight were recorded. The nodules in the liver tissues were examined macroscopically and the intensity of YFP+ image of the liver was determined by imaging the mice with IVIX Spectrum instrument in Small Animal Facility of MDACC. Both tumor and liver tissues were either flash-frozen or fixed in 10% formalin for further analysis.

### Cell Free DNA.

Cell Free DNA was measured with NucleoSpin cfDNA XS kit (Macherey-Nagel, Allenton, PA). Briefly, 240 μl plasma or alternative cell-free fluid was transferred to a microcentrifuge tube. The biological samples that were of low volume were adjusted with the binding buffer. An aliquot of 20 μl of Proteinase K was added to the plasma and the mixture was incubated at 37° C. for 10 minutes. Following the incubation, 360 μl of binding buffer was added. The samples were then mixed by inverting the tube 3 times followed by a brief vortex (3s). This mixture was loaded into a Nucleospin cfDNA XM column with a collection tube and centrifuged at 2000×g for 30 s. The column was washed with 500 μl of washing buffer followed by a second wash of 250 ul washing buffer. Samples were centrifuged at 11,000×g for 30 s each time. The cfDNA was eluted with elution buffer (20-30 μl). The elute was incubated at 90° C. for 8 mins to remove the residual ethanol. The absorbance was measured with a microplate reader at 260 nm/280 nm (Molecular Device, San Jose, CA).

### Expression of Human Voltage-Gated Ion Channel Gene in PANC-1 Cells.

The expression of voltage-gated ion channel gene was first measured with TaqMan Array of Human Voltage-Gated Ion Channel (Catalog no. 4418744, Thermo Fisher) followed with validation with individual gene primers.

Briefly, the 96 well plates were pre-configured/pre-coated with human ion channel gene expression primers and target genes from all 4 classes of voltage gated ion channels: 1) Voltage-gated potassium channels, 2) Sodium channels, 3) Voltage-dependent calcium channels, and 4) Cyclic nucleotide-gated potassium channels. PANC-1 cells were subjected to RNA extraction using an RNA mini kit (Life Technologies, cat. #12183018A). Voltage-gated ion channel genes were measured with the TaqMan Array human voltage-gated ion channel (#4414099, Thermo Fisher) according to the manufacturer's instructions (https://www.thermofisher.com/order/catalog/product/4414099). Real-time PCR was performed on a QuantStudio 3 Real-Time PCR System (Applied Biosystems, Waltham, MA). Two separate plates were run for control and BT treated samples.

To further validate the changes of the voltage-gated ion channel gene, individual primer sets were purchased from Applied Biosystems/Life Technologies for the selected ion channel genes, including: Hs00240655_m1: CACNA1A; Hs00184168_m1 CACNA1I; Hs00185764_m1: KCNAB1; Hs00428197_m1: KCNC1; Hs00158410_m1: KCNG1; Hs00608142_m1: KCNH1; Hs00158470_m1: KCNN4; Hs00704943_s1: KCNA3 (kv.1.3); and Hs00165693_m1: SCNA5. Details of these primer sets can be found in the suppliers' website (https://www.thermofisher.com/us/en/home/brands/applied-biosystems.html). Realtime PCR were run using individual primers sets with RNA (20 ng/well) samples in triplicates and TaqMan Fast Virus 1-Step Master Mix (Applied biosystem).

### BT Inhibited Migration and Invasion in PDAC Cells.

As described in Example 1 and shown in FIG. 13, we examined the effect of BT on the invasiveness of PDAC cells. Our further studies showed BT also led to significant reduction of migration in PANC-1 and L3.7 cells, by 34% and 41%, respectively, compared with SC (p<0.001; FIG. 13).

### BT Altered EMT Related Markers in PANC-1 Cells and Reduced the Cell Free DNA in PANC-1 Tumor Model.

To understand how BT suppressed the invasiveness of the PDAC cells, we examined EMT related marker expression in PANC-1 cells. As described in Example 1 and shown in FIG. 18, the protein expression of E-cadherin, a marker of epithelial cells, in PANC-1 cells exposed to BT was significantly higher than in the IC and SC groups (FIG. 18Aa). In contrast, the protein expression of N-cadherin and gene expression of Twist, markers of mesenchymal cells, were significantly lower in the BT group than in the IC and SC groups (p<0.05; FIGS. 18Ab and 24). Additionally, the level of cell-free tumor DNA was significantly lower in BT treated mice compared to that of SC mice bearing PANC-1 orthotopic tumor (FIG. 24C), suggesting that BT can potentially slow down the intravasation process, which is line with the results of BT on inhibition of PDAC cell migration and invasion.

### BT Altered the Ultrastructure of PANC-1 and L3.7 Cells.

We examined the effect of BT on the ultrastructure of the PANC-1 and L3.7 cells. The ultrastructural changes in cellular organelles were measured immediately after BT treatment of PANC-1 and L3.7 cells using Transmission Electron Microscope (TEM). As described in Example 1 and shown in FIG. 8, PANC-1 cells in the IC and SC groups had elongated mitochondria with well-organized cristae. In contrast, PANC-1 cells exposed to BT had disordered mitochondrial cristae and the mitochondria were markedly swollen. Similar mitochondrial changes, i.e., losing the cristae integrity and noticeable swelling of the mitochondria, were observed after 15 (FIGS. 25A, 25B) and 30-minute of BT treatment in repeated studies using PANC-1 cells (FIG. 25B). Additionally, the cytoskeleton structure of BT treated L3.7 cells was substantially less noticeable than the cells from IC and SC groups (data not shown). This profound and visually obvious morphology changes in the mitochondrial and cytoskeleton ultrastructure not only substantiates but also provides new insight into the biological action of BT on important cellular function.

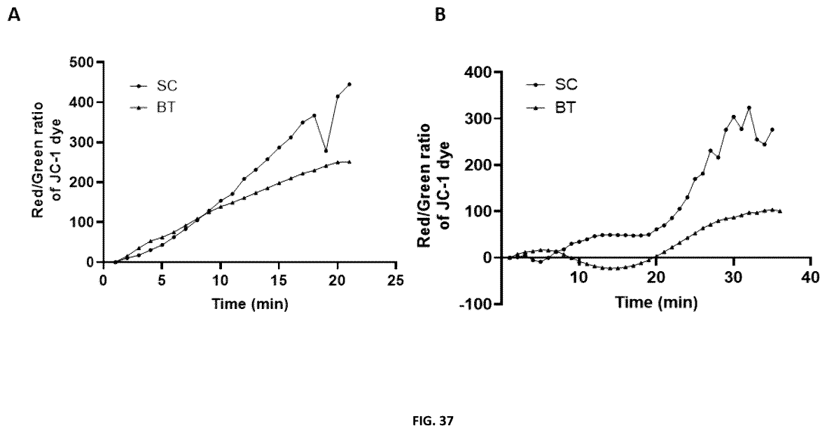

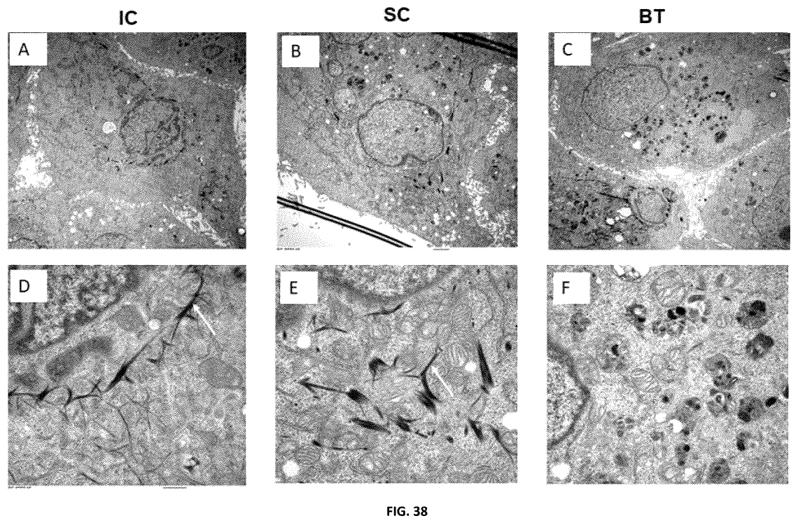

For example, as shown in FIG. 37, we found that BT treatment reduced the ratio of mitochondria stained with fluorescence red and green JC-1 dye compared to that of sham control group, suggesting BT can potentially decrease the mitochondrial membrane potential in PANC-1 cells. As shown in FIG. 36, the epigenetic testing showed that the BT treatment modifies the openness of the chromatins related to cytoskeleton structure, serine and threonine kinase, cell junction, extracellular matrix and cell cycle in PANC-1 cells, suggesting BT treatment may transcriptionally regulate these genes. As shown in FIG. 38, the TEM examination showed that BT treatment reduced the cytoskeleton filamentous structure in human pancreatic cancer L3.7 cells compared to that IC and SC group, suggesting BT treatment may alter the mechanical properties of the cells.

### Modulation of FOXM1 Expression Altered the Response of PDAC Cells to BT.

As discussed in Example 1 and shown in FIG. 19, the stronger the suppressive effect of BT on the expression of FOXM1 the greater the inhibition of BT on cell invasion. This suggests that BT-induced anti-invasiveness in PDAC cells might be at least in part mediated by its ability to downregulate FOXM1 protein.

We then examined the cell cycle and invasiveness of control and FOXM1-modified PDAC cells after BT or SC. In the FOXM1 stable knockdown (KD) PANC-1 cells, the expression of FOXM1 was reduced by almost 90% compared with that of parental PANC-1 cells (FIGS. 19, 20, and 26). In the FOXM1 conditional overexpressed PANC-1 cells, while BT treated parental PANC-1 cells exhibited 60.5% less invasiveness than that of the SC group, BT led to greater inhibition of invasiveness in the conditional FOXM1 overexpressing PANC-1 cells (77.6%) compared to that of SC group (p<0.001) (FIG. 23B). Similar changes were also observed in FOXM1 stably overexpressing PANC-1 cells after being treated with BT (FIG. 23A).

### BT Treatment Altered the Voltage Gated Sodium and Potassium Channels and FOXM1 Appears to be Involved in the Cell Voltage Potential.

Given we previously consistently observed that BT altered cell voltage potential, and voltage gated ion channels (VGIC) are major regulators for the resting cell voltage potential, we examined the gene expression of VGIC, including sodium (VGSC), potassium (VGKC) and calcium (VKCC) ion channels in BT treated PANC-1 cells. As can be seen in FIG. 27, more than 10 genes in VGKC were down-regulated in the BT treated PANC-1 cells compared to that of SC group and the expression of two genes of VGSCs, including Nav1.1 and Nav1.5, were lower in BT treated PANC-1 cells (FIG. 27A). Only a moderate effect in the gene expression of VGCC were observed (data not shown). Real time PCR confirmed that the gene expression of Nav1.5 was significantly increased in 15 min BT treated PANC-1 cells whereas it was significantly reduced in 30 min BT treated PANC-1 cells compared to that of SC group (FIG. 27B). In contrast, the levels of Kv1.3 genes were moderately, but significantly lower in 15 min BT treated PANC-1 cells, but significantly higher in 30 min BT treated samples compared to that of SC group (FIG. 6C). Considering Nav1.5 is mainly responsible for Na+ influx and Kv1.3 regulates K+ influx, this data suggest that BT treatment leads to depolarize the cells followed with potential hyperpolarization of the PANC-1 cells.

Additionally, we also observed BT significantly downregulated gene expression of Kca3.1 in PANC-1 cells (FIG. 27D). Considering BT-induced anti-invasive activity appears to be partly mediated by FOXM1, we examined whether FOXM1 was involved in cell membrane voltage potential. We measured the resting membrane voltage potential of the PANC-1 and FOXM1 CRISPR FOXM1 KO PANC-1 cells with DiBAC4 staining after 15 min treatment. Interestingly, FOXM1 KO PANC-1 cells exhibited significantly higher resting cell voltage membrane potential than that of parental PANC-1 cells (FIG. 27E), suggesting reduced FOXM1 expression is associated with the depolarization of PANC-1 cells, which is consistent with cell membrane voltage changes in 15 min BT treated PDAC cells.

### The Antitumor Activity of BT was Confirmed in PANC-1 Mouse Orthotopic Model and KPCY Mouse Syngeneic Model.

To further confirm the antitumor effect, especially anti-metastatic potential of BT on pancreatic cancer, we repeated the antitumor efficacy study previously conducted in human PANC-1 mouse orthotopic model and further evaluated the antitumor efficacy in a mouse KPCY syngeneic model. The experimental procedure was similar to that used in our previous in vivo PANC-1 mouse orthotopic model.

The average terminal tumor weight over body weight of mice in all treatment groups was similar (FIG. 28A) which was slightly different from the result of the first study with PANC-1 orthotopic model. In contrast, and similarly to the results described in Example 1 and shown in FIG. 21, we observed that 28% of the mice in BT group had liver metastasis, which was 51% less than that of CC group, 55% less than the SC group, and 53% fewer than the Gem group (FIG. 28B). Strikingly, when the liver tumor burden in the mice with liver metastasis was examined macroscopically, the average tumor burden of the mice treated with BT was 8.1±5.4 mm3, which was 73% less than that of SC group (26.0±6.2 mm3) (p<0.05). Mice treated with gemcitabine only moderately reduced liver tumor burden compared to the CC group (FIG. 28C). While there were more and larger nodules in the liver of CC and SC mice, a normal looking liver was present in the BT treated mice (FIG. 28D). Similarly, BT significantly reduced the visible liver mets in mice bearing KPCY T-cell low clone (6419C5) tumors (35%) compared to that of CC (92%) and SC (79%) groups (p<0.01, FIG. 28E). When we imaged the YFP+6419C5 cells in the liver tissues with IVIS instrument, we noted that the intensity of YFP+ cells in BT treated liver tissues were statistically significantly lower than that of SC group (FIG. 7F). The image of liver tissues showed higher YFP+ intensity of the liver mets in SC group compared to that of BT treated mice (FIG. 28G). Together, these data strongly suggest that BT is efficacious against the metastasis to the liver in two different orthotopic pancreatic cancer models—PANC-1 and KPCY.

BT treatment Altered Immune Cell Profile in KPCY Mouse Pancreatic Tumor.

Even though we did not see significant survival benefit of BT in mouse pancreatic KPCY high T-cell clone (7160C2) derived tumor bearing mice, we carried out a pilot study to examine the effects of BT on the TME. As shown in FIG. 29, BT treatment significantly reduced the CD19+ B cell (FIG. 29A) and increased the CD3+ T-cell (FIG. 29B) infiltration in the KPCY 7160C2 derived tumors compared to that of SC control group. When the subtype of T-cells was evaluated, we found that CD8+ cytotoxic T-cells in BT treated tumors were 2.74- and 1.58-fold higher than that of CC and SC groups, respectively (FIG. 29C). In contrast, the mean percentage population of FOXP3+CD25+ T-Regulatory cells in the BT treated tumors was similar to that of CC groups and slightly lower than that of SC group (FIG. 29D). Considering the CD8+ cytotoxic T-cells functions to kill cancer cells and T-Reg cells stimulate tumor growth, this preliminary data provides strong rationale to conduct in depth evaluation of whole immune cell landscape in BT treated pancreatic tumor.

### Faraday Cage, Aluminum Foil, and Distance Did Not Block the Effect of BT on Cell Cycle and Cell Invasion of PDAC Cells.

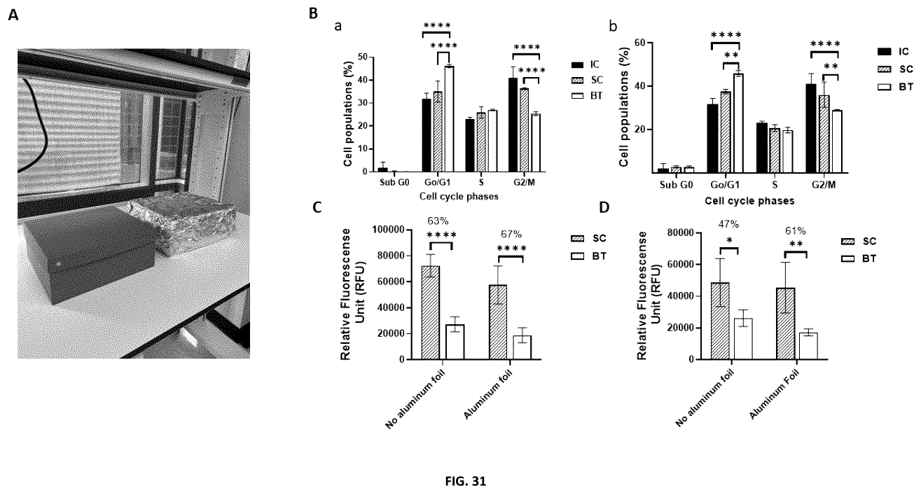

To gain more insights on the potential mechanisms of BT, we examined the cell cycle and cell invasion of PDAC cells when they were placed in the EMF shielding devices such as Faraday cage or wrapped in aluminum foil. For Faraday cage testing, PDAC cells were either placed on the bench or inside of a faraday cage (Holland Shielding System BV, Netherland, FIG. 30A). Prior to placing the cells inside faraday cage, the EMF shielding effect was calibrated with blocking the signal of cell phone. The effect of aluminum foil on BT induced anti-invasiveness was also tested to gauge whether it is possible to attenuate or block the effect with lengths of aluminum foil commonly produced by industry. Aluminum foil is manufactured in such a manner that is said to result in molecular spin ordering, forming an aligned unidirectional structure. For aluminum foil testing, the sham control PDAC cells were either placed on the bench inside a plain shoe box or inside a similar box but with two layers of aluminum foil lining inside and the outside of box with the layers placed at a 90 degree angles and the shiny sides back-to-back (FIG. 31A) in order to create a polarizer to potentially block any signal which may have spin or chirality.

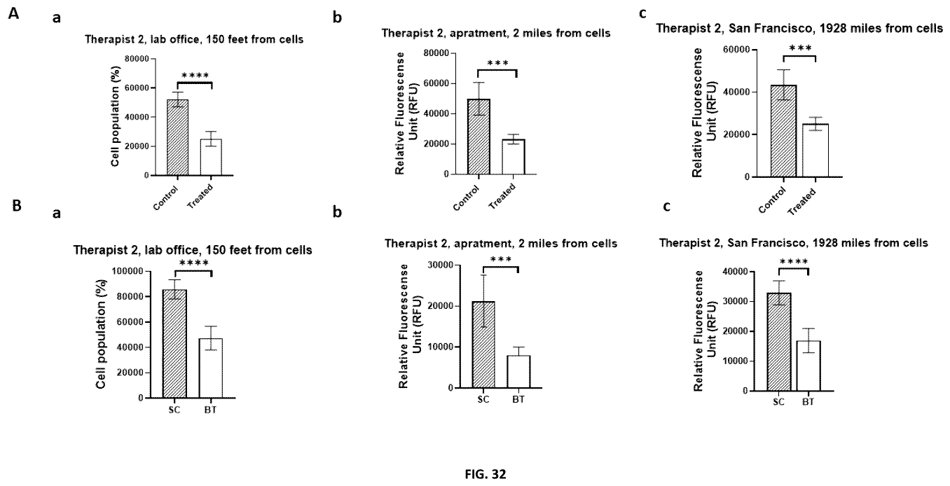

As shown in FIG. 30, BT induced G1 phase arrest evidenced by higher G0/G1 phase (44.5%) in BT treated PANC-1 cells placed on the bench compared to that of IC (38.67%) and SC (39.26%) groups (p<0.001 and 0.01, respectively, FIG. 30Aa). Similar outcomes were observed in the cells placed inside the faraday cage (FIG. 30Ab). When the cell invasion of the PANC-1 and L3.7 cells was measured either placed on the bench or inside of faraday cage, the inhibition of cell invasion by BT in PANC-1 and L3.7 cells inside the faraday cage were similar to that of cells placed on the bench (FIGS. 29B and C). Similar changes were observed on the cell cycle and anti-invasiveness of BT treated PANC-1 and L3.7 cells on the bench either placed inside a box with or without aluminum foil (FIG. 31). Moreover, we measured the cell invasion of the PANC-1 cells when BT was conducted via Zoom from three different distances from the bench where the cells were placed: 1) from the lab office 150 feet away; 2) 1.5 miles away; and 3) when the BT was conducted from San Francisco Bay area. These series of studies were conducted with two different therapists with control samples assessed simultaneously in a different part of the laboratory. Distance did not change the effects of BT, as can be seen in FIG. 32. These data strongly suggest that EMF is not directly involved in BT-induced antiproliferative and anti-metastasis potentials in PDAC cells and non-EMF mechanism might be involved.

BT Altered Intracellular Calcium Signaling and Beta-Actin that Correlated with Changes in rhe EEG of the Therapist.

Another exploratory study was conducted to understand the mechanism of BT by simultaneous assessment of changes in cellular processes and changes in the therapists biometrics. In this study, multiple treatment and control (non-treatment) sessions (n=60) were carried out in which the therapist was blinded to the conditions. The experimental setting is shown in FIG. 33. During the treatment phases, the therapist attempted to modify in vitro cell outcomes, alternating with rest phases where no such efforts were made. Human pancreatic cancer cell activity was assessed using three markers-two cytoskeleton changes (tubulin and β-actin) and Ca2+ uptake. Given we observed some EEG changes with the therapist when the treatment was carried out during the Phase 1 studies, the goal of the study was to explore the changes in the therapist's physiological parameters (electroencephalogram (EEG)) and heart rate measures during the treatment of 1) live cells and 2) either dead cells or no cells/medium only (control group), changes in cellular outcomes, and if there was an association between the therapist's physiological parameters and cellular outcomes. The experimental setup was a 2×2 design, contrasting cell type (live vs. control) against session type (treatment vs. non-treatment). Parallel sham-treated control cells were examined for changes in the cell parameters over time and cell invasions assay was set up as a positive control. The therapist's physiological data, including 64-channel EEG and heart rate, were continuously monitored throughout these sessions.

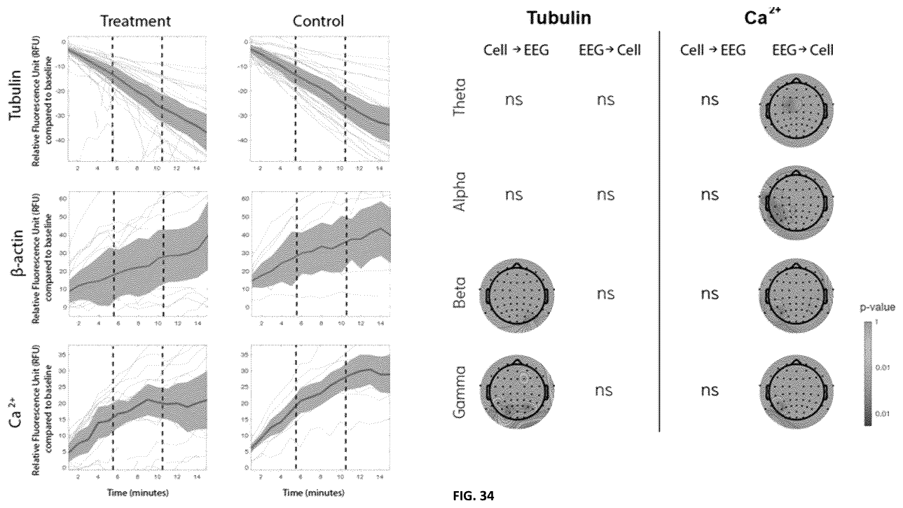

We observed significant (p<0.01) spectral changes in the therapist's EEG during BT treatment in all frequency bands of interest, as well as in heart rate variability (HRV) (RMSSD; p<0.01). We also observed significant differences in beta and gamma EEG and HRV (pNN50) when the therapist treated live but not control cells (p=0.02). However, no interaction between treatment and cell type (live vs. dead cells/medium) was observed. We observed Ca2+ uptake increased over time during both BT and sham treatment, but the increase was significantly less for the BT group compared to the sham-treatment controls (p=0.03, FIG. 34A). When using Granger causality to assess causal directional associations between cell markers and therapist's physiological parameters, EEG measurements showed significant bidirectional causal effects with cell metrics, especially β-actin and Ca2+ uptake (p<0.000001, FIG. 34B). Even though we did not observe any quantum entanglement due to the limitation of technology challenges (millisecond measurements of EEG but only minute-to-minute measures for the cell measures), these outcomes suggest a complex relationship between physiological responses and cellular effects during BT treatment sessions, and follow-up studies are warranted.

### The Antimetastatic Effect of BT was Confirmed by the Second Biofield Therapy.

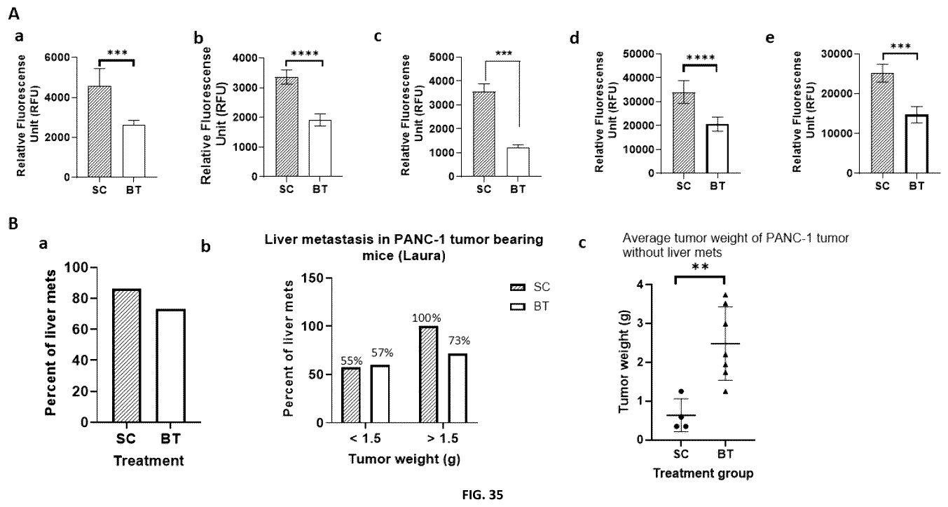

In order to test the hypothesis that a second BT has a similar effect on in vitro experiments and in vivo animal model, we carried out the studies to examine whether a second biofield therapist has similar outcomes in invasiveness and migration of the PDAC cells and human PANC-1 mouse orthotopic model. As shown in FIG. 35A, the anti-invasiveness and anti-migrative effects of BT in these PDAC cells treated by the Therapist 2 were similar to those observed with Therapist 1. Compared to the result of in vitro studies, the PANC-1 mouse orthotopic model mice treated with BT had only a moderate reduction in the liver metastasis compared to that of SC group (FIG. 35Ba). However, when comparing the incidence of liver mets based on the primary tumor weight in tumor bearing mice, the mice with larger tumors in the BT treated mice had 27% less liver nodules than that of SC group (FIG. 35Bb). In fact, size of primary tumor in the mice with no liver metastasis of the BT treated mice were significantly larger than that of SC group (FIG. 35Bc), suggesting that BT treatment indeed slows down the liver metastasis in the PANC-1 animal model. Together, these data indicate replicability of these findings across studies and therapists.

### Example 3

### Materials and Methods

### Reagents and Antibodies.

All procedures were performed according to the relevant guidelines, rules, and regulations of The University of Texas MD Anderson Cancer Center. PrestoBlue was purchased from ThermoFisher; an invasion kit and cell migration assay kit from Cell Biolabs, USA; DiBAC4 (bis-[1,3-dibutylbarbituric acid] trimethine oxonol) (B438) from ThermoFisher; and gemcitabine from MD Anderson Pharmacy. Antibodies against FOXM1 (5436), p21 (2947), N-cadherin (13116), and E-cadherin (14472) were purchased from Cell Signaling Technology (Danvers, MA) and anti-actin (MAB8929) were purchased from R&D Systems. For running the Jess system, 12-230 kDa Separation Module, 8×25 capillary cartridges (SM-W004), Anti-Rabbit Detection Module (DM-001), and Anti-Mouse NIR Detection Module (DM-009) were purchased from ProteinSimple (CA, USA). Nucleus live cell staining dye Invitrogen NucBlue Live ReadyProbes Reagent (Hoechst 33342, R37605) was purchased from ThermoFisher.

### Cell Lines.

Human pancreatic cancer cells (PANC-1 and MiaPaCa-2) were purchased from the American Type Culture Collection (Manassas, VA), and the human pancreatic cancer cells COLO357 and L3.7 were used as described previously (Huang C, et la., Cancer research. 2012; 72(3):655-665). Mouse pancreatic cancer KPCY cells were purchased from Kerafast (Boston, MA). All cancer cells were maintained in a humidified atmosphere with 5% carbon dioxide at 37° C. PANC-1, COLO357, and L3.7 cells were routinely cultured in either Dulbecco modified Eagle medium with high glucose or RPMI 1640 medium (Invitrogen Corp, Grand Island, NY) containing 10% fetal bovine serum (Hyclone Laboratories Inc, Logan, UT) supplemented with 50 IU/mL penicillin, 50 μg/mL streptomycin, and 2 mM L-glutamine from GIBCO (Invitrogen). All human cell lines were authenticated using short tandem repeat profiling within the past 6 months (available on request).

### BT Procedures for In Vitro Studies.

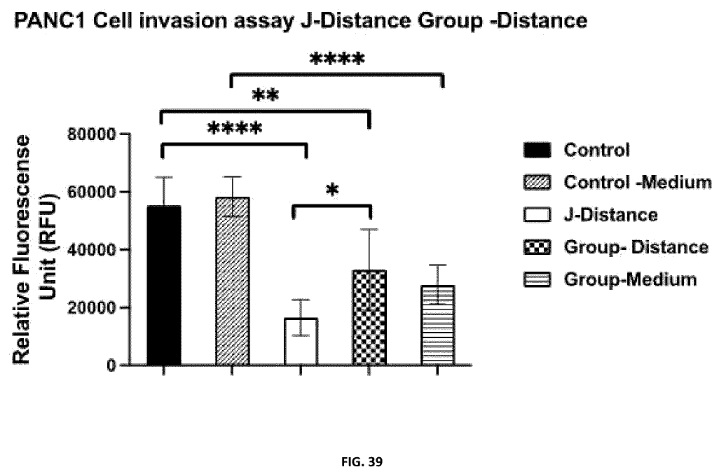

Most of the experiments were conducted by one therapist, and some of the cell experiments were replicated with one or two other therapists (indicated as Therapist 2 and/or Therapist 3 in the figures). In one experiment, BT therapy was administered from a distance by a group of therapists; as shown in FIG. 39, we found that PANC-1 cells treated with individual therapist or group therapists exhibited the similar reduced capacity to invade compared to that of sham control group, which support the hypothesis of BT indeed can potentially inhibit the invasion of PANC-1 cells.

Initial cell experiments explored a possible dose-response effect and examined exposures of 5, 15, and 30 minutes, as well as 60 and 120 minutes (data not shown) of BT. There was no clear indication of a dose-response effect, and, depending on the cell line, the 15- and 30-minute exposures had the strongest effect and were used for most of the experiments. Unless otherwise indicated, treatments were provided for 15 minutes. Each study had two or three groups: 1) BT treatment group, where cells were either on the counter or treated through an incubator; 2) sham counter control (SC), where the plated cells were on the counter for the same amount of time as in the BT group and a person mimicked the movements and distance from the cells as in the BT group; and 3) for some experiments, plated cells remained in the incubator (incubator control [IC]). The pathologist was blinded to group assignment when conducting the assays or evaluating the animals.

### BT Protocol.

The BT protocol included among other techniques the Bengston healing energy method. This involves creating images that the therapist cycles through as rapidly as possible while using a hands-on technique. Detailed descriptions of these rapid imaging and hands-on techniques have been described previously (Bengston V F, Kinsley, D. Journal of Scientific Exploration. 2000; 14(3):353-364; Bengston W F, Moga M. J Altern Complement Med. 2007; 13(3):317-327). Briefly, while the therapist cycles through the images, they are in a seated position in front of the target receiving the treatment (petri dishes or animal cages). They are in a relaxed state with their hands approximately 6 to 24 inches from the target and may engage in slow movements with their hands, but never touching the dishes/cages/animals. In the experiments using sham, a sham therapist mimicked the movements/distance from the cells/animals used by the biofield therapist.

### Transmission Electron Microscopy.

Transmission electron microscopy was used for detection of fine details of organelle structure within cells and was performed in the High-Resolution Electron Microscopy Facility (HREMF) at MD Anderson Cancer Center. PANC-1 cells (1×10⁵) were plated in 12-well plates and then exposed to BT for 15 minutes. Immediately after treatment, cells were washed with PBS (without Ca⁺⁺/Mg⁺⁺) prior to treatment with EM fixative containing 3% glutaraldehyde and 2% paraformaldehyde in 0.1M cacodylate buffer (pH 7.3). Samples were then further processed according to the standard operating procedure of HREMF and procedures published previously (Yang P, et al., Prostate. 2003; 55(4):281-291).

### Cell Migration and Invasion.

Colorimetric cell invasion assay was performed using Cytoselect. The 24 wells basement membrane kit (Cell Biolabs, #CBA-110). Briefly, PANC-1 cells suspension (1.0×10⁶, 300 μl) were plated in polycarbonate inserts/upper chamber with basement membrane (8 μm pore size) in DMEM without FBS whereas the bottom chamber contained 500 μl DMEM with FBS. Control cells and treated cells were plated in two different plates. After BT (15 min or 30 min), the cells were continually incubated for 48 hours at 37° C. in presence of 5% CO2. The media were aspirated carefully from the inserts after 48 hours and the inserts were transferred to clean wells containing 400 μl of staining solution from the kit for 10 minutes followed by gently washing with clean water. They were then air dried and images of invading cells were taken by EVOS M7000 Imaging System (Thermo Fisher Scientific).

Cell migration assays were done using Cytoselect cell migration assay kits (Cell Biolabs, USA) in 24-well plate format. Next, 1.0×10⁶ cells in 300 μL medium without fetal bovine serum were plated in each insert (6 inserts/group). An aliquot of 500 μL of media containing 10% fetal bovine serum or desired chemoattractant(s) was used in the lower well of the migration plate. The plates were then exposed to BT (15 minutes) followed by incubation for an additional 24 hours in a humidified CO₂ incubator. The inserts were processed as described in the manufacturer's protocol. Similarly, the cell invasion assay was conducted using the Cell Biolabs CytoSelect Cell Invasion Assay kit (24-well format), which uses basement membrane-coated inserts to assay the invasive properties of tumor cells. The endpoint was monitored 48 hours after BT. The fluorescence intensity was measured with a microplate reader at 480 nm/520 nm (Molecular Device, San Jose, CA).

For the scratch assay, the Culture-Insert 2 Well in μ-Dish 35 mm (Ibidi, Fitchburg, WI) was used. The dish has culture-inserts to create a reproducible gap size. MiaPaca-2 cells (6×10⁵) were seeded and cultured in each of the two culture-insert wells for 24 hours. The insert was then gently removed by sterile tweezers and an aliquot of 1 mL of culture medium was added to the culture dishes. The cell culture dishes were placed in two separate CytoSmart FL microscopes (Axion Biosystem, Atlanta, GA) inside two incubators, and the cell culture dishes were exposed to sham control or BT. Images were captured every 30 minutes for 48 hours. The images were analyzed by CytoSmart scratch assay online program.

### Cell Voltage Characterization.

Briefly, cells (5-10×10³) were plated the day prior to treatment. After the treatment, plates were cultured for 5 days in the incubator at 37° C. in 5% CO₂ and 95% humidified air. DiBAC4(3), a slow-response potential-sensitive probe, was used to stain and measure the depolarization of the cells. The working DiBAC4(3) solution (1 mg/mL) was prepared by mixing the stock solution with Dulbecco modified Eagle medium—free fetal bovine serum at a ratio of 1:4000. Cells were washed with PBS and DiBAC4(3) working solution together with 1 drop of NucBlue Live Ready Probes Reagent and incubated for 15 minutes at 37° C. Green fluorescent protein and DAPI images were captured by the EVOS M5000 Imaging System (AMF5000) or EVOS M7000 Imaging System (AMF7000). Fluorescence intensity was quantified by ImageJ for the images captured by EVOS M5000 or by Cellslate software for the images captured by EVOS M7000.

### Immunoblotting.

Immunoblotting was performed using an automated Western blotting system: the Jess Simple Western system (ProteinSimple, San Jose CA, USA) automatically separates and immunodetects proteins by size through its capillaries. To quantify the protein expression of interest in the treated cells, we followed the standard method for the 12-230 kDa Jess separation module (SM-W004). The final protein concentration of tissue lysate (0.4 to 1 mg/mL) was obtained by mixing measured cell lysate (0.6 to 2 mg/mL) with 0.1X sample buffer and Fluorescent 5X Master mix (ProteinSimple). This mixture was denatured at 95° C. for 5 minutes. Primary antibodies of interest are indicated in the Results section, and β-actin or tubulin were prepared by mixing antibody diluent at a ratio of 1:10 to 1:50. Secondary antibodies were prepared by combining chemiluminescence antibody and fluorescence antibody. After that, cell lysate samples were loaded into the Ladder plate (12-230 kDa), followed by loading of antibody diluent, primary antibody, secondary antibody, a luminol-s and peroxide mixture (for chemiluminescence detection), and wash buffer according to the manufacturer instructions. Digital images of chemiluminescence and fluorescence for the proteins of interest were captured with Compass Simple Western software (version 4.1.0, Protein Simple), and the area was used for quantification of the proteins.

### FOXM1 Gene Modification.

To establish FOXM1-overexpressing PANC-1 cells, we used hFOXM1 human ORF clone lentiviral particles purchased from OriGene USA to overexpress the FOXM1 gene in PANC-1 cells. Lentiparticles were thawed on ice and added to 6-well plates directly in the medium (500 μL) along with Polybrene (final concentration 8 μg/mL), and the lentiparticles were then incubated for 20 hours at 37° C. in a humidified CO₂ incubator. To generate stable cell lines, we plated transduced cells at low densities in a fresh 6-well plate, and the clones were selected with puromycin (50 μg/mL). Single colonies were picked and expanded further and characterized using Jess/Western blot.

To develop the FOXM1 knockdown stable PANC-1 cell lines, we transfected three sets of human FOXM1 hCMV-TurboRFP shRNA lenti-viruses (Horizon Discovery Bioscience) into PANC-1 cells. After transfection for 48 hours, puromycin (10 μg/mL was used for the colony selection for at least 2 weeks. The colony characterized with downregulated FOXM1 was then cultured with 5 ng/mL puromycin for all additional experiments. hFOXM1 knockdown was confirmed by Western blot (Jess analysis), and PANC-1 cells that were most efficiently knocked down on FOXM1 were chosen for the experiments.

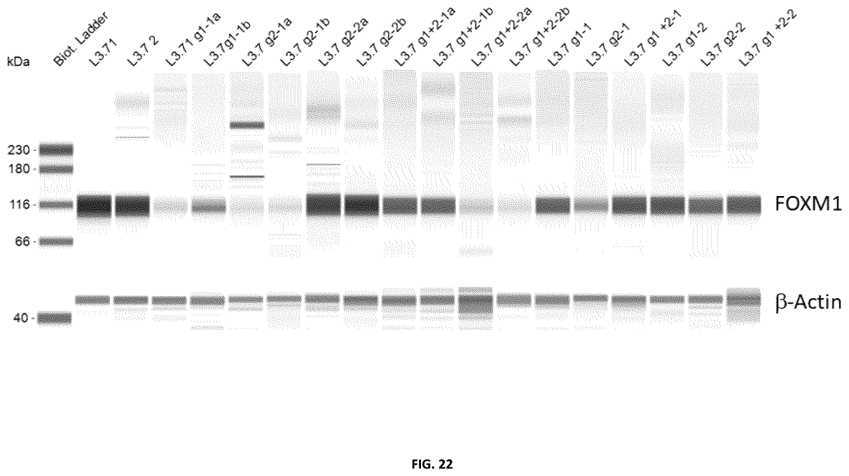

In some experiments, FOXM1 knockout L3.7 cells were used, which were generated by CRISPR-Cas9 transfection. Briefly, L3.7 cells were plated in 6-well plates and allowed to attach overnight. The transfection of a scramble control vector or a FOXM1 knockout gene kit via CRISPR-Cas9 (OriGene, Rockville, MD, cat. #KN402246) was carried out using TurboFectin Transfection Reagent (OriGene, cat. #TF81001) following the manufacturer's instructions. Briefly, 1 μg of one of the gRNA vectors, gRNA1 or gRNA2 in 250 μL of Opti-MEM I (Life Technologies), in separate tubes, was vortexed gently. Then 1 μg of the donor DNA was added into the same 250 μL of Opti-MEM I. An aliquot of 6 μL of Turbofectin 8.0 was added to the diluted DNA and pipetted gently to mix completely. The mixture was incubated for 15 minutes at room temperature. The mixture above was added dropwise to the cells plated in 6-well plates. The plate was gently rocked back and forth and side to side to distribute the complex evenly. Following that, cells were incubated in a 5% CO₂ incubator. Then 48 hours after transfection, cells were split at 1:10, grown for an additional 3 days, and split again at 1:10. This procedure was repeated seven times in total. After passage 7, puromycin was added and expression was checked using Jess. The colonies with stable FOXM1 knockout (g2-1; FIG. 22) were selected with puromycin and by substantially reduced expression of FOXM1.

To establish the inducible FOXM1 expressing PANC-1 cells, the tetracycline inducible pCW57.1 HA-FOXM1b was purchased from Addgene. The inducible pCW57.1 HA-FOXM1b (5 mg) was transiently transfected to HEK293T cells in 10 cm plate, along with helper plasmids psPAX2 (6 mg) and 2 mg of pMD2G (Addgene: 12260, Addgene: 12260) using X-tremeGENE9 DNA (Roche) transfection reagent as per the manufacturer's protocol. Viral supernatants were collected by filtering through 0.45 mm filter (Millipore) after 48 hours of transfection. The PANC-1 cells were transduced with the viral supernatant in presence of Polybrene (4 μg/ml, Sigma) in serial dilutions in 10 cm plates. The plates were then treated with 0.5 μg/ml Puromycin 48 h post transfection and puromycin selection was continued for 5 days. After five days, cells were cultured without Puromycin for a week. To test the doxycycline (Sigma) inducible human FOXM1 expression in PANCI cells, the cells were plated in 6 well plates and treated with/without doxycycline (250 ng, 500 ng and 1000 ng). Media with or without doxycycline were changed every 24 hours. The expression of inducible FOXM1 was monitored using automated western blot system (Jess, Protein Simple) after 72 hours of doxycycline treatment.

### Animal Models of Pancreatic Cancer.

Animal studies were conducted according to MD Anderson Cancer Center Animal Care and Use Committee rules and regulations (IACUC protocol number: 00001212-RN02). Animals were housed in facilities approved by the Association for Assessment and Accreditation of Laboratory Animal Care International in accordance with the current regulations and standards of the US Department of Agriculture and Department of Health and Human Services. For tumorigenesis of human PANC-1 cells, we used pathogen-free female athymic BALB/c nude mice (6 to 8 weeks old) with body weight 22±5 g, purchased from the Jackson Laboratory. Each mouse received an intrapancreatic injection of PANC-1 cells at 0.5×10⁶ cells per 50 μL of Hank balanced salt solution. Ten days after injection, all animals injected with orthotopic tumor cells were imaged using a magnetic resonance scanner in the Small Animal Imaging Facility at MD Anderson Cancer Center) per standard protocol for pancreatic cancer imaging in mice.

Mice with tumor size 1-10 mm² were grouped into four groups (n=25-30 mice per group): 1) BT treatment group, with treatment provided 30 minutes per day, three times per week, for 3 weeks; 2) SC group, which received the same treatment schedule as the BT group but a sham therapist mimicked the movements and distance from the animals used by the biofield therapist; 3) colony control (CC) group, in which the animals stayed in the animal facility the whole time; and 4) gemcitabine-positive control group, which was treated with gemcitabine and also stayed in the animal facility the whole time. Both BT and SC groups (3-5 mice per group) were taken from the animal colony to the laboratory in their home cages, where they underwent the treatment sessions on a bench maintained in their home cages. For the gemcitabine-positive control group, gemcitabine was injected intraperitoneally at 40 mg/kg in saline three times per week and mice were maintained in the home colony. Similarly, mice from the CC group were injected with the same volume of saline intraperitoneally three times per week and maintained in the home colony.

The mice were treated for 3 weeks and then observed for another 7 weeks. Body weight of each mouse was measured once a week. At the termination of the study, mice were euthanized according to standard protocol, and blood, tumor/pancreas, spleen, liver, and lungs were collected and either flash-frozen or fixed in 10% formalin for further analysis.

### Immunohistochemical Analysis.

Liver tissue samples from mice were obtained at necropsy. Tissues were fixed in 10% neutral buffered formalin and subsequently embedded in paraffin. Sections were stained with hematoxylin and eosin using a standard protocol of the Histopathological Core Facility of MD Anderson. The stained slides were scanned with Aperio AT2 bright-filed slide scanner and examined by a certified pathologist in a blinded fashion.

### Statistical Analysis.

GraphPad Prism was used for the statistical analysis (t test or ANOVA) in both in vitro and in vivo studies. For RPPA data, statistical analysis was performed using R Statistical Software version 4.1.1 (R Foundation for Statistical Computing, Vienna, Austria) and Wilcoxon rank-sum tests. For these analyses, p values <0.05 were considered statistically significant. Data are presented as mean±SD unless otherwise indicated.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention. All publications mentioned in this application are herein incorporated by reference to the same extent as if each individual publication was specifically and individually indicated to be incorporated by reference.

1. A method of decreasing metastatic potential of cells containing mitochondria, the method comprising:
effecting a biophysical change, the biophysical change comprising at least two of:
altering intracellular ion levels of the cells,
altering cell membrane potential of the cells,
altering membrane potential of the mitochondria, and
altering of expression of ion channel genes;
effecting a structural change, the structural change comprising at least one of:
changing a structural feature of the mitochondria,
changing cytoskeleton organization of the cells,
altering membrane ion channels of the cells,
altering of expression of cytoskeletal genes, and
altering of expression of genes involved in communication with the cytoskeleton;
effecting a physiological change, the physiological change comprising at least two of:
altering one or more EMT marker proteins in the cells,
regulating FOXM1 in the cells,
altering energy metabolism in mitochondria of the cells,
altering of expression of one or more EMT genes,
altering of expression of genes for one or more FOXM1-associated proteins,
altering of expression of genes for serine/threonine kinases,
altering of expression of genes for cell junction, and
altering of expression of genes for extracellular matrix;
wherein the biophysical change, the structural change, and/or the physiological change are correlated with at least two of:
cell cycle arrest of the cells and/or expression of cycle regulation proteins,
decreased cell-free DNA from the cells or intravasation,
decreased growth or metabolism of the cells,
deceased migration of the cells,
suppressed stemness, and
decreased invasion of the cells.

2. (canceled)

3. (canceled)

4. (canceled)

5. The method of claim 1, further comprising:
administering a biofield therapy,
wherein administering the biofield therapy is correlated with at least one of: the biophysical change, the structural change, and the physiological change.

6. (canceled)

7. The method of claim 1, wherein
the biophysical change, the structural change, and/or the physiological change are correlated with decreased migration and/or invasion of the cells.

8. The method of claim 7, wherein
decreased migration and/or invasion of the cells is achieved without toxic effects to the cells or a host of the cells.

9. The method of claim 1, further comprising:
administering a chemical agent and/or radiotherapy.

10. (canceled)

11. (canceled)

12. (canceled)

13. (canceled)

14. The method of claim 5, wherein
a portion of the biofield therapy is administered by a group of therapists.

15. The method of claim 5, wherein
the biofield therapy is administered locally or at a distance.

16. The method of claim 5, wherein
administration of the biofield therapy is determined according to an indicator of efficacy of a person performing the biofield therapy or a manner of performance of the biofield therapy.

17. The method of claim 16, wherein
the indicator of efficacy is based on the biophysical, structural and/or physiological change or a measurement of brain electrical activity (e.g., an EEG) of the person performing the biofield therapy.

18. The method of claim 17, wherein
calcium signaling and/or beta-actin of the cells are correlated with changes in the brain electrical activity.

19. The method of claim 1, further comprising:
effecting an additional change, the additional change comprising at least one of:
regulating hypoxia and/or apoxia of the cells;
altering of expression of genes associated with Ras pathway, extracellular matrix, serine/threonine kinase, and/or cell junction;
altering cell signaling;
regulating PI3K/mTOR pathway in the cells; and
altering immune cells.

20. (canceled)

21. (canceled)

22. The method of claim 1, wherein
cell cycle arrest comprises arresting the cells in the G0/G1 and/or G2/M phases of the cell cycle.

23. The method of claim 19, wherein
altering immune cells comprises reducing B-cells and increasing T-cells.

24. The method of claim 19, wherein
altering cell signaling comprises alteration of microtubules and/or cytoskeletal markers.

25. (canceled)

26. The method of claim 1, wherein
altering cell membrane potential of the cells comprises reducing cell membrane potential of the cells and/or results in hyperpolarization of the cells.

27. (canceled)

28. (canceled)

29. (canceled)

30. The method of claim 1, wherein
changing a structural feature of the mitochondria comprises increasing size of the mitochondria and/or disorganizing cristae.

31. (canceled)

32. (canceled)

33. The method of claim 1, wherein
altering of expression of cytoskeletal genes comprises activating cytoskeleton related genes including Actin, Pectin, Spectrin, TRIOBP, kinases and/or a PDZ.

34. (canceled)

35. The method of claim 1, wherein
regulating FOXM1 in the cells comprises decreasing levels of FOXM1.

36. (canceled)

37. (canceled)

38. (canceled)

39. (canceled)

40. (canceled)

41. The method of claim 1, wherein
the cells include components of a tumor of an organism.

42. The method of claim 41, wherein
decreased growth or metabolism of the cells corresponds to decreased tumor size or burden.

43. (canceled)

44. The method of claim 41, further comprising
changing a tumor microenvironment (TME)
wherein changing a tumor microenvironment (TME) comprises reducing the percentage of B lymphocytes, increasing the percentage of cytotoxic T-cells, and/or reducing M2 tumor associated macrophages.

45. The method of claim 42, further comprising
decreasing nodules and/or mets in the organism.

46. (canceled)

47. A method of adjusting biological functioning of an entity, comprising:
inducing a change in a bioelectrical feature of a first portion of cells of the entity and/or mitochondria within the first portion of the cells, wherein the inducement effects:
a change in a structural aspect of a second portion of cells of the entity,
a change in a physiological aspect of the second portion of the cells; and
altering of expression of genes associated with the change in the bioelectrical feature, the change in the structural feature, and/or the change in the physiological feature.

48. (canceled)

49. (canceled)

50. (canceled)

51. The method of claim 47, wherein
the structural aspect comprises at least one of:
a structural feature of the mitochondria,
cytoskeleton organization of the cells,
membrane ion channels of the cells; and
the physiological aspect comprises at least one of:
an EMT marker in the cells,
energy metabolism in mitochondria of the cells, and
FOXM1 or FOXM1 regulatory proteins in the cells.

52. The method of claim 51, wherein
at least two of (a) the change in the bioelectrical feature, (b) the change in the structural aspect, (c) the change in the physiological aspect, and (d) the change in the expression of genes, correlates with at least one of:
cell cycle arrest of the cells and/or expression of cycle regulation proteins,
decreased cell-free DNA from the cells or intravasation,
decreased growth or metabolism of the cells,
deceased migration of the cells, and
decreased invasion of the cells.

53. The method of claim 52, wherein
inducing the change in the bioelectrical feature comprises administering a biofield therapy.

54. The method of claim 47, wherein
altering of expression of genes comprises at least one of an epigenetic effect, a change in transcriptomic signature, and a change in proteomic signature.

55. (canceled)

56. (canceled)

57. (canceled)

58. (canceled)

59. (canceled)

60. (canceled)

61. (canceled)

62. (canceled)

63. (canceled)

64. (canceled)

65. The method of claim 1, wherein
the cells include cells in an organoid, a patient-derived organoid, or a pancreatic cancer organoid.

66. (canceled)

67. The method of claim 1, wherein
the method decreases metastatic potential of the cells and/or cancerous cells.

68. The method of claim 67, wherein decrease of metastatic potential includes one or more of:
decreasing growth of the cells and/or cancerous cells;
decreasing size of a tumor comprising cancerous cells;
reducing the likelihood of occurrence of metastasis;
reducing migration or other metastatic behaviors of the cancerous cells or their progeny; and
reducing penetration or other metastatic capabilities of the cancerous cells or their progeny.

69. (canceled)

70. (canceled)

71. The method of claim 1, wherein
the cells include pancreatic ductal adenocarcinoma (PDAC) cells.

72. (canceled)
